(19) United States
(12) Patent Application Publication
(10) Pub. No.: US 2021/0222128 A1
(43) Pub. Date: Jul. 22, 2021

(54) INDUCIBLE TISSUE CONSTRUCTS AND USES THEREOF

(71) Applicants: Massachusetts Institute of Technology, Cambridge, MA (US); Trustees of Boston University, Boston, MA (US)

(72) Inventors: Christopher S. CHEN, Newton, MA (US); Sangeeta N. BHATIA, Lexington, MA (US); Arnav CHHABRA, Cambridge, MA (US); Amanda CHEN, East Cambridge, MA (US); Hyun Ho SONG, Long Island City, NY (US)

(21) Appl. No.: 16/953,002

(22) Filed: Nov. 19, 2020

Related U.S. Application Data
(60) Provisional application No. 62/964,477, filed on Jan. 22, 2020.

(51) Int. Cl. C12N 5/071; C12N 5/00; C12N 5/077; C12N 9/64; A61L 27/38

(52) U.S. Cl. CPC C12N 5/0671; C12N 5/0062; C12N 5/0656; C12N 9/6472; C12N 2502/1323; A61L 27/3895; A61L 27/3886; C12N 2513/00; C12Y 304/22062

(57) ABSTRACT

Inducible engineered tissue constructs comprising at least one cell population comprising a genetic construct are provided. Methods of making and using said constructs are also provided.

### CROSS-REFERENCE TO RELATED APPLICATIONS

This application claims the benefit of U.S. Provisional Patent Application Ser. No. 62/964,477, filed Jan. 22, 2020. The entire contents of which is incorporated herein by reference.

### STATEMENT REGARDING FEDERALLY SPONSORED RESEARCH OR DEVELOPMENT

This invention was made with Government support under Grant No. R01 EB008396 and R01 EB000262 awarded by the National Institutes of Health (NIH). The Government has certain rights in the invention.

### BACKGROUND OF THE DISCLOSURE

Three-dimensional (3D) tissue engineered models have evolved to encompass a range of applications spanning therapeutic cell-based therapies to in vitro organoid models. In all cases, recapitulation of physiologic functions and native tissue behavior is key to studying and harnessing complex, tissue-specific phenomena in normal and pathophysiological states. Compositions and methods for controlling various biological aspects within an engineered tissue, in vitro or in vivo, are needed.

### SUMMARY OF THE DISCLOSURE

In some aspects, the present disclosure provides an engineered tissue construct comprising one or more mammalian cell populations, wherein at least one cell population comprises a genetic construct comprising:

(i) a polynucleotide encoding a polypeptide of interest comprising an inducible element, wherein the polypeptide is activated upon interaction of the inducible element with a biological molecule or small molecule; or

(ii) a polynucleotide comprising an inducible promoter operably linked to a nucleotide sequence encoding a polypeptide or a nucleic acid molecule of interest, wherein expression of the polypeptide or nucleic acid molecule is controlled by the inducible promoter, wherein at least one cell population comprises parenchymal or non-parenchymal cells.

In any of the foregoing or related aspects, the polypeptide of interest of (i) or (ii) is a cell death-inducing polypeptide. In some aspects, the cell death-inducing polypeptide is an apoptosis-inducing polypeptide. In some aspects, the apoptosis-inducing polypeptide is selected from the group consisting of a caspase, thymidine kinase, cytosine deaminase, and p53 tumor suppressor. In some aspects, the caspase is an initiator caspase. In some aspects, the initiator caspase is selected from the group consisting of caspase 2, caspase 8, caspase 9 and caspase 10.

In any of the foregoing or related aspects, the polypeptide of interest of (i) is an initiator caspase operably linked to the inducible element. In some aspects, the polypeptide of interest of (i) is a caspase 9 monomer, and the inducible element is a dimerization domain. In some aspects, caspase 9 monomer is activated upon binding of a chemical inducer of dimerization (CID) to the dimerization domain.

In any of the foregoing or related aspects, the polypeptide of interest of (i) or (ii) induces cell proliferation in at least one cell population. In some aspects, cell proliferation comprises an increase in the number of cells and/or a change in proliferation markers in the cells. In some aspects, induction of cell proliferation results in overall expansion of the tissue construct. In some aspects, the polypeptide of interest is selected from the group consisting of Wnt2, epidermal growth factor (EGF), hepatocyte growth factor (HGF), fibroblast growth factor (FGF), vascular endothelial growth factor (VEGF), interleukin 8 (IL-8), angiotensin 2 (Ang-2), R-spondin-3 precursor (RSPO3), GATA-binding protein 4 (GATA4), interleukin 6 (IL-6), delta-like 4 (DLL4), inhibitor of DNA binding 1 (ID-1), prostaglandin E synthase 2 (PGE2) and colony stimulating factor 1 (CSF1).

In any of the foregoing or related aspects, the nucleic acid molecule of interest is an inhibitory nucleic acid molecule. In some aspects, the inhibitory nucleic acid molecule is an siRNA, an shRNA or an miRNA. In some aspects, the inhibitory nucleic acid molecule induces cell death. In some aspects, the inhibitory nucleic acid molecule induces apoptosis. In some aspects, the inhibitory nucleic acid molecule induces cell proliferation. In some aspects, the inhibitory nucleic acid molecule induces expansion of the engineered tissue construct. In some aspects, the inhibitory nucleic acid molecule induces tissue organogenesis. In some aspects, the inhibitory nucleic acid molecule induces differentiation of a stem cell or precursor cell in the engineered tissue construct.

In any of the foregoing or related aspects, the inducible promoter is activated by a small molecule. In some aspects, the small molecule is doxycycline, tetracycline or rapalog. In some aspects, the inducible promoter is a tetracycline-induced promoter. In some aspects, the inducible promoter is an isopropyl-beta-Isopropyl-beta-D-thiogalactopyranoside (IPTG)-regulated promoter.

In any of the foregoing or related aspects, the inducible promoter is activated by a biological molecule. In some aspects, the biological molecule is an RNA polymerase. In some aspects, the inducible promoter is a T7 RNA polymerase promoter or a T3 RNA polymerase promoter. In some aspects, the inducible promoter is a lactose-induced promoter or a steroid-regulated promoter.

In any of the foregoing or related aspects, the inducible promoter is activated by a thermal pulse, an ultrasound wave, an electric field, a light wave, or a magnetic field. In some aspects, the inducible promoter is a heat shock promoter.

In any of the foregoing or related aspects, the tissue construct comprises at least one population of parenchymal cells. In some aspects, the parenchymal cells are derived from liver, heart, kidney or pancreas. In some aspects, the parenchymal cells are hepatocytes or hepatocyte precursor cells. In some aspects, the parenchymal cells are stem cells or precursor cells capable of differentiating into a primary parenchymal cells.

In any of the foregoing or related aspects, the tissue construct comprises at least one population of non-parenchymal cells. In some aspects, non-parenchymal cells are stromal cells. In some aspects, the stromal cells are fibroblasts. In some aspects, the non-parenchymal cells are endothelial cells. In some aspects, the tissue construct comprises at least two populations of non-parenchymal cells. In some aspects, the at least two populations of non-parenchymal cells is a population of stromal cells and a population of endothelial cells.

In any of the foregoing or related aspects, the tissue construct comprises at least one population of parenchymal cells and at least one population of non-parenchymal cells. In some aspects, the parenchymal cells are hepatocytes or hepatocyte precursor cells, and the non-parenchymal cells are stromal cells. In some aspects, the tissue construct comprises two populations of non-parenchymal cells. In some aspects, the parenchymal cells are hepatocytes, and wherein the two populations of non-parenchymal cells is a population of stromal cells and a population of endothelial cells. In some aspects, the stromal cells are fibroblasts.

In other aspects, the disclosure provides an engineered tissue construct comprising:

(i) a population of hepatocytes; and

(ii) a population of stromal cells comprising a genetic construct comprising a polynucleotide encoding cell death-inducing polypeptide operably linked to an inducible element, wherein the polypeptide is activated upon interaction of the inducible element with a small molecule.

In any of the foregoing or related aspects, the cell death-inducing polypeptide is an initiator caspase, wherein the inducible element is a dimerization domain, and wherein the small molecule is a CID. In some aspects, the cell death-inducing polypeptide is an initiator caspase. In some aspects, the initiator caspase is caspase 9.

In any of the foregoing or related aspects, the population of stromal cells comprises fibroblasts.

In any of the foregoing or related aspects, the engineered tissue construct further comprises a population of endothelial cells. In some aspects, the population of endothelial cells is seeded within at least one pre-templated vessel within a substrate.

In any of the foregoing or related aspects, the engineered tissue construct further comprises a biocompatible scaffold, wherein the one or more cell populations is cultured in the scaffold. In some aspects, the substrate or biocompatible scaffold is a hydrogel scaffold.

In any of the foregoing or related aspects, the engineered tissue construct is two-dimensional. In other aspects, the engineered tissue construct is three-dimensional.

In any of the foregoing or related aspects, the one or more mammalian cell populations are human cells.

In other aspects, the disclosure provides a method for eliminating a population of cells within an engineered tissue construct, comprising:

(a) introducing a genetic construct into a first cell population, wherein the genetic construct comprises a polynucleotide encoding a cell death-inducing polypeptide operably linked to an inducible element, wherein the polypeptide is activated upon interaction of the inducible element with a biological molecule or small molecule;

(b) co-culturing the cells of (a) with a second cell population on a substrate to form the engineered tissue construct; and

(c) contacting the tissue construct with the biological molecule or small molecule to activate the cell death-inducing polypeptide, such that the first cell population is eliminated from the tissue construct.

In yet other aspects, the disclosure provides a method for eliminating a population of cells within an engineered tissue construct, comprising:

(a) introducing a genetic construct into a first cell population, wherein the genetic construct comprises a polynucleotide comprising an inducible promoter operably linked to a nucleotide sequence encoding a cell death-inducing polypeptide or nucleic acid molecule, wherein expression of the polypeptide or nucleic acid molecule is controlled by the inducible promoter;

(b) co-culturing the cells of (a) with a second cell population on a substrate to form the engineered tissue construct;

(c) contacting the tissue construct with a stimulus of the inducible promoter to express the cell death-inducing polypeptide or nucleic acid molecule, such that the first cell population is eliminated from the tissue construct.

In any of the foregoing or related aspects, the cell death-inducing polypeptide is an initiator caspase, wherein the inducible element is a dimerization domain, and wherein the caspase is activated with a CID. In some aspects, the cell death-inducing polypeptide is an initiator caspase. In some aspects, the initiator caspase is caspase 9.

In any of the foregoing or related aspects, step (b) further comprises co-culturing the cells with a third cell population comprising endothelial cells. In some aspects, the endothelial cells self-assemble into vasculature. In other aspects, the third cell population is seeded into pre-templated vessels in the substrate to promote vascularization of the engineered tissue construct. In some aspects, eliminating the cells of (a) does not disrupt self-assembly of vascularization.

In further aspects, the disclosure provides a method for inducing expansion of an engineered tissue construct, comprising:

(a) introducing a genetic construct into at least one population of cells, wherein the genetic construct comprises a polynucleotide encoding a polypeptide of interest comprising an inducible element, wherein the polypeptide is activated upon interaction of the inducible element with a biological molecule or small molecule;

(b) culturing the cell population of (a), with or without another cell population, onto a substrate to form a tissue construct; and

(c) contacting the tissue construct with the biological molecule or small molecule, such that expression of the polypeptide induces expansion of the engineered tissue construct.

In yet further aspects, the disclosure provides a method for inducing expansion of an engineered tissue construct, comprising:

(a) introducing a genetic construct into at least one population of cells, wherein the genetic construct comprises a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;

(b) culturing the cell population of (a) onto a substrate, with or without another cell population, to form a tissue construct; and

(c) contacting the tissue construct with a stimulus of the inducible promoter, such that expression of the polypeptide induces expansion of the engineered tissue construct.

In any of the foregoing or related aspects, the one or more cell populations comprise a population of endothelial cells. In some aspects, the endothelial cells self-assemble into vasculature. In other aspects, the endothelial cells are cultured into pre-templated vessels in the substrate.

In any of the foregoing or related aspects, one or more cell populations comprise a population of hepatocytes or hepatocyte precursor cells. In any of the foregoing or related aspects, the one or more cell populations comprise a population of stromal cells. In some aspects, the stromal cells are fibroblasts.

In any of the foregoing or related aspects, the polypeptide of nucleic acid molecule of interest induces cell proliferation in at least one cell population. In some aspects, cell proliferation comprises an increase in the number of cells and/or a change in proliferation markers in the cells.

In any of the foregoing or related aspects, the polypeptide of interest is selected from the group consisting of Wnt2, epidermal growth factor (EGF), hepatocyte growth factor (HGF), fibroblast growth factor (FGF), vascular endothelial growth factor (VEGF), interleukin 8 (IL-8), angiotensin 2 (Ang-2), r-spondin-3 precursor (RSPO3), GATA-binding protein 4 (GATA4), interleukin 6 (IL-6), delta-like 4 (DLL4), inhibitor of DNA binding 1 (ID-1), prostaglandin E synthase 2 (PGE2) and colony stimulating factor 1 (CSF1).

In any of the foregoing or related aspects, step (c) occurs in vivo. In other aspects, step (c) occurs in vitro.

In any of the foregoing or related aspects, the cells are human cells.

In some aspects, the disclosure provides a kit comprising an engineered tissue construct comprising one or more mammalian cell populations, wherein at least one cell population comprises a genetic construct comprising a polynucleotide encoding a cell death-inducing polypeptide operably linked to an inducible element, wherein the polypeptide is activated upon interaction of the inducible element with a biological molecule or small molecule, and instructions for contacting the tissue construct with the biological molecule or small molecule to activate the cell death-inducing polypeptide.

In further aspects, the disclosure provides a kit comprising an engineered tissue construct comprising one or more mammalian cell populations, wherein at least one cell population comprises a genetic construct comprising a polynucleotide comprising an inducible promoter operably linked to a nucleotide sequence encoding a polypeptide or nucleic acid molecule of interest, wherein expression of the polypeptide or nucleic acid molecule is controlled by the inducible promoter, and instructions for contacting the tissue construct with a stimulus of the inducible promoter to induce expansion or tissue organogenesis of the engineered tissue construct.

In yet further aspects, the disclosure provides a method for treating a metabolic disorder in a subject in need thereof, the method comprising implanting an engineered tissue construct described herein into the subject. In some aspects, the metabolic disorder is selected group the group consisting of: Citrullinemia type I, Ornithine transcarbamylase deficiency, Carbamoyl phosphate synthetase 1 deficiency, Arginase deficiency, Factor VII deficiency, Hemophilia A, Hemophilia B, Factor X deficiency, Familial hypercholesterolemia, Crigler-Najjar syndrome, Phenylketonuria, Primary hyperoxaluria type I, Argininosuccinic aciduria, Alpha-1 antitrypsin deficiency, Hereditary hemochromatosis, Glycogen storage disease type I, Hereditary tyrosinemia, acute liver failure, acute-on-chronic liver disease.

In any of the foregoing aspects, the engineered tissue construct recapitulates at least one function of an organ. In some aspects, the organ is a liver and at least one function is albumin secretion. In some aspects, the organ is liver and the function is albumin secretion and urea secretion.

In other aspects, the disclosure provides a method of treating chronic liver failure in a subject, comprising implanting an engineered tissue construct described herein into the subject.

In any of the foregoing or related aspects, a method herein comprises administering to the subject a stimulus of the inducible element or inducible promoter prior to implanting the engineered tissue construct. In other aspects, the method comprises administering to the subject a stimulus of the inducible element or inducible promoter after implanting the engineered tissue construct.

In some aspects, the disclosure provides an engineered tissue construct described herein for use in treating a metabolic disorder in a subject in thereof, wherein the engineered tissue construct is implanted into the subject.

In other aspects, the disclosure provides an engineered tissue construct described herein for use in treating chronic liver failure in a subject in need thereof, wherein the engineered tissue construct is implanted into the subject.

### BRIEF DESCRIPTION OF THE FIGURES

FIG. 1A is a schematic showing J2 fibroblasts bearing an inducible caspase 9 (iCasp9)-green fluorescent protein (GFP) gene. A chemical inducer of dimerization (CID) is used to induce iCasp9 dimerization, leading to apoptosis and elimination of the cells from culture.

FIG. 1B provides graphs showing the intensity of GFP expression and % GFP expressing cells in stable iCasp9-GFP-J2s in passages 16-22 of cell culture.

FIG. 1C provides images of CID-induced dimerization of iCasp9 unimers in untreated, vehicle treated, or 50 nM CID treated iCasp9-GFP J2s and collected 15 and 30 minutes after treatment. Cells were stained with Hoechst (nuclear), GFP (J2s), and Caspase-9 (apoptosis). After CID treatment, Caspase-9 staining increased. (scale bar=50 μm).

FIG. 1D is a bar graph showing CID induced activation of caspase-9 cleavage activity in iCasp9-GFP-J2s treated with vehicle or CID. Vehicle treated cells were processed for imaging 60 minutes following treatment and CID treated cells were processed 15 or 30 minutes following treatment (****p<0.0001 vs. time-matched, dose matched J2s, n−3).

FIG. 1E is a graph showing quantification of flow cytometry analysis of CID-treated iCasp9-GFP J2s stained with Annexin V and SYTOX to quantify extent of apoptotic activity (100,000 events were measured). Control J2 cells were treated with vehicle or CID for 60 minutes and iCasp9-GFP-J2s were treated with vehicle, or CID for 15, 30, or 60 minutes.

FIG. 1F provides images showing cell death in CID-treated iCasp9-GFP J2s. Cells were treated with vehicle or 50 nM CID for 24 hours. Cells were imaged using GFP and Hoechst for nuclear staining.

FIG. 1G is a graph showing cell viability after treatment with low doses (500 nM, 50 nM, and 5 nM) of CID. Cells were collected for imaging 60 minutes after exposure.

FIG. 2A is a graph showing albumin secretion in micro-patterned co-cultures (MPCCs; hepatocytes cultured with J2s) or pure hepatocytes (“no J2”) (n=3). MPCCs consisted of ˜10,000 hepatocytes and ˜7,000 fibroblasts after seeding. Supernatant was collected on different days and measured for albumin secretion.

FIGS. 2B-2D are graphs showing albumin secretion (FIG. 2B), urea secretion (FIG. 2C), and basal expression of cytochrome P450 3A4 (CYP3A4) (FIG. 2D) in MPCCs containing wild-type J2s or iCasp9-GFP-J2s. Supernatant was collected on different days and used for analysis.

FIG. 2E is a schematic demonstrating removal of iCasp9-GFP J2s by apoptosis following CID treatment in MPCCs.

FIG. 2F provides phase contrast images of vehicle or CID-treated MPCCs (scale bar=250 μM). Arrows indicate apoptotic bodies, dotted lines demarcate hepatocyte islands. Cells were treated with 50 nM CID for 2 hours.

FIG. 2G is a graph showing albumin secretion for MPCCs treated with vehicle, 50 nM CID, or supernatant from apoptotic cells. Supernatant was collected for measurement at different days.

FIGS. 2H and 2I are graphs depicting albumin secretion after co-culture of MPCCs treated with 50 nM CID at day 1, 3, or 7 (FIG. 2H) and experimental repeat showing reproducibility of fibroblast dependence in MPCC from FIG. 2H (FIG. 2I). Secretion level was normalized to day 13, arrows indicate dose day.

FIG. 3A shows images and quantification of compaction by circularity from hepatocytes and fibroblasts cultured in microwells at increasing fibroblast:hepatocyte ratios for 24-hours (n=5, *p<0.05) vs. stromal cell-matched 1:1 spheroids. J2 fibroblasts or iCasp9-J2 fibroblasts were used.

FIG. 3B is a graph showing albumin secretion for 3D cultures consisting of pure hepatocytes (“no J2”) or hepatocytes and fibroblasts (“co-culture spheroid”) measured from supernatant over time (n=9). Cells were seeded at a 1:4 (hepatocyte:fibroblast) ratio.

FIGS. 3C-3E provide graphs measuring albumin secretion (FIG. 3C), urea secretion (FIG. 3D) and CYP3A4 activity on day 10 (FIG. 3E) in J2s and iCasp9-GFP J2s co-cultured with hepatocytes in spheroid-laden hydrogels. Supernatant was collected for analysis over a 10-day period. Cells were seeded at a 1:4 (hepatocyte:fibroblast) ratio.

FIG. 3F is a graph showing cell viability for monoculture iCasp9-GFP fibroblasts encapsulated in hydrogel then treated with CID at varying doses and assayed for viability (n=3). Cells were seeded at a 1:4 (hepatocyte:fibroblast) ratio.

FIG. 3G is a graph measuring fibroblast re-growth in hepatocyte/fibroblast co-culture after 21 days following treatment on day 1, 3, or 7 with 50 nM CID or vehicle. n.d.=not detected. Cells were seeded at a 1:4 (hepatocyte:fibroblast) ratio.

FIG. 3H is a schematic depicting culture of hepatocytes with fibroblasts in microwell molds and treated with CID to removed fibroblasts via apoptosis.

FIG. 3I provides immunofluorescent images showing fibroblast elimination in spheroid-laden hydrogels (3D culture of hepatocytes and iCasp9-GFP J2s) seeded at a 1:4 (hepatocyte:fibroblast) ratio. Spheroids were treated with vehicle or 50 nM CID at day 1 and imaged at 1, 2, 3, 5, and 7-days. Hepatocytes are marked using CellTracker (dark grey) and iCasp9-GFP J2s are shown in light grey. The CID treated cultures show negligible iCasp9-GFP staining starting at day 2 (scale bar=100 μm).

FIG. 3J is a graph measuring albumin secretion from supernatant for 3-weeks in spheroid-laden hydrogels seeded at a 1:4 (hepatocyte:fibroblast) ratio. Cells were dosed with 50 nM CID on day 1 after co-culture initiation.

FIG. 3K shows a treatment schematic (left) and following quantification (right) of fibroblast-depleted (CID) and fibroblast-intact (vehicle) co-culture with human primary hepatocytes (PHH) and treated with rifampin (2504) for 72 hours (starting on day 5). Cells were then assayed for induction of CYP3A4 (CYP).

FIG. 3L provides graphs showing albumin secretion in spheroid-laden hydrogels treated with 50 nM CID at day 1 (left), 3 (middle), or 7 (right) after initiating co-culture (n=9, normalized to day 15, arrows indicate dose day).

FIG. 4A is a schematic depicting the process of seeding a microfluidic device with a fibrin gel containing human umbilical vein cells (HUVECs) and human dermal fibroblasts (HDFs) cast around two needle-molded endothelized microfluidic channels (i) and a close-up illustration of the formed tissue inside the microfluidic device (ii).

FIG. 4B provides representative max projections of devices fixed on day 7 with varying HUVEC:HDF ratios. Endothelial networks were perfused with 500 kDa dextran (light grey) through one parent channel to visualize connected luminal networks. HUVECS were labeled with Ulex Europaeus Agglutinin (UEA; dark grey).

FIGS. 4C-4E are graphs showing quantification of percentage of branching nodes (FIG. 4C), percentage of vessels perfused (FIG. 4D), and average diameter of perfused vessels (i) and the relative frequency of each diameter (ii) (FIG. 4E) in devices seeded at different HUVEC:HDF ratios and fixed on day 7. # represents where data points removed due to no vessels being perfused in those devices. Each point represents one device. *: p<0.05, ****: p<0.0001, ns=not significant.

FIG. 4F provides images showing multiphoton max projection of a representative 3:6 ratio (HUVEC:HDF embedded in fibrin) device collected at day 7, demonstrating vascularization throughout the thickness of the engineered tissue. (scale bars=100 μm).

FIG. 5A provides representative images of growth progression from multicellular structures to functionally connected vasculature using HUVECs. GFP expressing HUVECS were seeded in the needle lumen only and eventually complex with the endothelial cells originating in the bulk gel (i). 2.5× zoomed images of the center of the devices (ii). Same devices shown in (i) with dextran perfusion shown along with UEA lectin (iii). Scale bars (i) and (iii)=150 μm, (ii)=50 μm.

FIGS. 5B-5D are graphs showing quantification of percentage of branching nodes (FIG. 5B), percentage of vessels perfused (FIG. 5C), and average diameter of perfused vessels in devices (FIG. 5D) seeded at a 3:6 ratio of HUVEC:HDF and fixed at day 1, 3, 5, or 7. # represents where data points removed due to no vessels being perfused in those devices. *: p<0.05, ****: p<0.0001, ns=not significant.

FIG. 6A is a graph demonstrating that iCasp9-HDFs initiate apoptosis upon the addition of CID. Different concentrations of CID (5, 50, and 500 nM) were added to a 2D culture of iCasp9-HDFs, and the ATP level was measured at different timepoints for 6 hours.

FIGS. 6B-6C are images and quantification of iCasp9-HDFs plated on 2D wells, treated with a vehicle control or 10 nM CID for 24 hours, fixed, stained with phalloidin and DAPI, and imaged (FIG. 6B); quantification of cell density of vehicle control and CID treated cells (FIG. 6C)

FIG. 6D provides time-lapse images of both device conditions (vehicle or CID treated for 0-18 hours) in the microfluidic device. Cells were live-imaged (dark grey=mRuby-LifeAct-HUVEC, white=iCasp9-GFP HDF) stained with [stain]. Scale bars=100 μm.

FIG. 7A is a schematic of the experimental timeline from seeding in the device to fixation. CID was administered at day 0, 1, 3, or 5 (as indicated by the light grey bars).

FIG. 7B provides representative images of max projections of devices treated with 10 nM CID or vehicle control on a designated day and fixed on day 7. Devices were stained with Ulex Europaeus Agglutinin (UEA) and perfused with dextran.

FIG. 7C provides graphs showing the quantification of percentage of branching nodes (i), percentage of perfused vessel segments (ii), and average vessel diameter of perfused vessels (iii) from cells treated with 10 nM CID or vehicle control on a designated day and fixed on day 7 # represents where data points removed due to no vessels being perfused in those devices. ***: p<0.001, ****: p<0.0001, ns=not significant.

FIG. 7D provides images showing transient support of human lung fibroblasts (HLFs) is sufficient to drive functional vascular morphogenesis. CID was added to the HUVEC/iCasp9-HLF co-culture devices on day 1 or day 3, and all devices were fixed at day 7. Top row shows UEA-stained HUVECs (white) at day 7. Bottom row shows merged images of UEA-stained HUVECs (dark grey) and FITC-conjugated dextran (light grey, along with low GFP (white) signal from iCasp9-HLFs). Scale Bars=150 μm.

FIG. 8A is a schematic showing the experimental timeline for devices fixed after CID treatment. Tri-culture (HUVEC, iCasp9-HDFs, and primary human hepatocytes) devices were used to analyze the function of vascularized engineered hepatic tissues.

FIG. 8B provides images of representative max projections of dextran-perfused vehicle control- and 10 nM CID treated tri-culture devices stained for UEA and human arginase 1 (i) and 2.5× images (ii). Scale bars (i)=150 μm and (ii) 50 μm.

FIG. 8C provides graphs showing quantification of secreted human albumin (i) and urea production (ii) in each tri-culture device with mean line shown. Dotted line denotes day vehicle control and 10 nM CID were dosed for 24 hours. *: p<0.05, **: p<0.01

FIG. 9A provides images showing removal of iCasp9-HLF (light grey) from HUVEC (dark grey)/iCasp9-HLF tissue constructs implanted in mouse fat pads. Mice were injected intraperitoneally with 10 mg/kg CID or vehicle on days 5, 7, and 9 post-implantation. Scale bar=100 μm.

FIG. 9B provides images showing formed vessel structures in HUVEC/iCasp9-HLF tissue constructs implanted in mouse fat pads. Mice were injected with CID or vehicle on days 5, 7, and 9. Implants were collected on day 10 post-implantation and stained for red blood cells (anti-TER119; light grey). Scale bar=50 μm.

FIG. 9C provides a graph showing quantification of secreted human albumin in the plasma of mice transplanted with hepatocyte/iCasp9-HDF/HUVEC tissue constructs. Constructs were cultured for 7 days then transplanted into mouse fat pads. 10 mg/kg CID or vehicle was administered intraperitoneally on days 5 and 6 post-implantation and blood was collected on day 7 post-implantation. Dots represent individual animals. n.s. as determined by 1-way ANOVA testing with Tukey's multiple comparison test.

### DETAILED DESCRIPTION

The present disclosure provides engineered tissue constructs in which one or more mammalian cell populations comprises an inducible genetic construct to allow control of tissue organogenesis and/or tissue expansion. The disclosure is based, at least in part, on the discovery that tissue organogenesis and/or expansion of engineered tissue constructs can be controlled by engineering at least one cell population within the tissue construct with an inducible biological element, such as an inducible polypeptide or an inducible nucleic acid (e.g., inducible promoter).

Engineered tissue constructs, such as liver tissue constructs, require heterotypic and homotypic cell-cell interactions. For example, stromal cells (e.g., fibroblasts) have been shown to support the formation and stabilization of tissue constructs in vitro, but such cells may be less desirable for clinical translation of the tissue construct in vivo. It has now been shown that supporting cells, such as stromal cells, engineered to comprise an inactive, constitutively expressed cell death-inducing polypeptide can be eliminated from a tissue construct comprising parenchymal cells upon contact with a small molecule that activates the cell death-inducing polypeptide. Unexpectedly, the parenchymal cells (e.g., hepatocytes) continue to function after elimination of the supporting cell population. Without being bound by theory, the engineered tissue constructs described herein can be used to investigate the temporal role of support cells in tissue organogenesis and/or function of a tissue construct.

As described in the Examples, fibroblasts engineered to express inducible caspase 9, i.e., caspase 9 unimers that become activated by chemically induced dimerization (CID), were co-cultured with hepatocytes to form a liver tissue construct. Upon dimerization of caspase 9 unimers, in vitro or in vivo after implantation of the construct, apoptosis was activated and fibroblasts were eliminated from the tissue construct while function of the hepatocytes was maintained. Notably, hepatocyte function was only maintained in three-dimensional tissue constructs. Elimination of fibroblasts from two-dimensional tissue constructs was detrimental to hepatocyte function. As demonstrated herein, fibroblasts are necessary to form the liver tissue construct in vitro for both two-dimensional and three-dimensional constructs, but are not required to maintain hepatic functions (e.g., albumin secretion) of three-dimensional tissue construct. It is also shown herein there may be a window of opportunity for eliminating a cell population to avoid a negative impact on the overall function of the tissue construct. For example, when fibroblasts were eliminated from a three-dimensional liver tissue construct 3 or 7 days after hepatocyte-fibroblast co-culture, hepatocyte function was negatively impacted. Without being bound by theory, the timing for eliminating a cell population from a tissue construct is dependent on various factors, such as the type of cells and type of scaffold being utilized. Accordingly, the present disclosure provides compositions and methods for eliminating a cell population that may not be required for maintaining the function of a tissue construct after a certain period of time.

It is believed the engineered tissue constructs recapitulate the native microenvironment of a tissue, which prolongs the longevity and function of cells within the tissue construct. Specifically, the engineered tissue constructs described herein provide recapitulating cues from the native tissue microenvironment, which can be temporally controlled with by an inducible genetic construct described herein.

The disclosure also provides engineered vascularized tissue constructs. It has been further demonstrated that supporting cells, such as stromal cells, enhance self-assembly of vasculature throughout a tissue construct. For example, self-assembly of vessels from endothelial cells was found to depend on the presence of fibroblasts. However, as demonstrated herein, elimination of supporting cells after formation of vasculature does not have a negative impact on the vascular structure. It is believed support cells are needed during the initial stage of vasculogenesis of endothelial cells but that fibroblasts can be removed shortly thereafter. This finding was further supported based on the observation that elimination of stromal cells did not negatively impact the vascularization of tissue constructs having organ-specific parenchymal cells nor did it negatively affect the parenchymal cells. For example, elimination of fibroblasts did not negatively affect the secretive functions of hepatocytes in vascularized tissue constructs. Surprisingly, elimination of fibroblasts increased secreted albumin and urea levels by hepatocytes compared to tissue constructs retaining the fibroblasts.

Also demonstrated herein is the surprising discovery that implanted tissue constructs demonstrated the ability to successfully integrate with host vessels, whether cells are eliminated from a tissue construct pre- or post-implantation of the construct.

The disclosure also provides methods and compositions for controlling angiogenesis of an engineered tissue construct, by for example, introducing a genetic construct comprising a polynucleotide comprising an inducible promoter operably linked to a nucleotide sequence encoding an angiogenic factor.

Based, at least in part, on the demonstration of controlling apoptosis within a tissue construct, various aspects of tissue organogenesis and tissue expansion can be similarly controlled as described herein.

Accordingly, in some aspects, the present disclosure provides engineered tissue constructs comprising at least one cell population engineered to comprise an inducible biological element (e.g., an inducible polypeptide or an inducible nucleic acid).

### Engineered Tissue Constructs

In some aspects, the disclosure provides an engineered tissue construct comprising one or more mammalian cell populations, wherein at least one cell population comprises a genetic construct comprising:

(i) a polynucleotide encoding a polypeptide of interest comprising an inducible element, wherein the polypeptide is activated upon interaction of the inducible element with a stimulus (e.g., a biological molecule or a small molecule); or

(ii) a polynucleotide comprising an inducible promoter operably linked to a nucleotide sequence encoding a polypeptide or nucleic acid molecule of interest, wherein expression of the polypeptide or nucleic acid molecule is controlled by the inducible promoter,

wherein at least one cell population comprises parenchymal or non-parenchymal cells.

### Genetic Constructs

In some embodiments, an engineered tissue construct described herein comprises at least one cell population engineered to comprise a genetic construct described herein. In some embodiments, the genetic construct is a polynucleotide encoding a polypeptide of interest comprising an inducible element, wherein the polypeptide is activated upon interaction of the inducible element with a stimulus (e.g., a biological molecule or small molecule). In some embodiments, the genetic construct is a polynucleotide comprising an inducible promoter operably linked to a nucleic acid molecule encoding a polypeptide of interest, wherein expression of the polypeptide is controlled by the inducible promoter.

In some embodiments, a population of non-parenchymal cells is engineered to comprise a genetic construct. In some embodiments, a population of parenchymal cells is engineered to comprise a genetic construct. In some embodiments, a population of parenchymal cells is engineered to comprise a first genetic construct and a population of non-parenchymal cells is engineered to comprise a second genetic construct, wherein the first and second genetic constructs are different.

In some embodiments, an engineered tissue construct described herein comprises at least two cell populations, wherein at least one of the cell populations is comprised of parenchymal cells, and at least one of the cell populations is engineered to comprise a genetic construct.

In some embodiments, an engineered tissue construct described herein comprises at least two cell populations, wherein at least one of the cell populations is comprised of parenchymal cells, and at least one of the cell populations is comprised of non-parenchymal cells engineered to comprise a genetic construct.

In some embodiments, an engineered tissue construct described herein comprises at least three cell populations, wherein at least one of the cell populations is comprised of parenchymal cells, and wherein at least two of the cell populations are engineered to comprise a genetic construct. In some embodiments, the two engineered cell populations comprise different genetic constructs.

1. Polypeptides with Inducible Element

In some embodiments, the genetic construct is a polynucleotide encoding a polypeptide of interest comprising an inducible element. In some embodiments, the polypeptide of interest is a fusion protein comprising the inducible element.

In some embodiments, the polypeptide of interest is linked to the inducible element without a linker. In some embodiments, the polypeptide of interest is linked to the inducible element with a linker.

In some embodiments, the inducible element is a ligand-binding domain. The ligand-binding domain can be any convenient domain that will allow for induction using a natural or unnatural ligand, for example, an unnatural synthetic ligand. In some embodiments, the ligand-binding domain is a multimerization domain. The multimerizing domain or ligand-binding domain can be internal or external to the cellular membrane, depending upon the nature of the construct and the choice of ligand. A wide variety of ligand-binding proteins, including receptors, are known.

As used herein the terms “ligand-binding domain” and “ligand-binding region” can be interchangeable with the term “receptor”. Of particular interest are ligand-binding proteins for which ligands (for example, small organic ligands) are known or may be readily produced. These ligand-binding domains or receptors include the FKBPs and cyclophilin receptors, the steroid receptors, the tetracycline receptor, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In some embodiments, the ligand-binding region is selected from the group consisting of FKBP ligand-binding region, cyclophilin receptor ligand-binding region, steroid receptor ligand-binding region, cyclophilin receptors ligand-binding region, and tetracycline receptor ligand-binding region. In some embodiments, the ligand binding region is an FKBP ligand-binding region.

In some embodiments, the ligand-binding domains or receptor domains will be at least about 50 amino acids, and fewer than about 350 amino acids, usually fewer than 200 amino acids, either as the natural domain or truncated active portion thereof. The binding domain may, for example, be small (<25 kDa, to allow efficient transfection in viral vectors), monomeric, nonimmunogenic, have synthetically accessible, cell permeable, nontoxic ligands that can be configured for dimerization.

The ligand-binding domain can be intracellular or extracellular depending upon the design of the construct and the availability of an appropriate ligand. For hydrophobic ligands, the binding domain can be on either side of the membrane, but for hydrophilic ligands, particularly protein ligands, the binding domain will usually be external to the cell membrane, unless there is a transport system for internalizing the ligand in a form in which it is available for binding. For an intracellular receptor, the construct can encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

In some embodiments, antibodies and antibody subunits, e.g., heavy or light chain, particularly fragments, more particularly all or part of the variable region, or fusions of heavy and light chain to create high-affinity binding, are used as the binding domain.

In some embodiments, the ligand is a biological molecule or a small molecule. In some embodiments, binding of the ligand binding domain activates the polypeptide of interest.

In some embodiments, the inducible element is a multimerization domain. In some embodiments, the multimerization domain is an FKBP12 region. In some embodiments, the ligand of the multimerization domain is an FK506 dimer or a dimeric FK506 analog ligand. In some embodiments, the ligand is AP1903, AP20187, or any derivative or analog known in the art that promotes multimerization. In some embodiments, multimerization activates the polypeptide of interest.

In some embodiments, the ligand of the multimerization domain is a chemical inducer of dimerization (CID). In some embodiments, the CID is rapamycin or a rapamycin analog (“rapalogs”) which have improved or differing pharmacodynamic or pharmacokinetic properties to rapamycin but have the same broad mechanism of action. In some embodiments, the rapalog is selected from, but not limited to, Sirolimus, Everolimus, Temsirolimus, and Deforolimus. In some embodiments, the rapalog is selected from, but not limited to, C-20-methyllyrlrapamycin (MaRap); C16(S)-Butylsulfonamidorapamycin (C16-BS-Rap); C16-(S)-3-mehylindolerapamycin (C16-iRap); and C16-(S)-7-methylindolerapamycin (AP21976/C16-AiRap). In some embodiments, the chemical inducer of dimerization is photo-switchable as described in EP. Pat. No. EP3424922, herein incorporated by reference.

### 2. Inducible Promoter Controlling Expression

In some embodiments, the genetic construct is a polynucleotide comprising an inducible promoter operably linked to a nucleotide sequence encoding a biological molecule of interest (e.g., a nucleic acid molecule or a polypeptide), wherein expression of the biological molecule is controlled by the inducible promoter.

In some embodiments, an inducible promoter comprises a transcription modulator responsive element, and thus expression is controlled by a transcription modulator.

In some embodiments, the inducible promoter is based on a prokaryotic operon, e.g., the lac operon, transposon Tn10, tetracycline operon, and the like. In some embodiments, the inducible promoter is based on a eukaryotic signaling pathway, e.g. steroid receptor-based expression systems, e.g. the estrogen receptor or progesterone-based expression system, the metallothionein-based expression system; the ecdysone-based expression system. As such, transcription modulators and transcription modulator responsive elements may be derived from a variety of different wild-type systems.

In some embodiments, the genetic construct comprising an inducible promoter is chromosomally integrated or episomally maintained in the cell or population of cells, as desired. When chromosomally integrated, the genetic construct in stably part of a chromosome of the host cell. When episomally maintained, the genetic construct is present on a vector, e.g., a plasmid, an artificial chromosome, e.g. BAC, that is not part of a host cell's chromosome.

In some embodiments, an inducible promoter comprises both a transcription modulator responsive element and a minimal promoter element which are operably linked to each other, where the transcription modulator responsive element may be either upstream or downstream from the minimal promoter element, depending on the particular configuration of the genetic construct. In some embodiments, transcription modulator responsive elements are prokaryotic operon operator sequences, e.g., tet operator sequences. In some embodiments, a transcription modulator responsive element includes multiple copies (e.g., multimerized or concatemerized copies) of 2 or more operator sequences. Minimal promoter sequences are sequences which are not themselves transcribed but which serve (at least in part) to position the transcriptional machinery for transcription. The term “minimal promoter” includes partial promoter sequences which define the start site of transcription for a linked coding sequence to be transcribed but which by themselves are not capable of initiating transcription efficiently, if at all. Thus, the activity of such a minimal promoter is dependent upon the binding of a transcription modulator (e.g., a transcription modulator protein) to an operatively linked transcription modulator responsive element. Minimal promoters of interest include, but are not limited to: the minimal promoter from the human cytomegalovirus (e.g., nucleotide positions between +75 to −53, nucleotide positions between +75 to −31, etc.); the human HSV thymidine kinase promoter; the human U6 promoter and the like. Promoters of interest include those described, e.g., in Gossen, M. and Bujard, H. (1992) Proc. Natl. Acad. Sci. USA 89:5547-5551. While the length of the minimal promoter element may vary, in some instances the length of this element ranges from 25 to 1000, such as 50 to 100 base pairs.

In some embodiments, a transcription modulator responsive element and a minimal promoter are separated by a linker sequence, which may be any convenient sequence. In such embodiments, the distance between the transcription modulator responsive element and the minimal promoter may vary.

In some embodiments, the inducible promoter comprising a transcription modulator responsive promoter element is operably linked to a coding sequence (e.g., a nucleotide sequence encoding a polypeptide or a nucleic acid molecule), such that upon binding of the transcription modulator to the transcription modulator responsive element, the coding sequence is expressed. In some embodiments, the coding sequence of the genetic construct encodes a polypeptide of interest or a nucleic acid of interest (e.g., an inhibitory RNA). Thus, upon induction of transcription of the coding sequence of the genetic construct and translation of the resultant mRNA, in some embodiments the polypeptide of interest is produced. Alternatively, in some embodiments, the coding sequence to be transcribed encodes for an active RNA molecule, e.g., an antisense RNA, sRNA, ribozyme, miRNA, etc. In some embodiments, expression of active RNA molecules are used to regulate functions within the host (e.g., prevent the production of a protein of interest by inhibiting translation of the mRNA encoding the protein). While the length of the coding sequence may vary, in some embodiments the coding sequence has a length ranging from 10 bp to 15,000 bp, such as 50 bp to 5,000 bp and including 100 bp to 1000 bp.

In some embodiments, the coding sequence of the genetic construct is exogenous or endogenous. An “exogenous” coding sequence is a nucleotide sequence which is introduced into the host cell, e.g., into the genome of the host. The exogenous coding sequence may not be present elsewhere in the genome of the host (e.g., a foreign nucleotide sequence) or may be an additional copy of a sequence which is present within the genome of the host but which is integrated at a different site in the genome. An “endogenous” coding sequence is a nucleotide sequence which is present within the genome of the host. An endogenous gene can be operatively linked to an inducible promoter by homologous recombination between an inducible promoter sequence recombination vector and sequences of the endogenous gene, such that the native promoter is replaced with the regulatory protein responsive element and the endogenous gene becomes part of an inducible expression cassette.

In some embodiments, the nucleic acid molecule encoded by a polynucleotide described herein is an antisense oligonucleotide. Antisense oligonucleotides are capable of blocking or decreasing the expression of a desired target gene by targeting nucleic acids encoding the gene or subunit thereof. Methods are known to those of ordinary skill in the art for the preparation of antisense oligonucleotide molecules that will specifically bind one or more target gene(s) without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiments, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugarmodifications known to those of ordinary skill in the art.

In some embodiments, the nucleic acid molecule encoded by a polynucleotide described herein is an inhibitory RNA molecule. RNA interference (RNAi) is a biological process in which RNA molecules inhibit gene expression or translation by neutralizing targeted mRNA molecules. Specifically, RNAi refers to a post-transcriptional silencing mechanism initiated by small double-stranded RNA molecules that suppress expression of genes with sequence homology. Key to the mechanism of RNAi are small interfering RNA (siRNA) strands, which have complementary nucleotide sequences to a targeted messenger RNA (mRNA) molecule. siRNAs are short, single-stranded nucleic acid molecules capable of inhibiting or down-regulating gene expression in a sequence-specific manner; see, for example, Zamore et al., Cell 101:25 33 (2000); Bass, Nature 411:428-429(2001); Elbashir et al., Nature 411:494-498 (2001); and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. Methods of preparing a siRNA molecule for use in gene silencing are described in U.S. Pat. No. 7,078,196, which is hereby incorporated by reference. Generally, one would prepare siRNA molecules that will specifically target one or more mRNAs without cross-reacting with other polynucleotides.

In some embodiments, the nucleic acid molecule encoded by a polynucleotide described herein is a microRNA (miRNA). miRNAs are non-coding sequences 20-25 nucleotides in length that play a vital role in the regulation of gene expression as they inhibit translation of their target mRNAs. miRNAs can control cell proliferation, differentiation, apoptosis, tumor formation, and drug susceptibility.

In some embodiments, an inducible promoter is activated by an electromagnetic wave. The common designations for electromagnetic waves are: radio waves, microwaves, infrared, visible light, ultraviolet, x-rays, and gamma rays. In some embodiments, the inducible promoter comprises an electromagnetic response element. In some embodiments, a promoter activated by an electromagnetic wave is a heat-inducible promoter. Heat inducible promoters include, but are not limited to HSP70 promoters, HSP90 promoters, HSP60 promoters, HSP27 promoters, HSP25 promoters, ubiquitin promoters, growth arrest or DNA Damage gene promoters, etc. See, e.g., U.S. Pat. Nos. 7,186,698; 7,183,262; and 7,285,542; See also I. Bouhon et al. Cytotechnology 33: 131-137 (2000) (gad 153 promoter). In some embodiments, heating is carried out by ultrasound, radiofrequency, laser, microwave, or water bath. Thus for deep tissue (e.g., located in the brain or other internal organ) the localized or selected heating may be carried out invasively or non-invasively. Suitable alternatives include, but are not limited to, a catheter with a heat tip, a catheter with an optical guide through which light or laser light beam can be directed (e.g., an infrared light) and by focused ultrasound (which can be delivered by any of a variety of different types of apparatus; see, e.g., U.S. Pat. Nos. 5,928,169; 5,938,608; 6,315,741; 6,685,639; 7,377,900; 7,510,536; 7,520,856; 8,343,050). The extent to which the selected tissue is heated will depend upon factors such as the choice of particular promoter, the duration of heating, and the tissue chosen for heating, but in general may be up to about 1 or 2 degrees centigrade to 5 or 6 degrees centigrade, for 1, 5, 10, or 15 minutes, or more. In some embodiments, heat activation is controlled by MRI directed ultrasound.

In some embodiments, optogenetic controlling is used to activate or inactivate an inducible promoter as described in (see, e.g., US Publication No. US20190119331). In some embodiments, the promoter is light inducible. For example, a blue-light sensing protein YFI which phosphorylates FixJ in the absence of light yielding a cascade signal to repress gene expression. When blue-light is present, the gene is expressed (see e.g., Addgene plasmid #43796).

In some embodiments, induction of gene expression is controlled using a high concentration sugar mixture (see, e.g., US Publication No. US20180148683).

Exemplary promoters include, but are not limited to, SFFV, isopropyl-beta-Isopropyl-beta-D-thiogalactopyranoside (IPTG)-regulated promoter, T7 RNA polymerase promoter, T3 RNA polymerase promoter, lactose-induced promoter, steroid-regulated promoter.

### 3. Cell Death-Inducing Polypeptides

In some embodiments, an engineered tissue construct described herein comprises at least one cell population engineered to express a cell death-inducing polypeptide. In some embodiments, a genetic construct encoding a cell death-inducing polypeptide is engineered into the cell or population of cells. In some embodiments, the cell death-inducing polypeptide is self-activating. In some embodiments, the cell death-inducing polypeptide is inducible.

In some embodiments, the cell death-inducing polypeptide is inactive and is constitutively expressed in a cell or population of cells. In some embodiments, the cell death-inducing polypeptide is operably linked to an inducible element to generate a fusion protein, wherein activation of the inducible element (e.g., by dimerization) activates the cell death-inducing polypeptide. In some embodiments, the cell death-inducing polypeptide is encoded by a nucleic acid sequence operably linked to an inducible promoter, such as those described herein. In some embodiments, the cell death-inducing polypeptide is expressed upon activation of the inducible promoter.

Exemplary cell death-inducing polypeptides include, but are not limited to, Casp2, Casp3, Casp8, Casp9, Casp10, p53, BAX, DFF40, HSV-TK, and cytosine deaminase proteins.

In some embodiments, the cell death-inducing polypeptide reduces, prevents, and/or eliminates the growth and/or survival of a senescent cell, such as, for example, by inducing cell death in the senescent cell via a cellular process including apoptosis. In some embodiments, cell death is induced via cellular processes including, but not limited to, necrosis/necroptosis, autophagic cell death, endoplasmic reticulum-stress associated cytotoxicity, mitotic catastrophe, paraptosis, pyroptosis, pyronecrosis, and entosis.

In some embodiments, the cell death-inducing polypeptide is a Caspase. Caspases are central components of the machinery for apoptosis. Apoptosis, or programmed cell death, plays a central role in the development and homeostasis of multicellular organisms (Jacobson, et al., Cell, 88, 347-354 (1997)). Fourteen distinct mammalian Caspases have been identified so far, with at least 7 of these identified as playing important roles during apoptosis (Shi, Mol Cell, 9, 459-470 (2002)), namely Caspases 2, 3, 6, 7, 8, 9 and 10.

Caspases involved in apoptosis are generally divided into two categories, the initiator Caspases, which include without limitation Caspase 1, 8, 9, and 10, and the effector Caspases which include without limitation Caspase 3, 6, and 7. An initiator Caspase is generally characterized by an extended N-terminal prodomain (>90 amino acids) important for its function, whereas an effector Caspase contains 20-30 residues in its prodomain sequence. Caspases are produced in cells as catalytically inactive zymogens and must undergo proteolytic activation during apoptosis. The activation of an effector Caspase (e.g. Caspase 3) is performed by an initiator Caspase (e.g. Caspase 8 or 9) through cleavage at specific internal aspartate residues that separate the large and small subunits. In contrast, the initiator Caspases are autoactivated. As this activation triggers a cascade of downstream Caspase activation, it is tightly regulated and requires the assembly of a multicomponent complex termed apoptosome (Bao and Shi, Cell Death Differ, 14, 56-65 (2007)). The initiator Caspases contain one of two protein-protein interaction motifs, the CARD (Caspase recruitment domain) or the DED (death effector domain). These motifs interact with similar motifs present on oligomerized adaptor proteins, bringing multiple initiator Caspase molecules into close proximity and facilitating their autoactivation (Shi, Mol Cell, 9, 459-470 (2002)).

The functional Caspase unit is a homodimer, with each monomer comprising a large 20 kDa and a small 10 kDA subunit. Homodimerization is mediated by hydrophobic interactions, with 6 antiparallel beta-strands from each catalytic subunit forming a single contiguous 12-stranded beta-sheet. Several alpha-helices and short beta-strands are located on either side of the central beta-sheet, giving rise to a globular fold. The active sites, formed by four protruding loops from the scaffold, are located at two opposite ends of the beta-sheet (Shi, Mol Cell, 9, 459-470 (2002)). Once activated the effector Caspases are responsible for the proteolytic cleavage of a broad spectrum of cellular targets, leading ultimately to cell death.

In some embodiments, the cell death-inducing polypeptide is a fusion protein comprising a Caspase domain or a functionally active variant thereof and a ligand binding domain. Such fusion proteins are described briefly herein, and in U.S. Pat. No. 8,530,168, hereby incorporated by reference.

In some embodiments, the ligand binding domain is one described herein, e.g., an FKBP-derived dimeriser (CID) domain. Forced oligomerisation leads to Caspase activation. In some embodiments, a CID domain is fused onto the N-terminal end of a Caspase or Caspase domain(s).

In some embodiments, a first component of the fusion protein is a functionally active variant of a Caspase domain. In some embodiments, a functional active variant has a biological activity similar to that displayed by the Caspase domain from which it is derived, including the ability to induce apoptosis. Generation and testing of Caspase fragments and variants is described in U.S. Pat. No. 8,530,168. In some embodiments, a second component of the fusion protein of the invention is a ligand binding domain as described herein.

Methods for measuring apoptosis and cell death are known to those of ordinary skill in the art. For example, cell death may be measured by Giemsa staining, trypan blue exclusion, acridine orange/ethidium bromide (AO/EB) double staining for fluorescence microscopy and flow cytometry, propidium iodide (PI) staining, annexin V assay, TUNEL assay, DNA ladder, LDH activity, and MTT assay. Cell death due to induction of apoptosis may be measured by observation of morphological characteristics including cell shrinkage, cytoplasmic condensation, chromatin segregation and condensation, membrane blebbing, and the formation of membrane-bound apoptotic bodies. Cell death due to induction of apoptosis may be measured by observation of biochemical hallmarks including internucleosomal DNA cleavage into oligonucleosome-length fragments. Additional methods include flow cytometry analysis using SYTOX and AnnexinV. Traditional cell-based methods of measuring cell death due to induction of apoptosis include light and electron microscopy, vital dyes, and nuclear stains. Biochemical methods include DNA laddering, lactate dehydrogenase enzyme release, and MTT/XTT enzyme activity.

In some embodiments, a cell death-inducing polypeptide eliminated at least one cell population from the engineered tissue construct. In some embodiments, the cell death-inducing polypeptide eliminates the cell population expressing the cell death-inducing polypeptide. In some embodiments, the cell death-inducing polypeptide eliminates at least one cell population that does not express the cell death-inducing polypeptide.

In some embodiments, a cell death-inducing polypeptide forms apoptotic bodies in the engineered tissue construct. Apoptotic bodies have been found to provide long-term immune tolerance through the generation of alloantigen-specific Treg cells (Kuang, R. et al. Cell & Bioscience, 2015, Vol 5 (27)). Immune tolerance is critical for successful transplantation. Accordingly, in some embodiments, formation of apoptotic bodies in vitro or in vivo allows for successful implantation of an engineered tissue construct.

### 4. Tissue Expansion and Organo Genesis Inducing Molecules

In some embodiments, an engineered tissue construct described herein comprises at least one cell population comprising a biological molecule (e.g., nucleic acid molecule or polypeptide of interest) that impacts tissue organogenesis and/or expansion.

In some embodiments, tissue organogenesis is the formation of a tissue from an engineered tissue construct described herein. In some embodiments, the engineered tissue construct comprises a stem cell or precursor cell that differentiates to form a tissue. In some embodiments, an engineered tissue construct comprising a stem cell or precursor cell comprises at least one cell population comprising an inducible differentiation factor (i.e., a polynucleotide encoding a differentiation factor operably linked to an inducible element or a polynucleotide comprising an inducible promoter operably linked to a nucleotide sequence encoding a polypeptide or a nucleic acid molecule capable of inducing differentiation).

In some embodiments, the biological molecule induces differentiation of at least one cell population. For example, in embodiments wherein the tissue construct comprises a stem cell or precursor cell, such cells can be differentiated based on expression and/or activation of a biological molecule. Differentiation factors are known to those of skill in the art, and include for example, cytokines and growth factors such as fibroblast growth factor and transforming growth factor beta.

In some embodiments, cell differentiation is measured by analyzing the expression level of at least one cell marker known to be expressed on a differentiated cell and not expressed on a stem cell or precursor cell. In some embodiments, cell differentiation is measured by analyzing the expression level of at least one cell marker known to be expressed on a stem cell or precursor cell and not expressed on a differentiated cell.

In some embodiments, tissue organogenesis comprises cell proliferation of at least one cell population within the engineered tissue construct. In some embodiments, the biological molecule induces cell proliferation of at least one cell population within the tissue construct. Methods for measuring cell proliferation are known to those of skill in the art and include, but are not limited to, measuring the number of cells and/or measuring proliferation markers. In some embodiments, proliferation is measured using cytoplasmic proliferation dyes, in which a cell permeable fluorescent chemical binds to cytosolic components and is diluted in half every cell division. Such dyes can be used in vitro and in vivo. Examples include measuring carbozyfluorescein diacetate (CFSE). In some embodiments, proliferation is measured by quantifying the level of a cell-cycle associated protein. Cell-cycle associated proteins include, but are not limited to Ki67, Histone H3, proliferating cell nuclear antigen (PCNA), and minichromosome maintenance (MCM). In some embodiments, proliferation is measured using nucleoside-analog incorporation assays. Examples of incorporation assays induce the tritiated thymidine incorporation assay and the BrdU incorporation assay. In some embodiments, proliferation is indirectly measured using metabolic activity assays. Examples include but are not limited to the tetrazolium assay and resazurin reduction assay.

Multiple techniques are applicable to measure any of the cell proliferation proteins described herein. Examples of techniques include, flow cytometry, western blot analysis, and tissue microscopy. Manual methods for determining cell proliferation may be used including counting total cell number.

In some embodiments, the biological molecule induces cell-cell interactions among the same cell type. In some embodiments, the biological molecule induces cell-cell interactions between different cell types.

In some embodiments, the biological molecule induces overall expansion of the tissue construct. In some embodiments, expansion of the tissue construct includes an increase in size, volume, and/or area. In some embodiments, expansion of the tissue construct includes an increase in weight.

In some embodiments, the biological molecule is a growth factor. In some embodiments, the growth factor is selected from Wnt2, EGF, HGF, FGF (e.g., FGF7 or FGF10), RSP03, PGE2, and CSF1. In some embodiments, the biological molecule is an angiogenic factor. In some embodiments, the angiogenic factor is selected from GATA4, IL-8, VEGF, Ang-2, IL-6, DLL4 and ID-1. In some embodiments, the biological molecule is expressed using an inducible promoter or inducible element. In some embodiments, the molecule is expressed during tissue development and is inhibited after establishment of the engineered tissue construct.

In some embodiments, tissue organogenesis occurs in vitro. In some embodiments, tissue organogenesis occurs in vivo. In some embodiments, differentiation of a stem cell or precursor cell occurs in vivo. In some embodiments, differentiation of a stem cell or precursor cell occurs in vitro. In some embodiments, cell proliferation of at least one cell population occurs in vitro. In some embodiments, cell proliferation of at least one cell population occurs in vivo. In some embodiments, the engineered tissue construct expands in vitro. In some embodiments, the engineered tissue construct expands in vivo.

### 5. Multiplexed Tissue Constructs

In some embodiments, the engineered tissue construct comprises more than one genetic construct described herein to form a multiplexed engineered tissue construct. Multiplexing involves introducing multiple gene edits within one cell type, or simultaneous gene-editing in multiple cell types.

In some embodiments, the more than one genetic construct is provided in two distinct cell populations. In some embodiments, the two distinct cell populations comprise the same genetic construct. For example, in some embodiments, two distinct cell populations (e.g., stromal cells and endothelial cells) each comprise a genetic construct comprising a polynucleotide encoding a cell death-inducing polypeptide.

In some embodiments, the more than one genetic construct is provided within the same cell population. In some embodiments, the more than one genetic constructs within the same cell population are different. For example, in some embodiments, one cell population comprises a genetic construct comprising a polynucleotide encoding a growth factor, and a genetic construct comprising a polynucleotide encoding a cell death-inducing polypeptide.

In some embodiments, the more than one genetic construct is under the control of the same stimulus (e.g., a biological molecule or small molecule). In some embodiments, the more than one genetic constructs are under the control of different stimuli (e.g., different biological molecules or small molecules (i.e. orthogonal switches)).

In some embodiments, any engineered tissue construct described herein with two or more cell types is engineered to have one or more orthogonal switches. For example, in some embodiments an engineered tissue construct comprises a non-parenchymal cell with insertion of a chemically-inducible iCasp9 and a parenchymal cell with insertion of a polynucleotide expressing a gene of interest (e.g., a growth factor) under control of an inducible promoter.

In some embodiments, the multiplexed tissue construct has two, three, four, five, or more orthogonal switches. In some embodiments, the orthogonal switches control one, two, three, four, five, or more gene-edits. In some embodiments, the orthogonal switches are within the same cell population. In some embodiments, the orthogonal switches are within different cell populations.

In some embodiments, a cell or cell population comprises more than one polynucleotide encoding a polypeptide of interest under control of an inducible element that is activated upon interaction with a biological molecule or small molecule. In some embodiments, the inducible elements are the same. In some embodiments, the inducible elements are orthogonal.

In some embodiments, a cell or cell population comprise more than one polynucleotide encoding a polypeptide of interest under control of an inducible promoter. In some embodiments, the inducible promoters are the same. In some embodiments, the inducible promoters are orthogonal.

In some embodiments, a cell or cell population comprises (i) a polynucleotide encoding a polypeptide of interest operably linked to an inducible element and (ii) a polynucleotide comprising an inducible promoter operably linked to a nucleotide sequence encoding a polypeptide or a nucleic acid molecule of interest.

In some embodiments the orthogonal switches are targeted sequentially. In some embodiments, the orthogonal switches are targeted simultaneously. In some embodiments, the one or more orthogonal switches are genetic (e.g. an inducible promoter). In some embodiments, the one or more orthogonal switches are controlled by a small molecule (e.g. chemically inducible dimerization). In some embodiments, the orthogonal switches are activated by any of the methods described herein.

In some embodiments, the orthogonal switches are activated for the same length of time. In some embodiments, the orthogonal switches are activated for different lengths of time. In some embodiments, the orthogonal switches are activated and turned off one or more times.

### Cells

In some embodiments, the engineered tissue construct described herein comprises at least one population of cells. In some embodiments, the engineered tissue construct comprises at least two populations of cells, in which the two populations of cells are different. In some embodiments, the engineered tissue construct comprises at least one population of parenchymal cells.

In some embodiments, the engineered tissue construct comprises parenchymal cells. In some embodiments, the engineered tissue construct comprises non-parenchymal cells. In some embodiments, the engineered tissue construct comprises parenchymal and non-parenchymal cells. In some embodiments, the engineered tissue construct comprises more than one cell population of non-parenchymal cells. In some embodiments, the engineered tissue construct comprises more than one cell population of parenchymal cells. In some embodiments, the engineered tissue construct comprises one cell population of parenchymal cells and two cell populations of non-parenchymal cells.

Parenchymal cells are obtained from a variety of sources including, but not limited to, liver, skin, pancreas, neuronal tissue, muscle (e.g., heart and skeletal), and the like. Parenchymal cells are obtained from parenchymal tissue using any one of a host of art-described methods for isolating cells from a biological sample, e.g., a human biological sample. In some embodiments, parenchymal cells. e.g., human parenchymal cells, are obtained by biopsy or from cadaver tissue. In some embodiments, parenchymal cells are derived from lung, kidney, nerve, heart, fat, bone, muscle, thymus, salivary gland, pancreas, adrenal, spleen, gall bladder, liver, thyroid, parathyroid, small intestine, uterus, ovary, bladder, skin, testes, prostate, or mammary gland.

In some embodiments, constructs contain human parenchymal cells optimized to maintain the appropriate morphology, phenotype and cellular function conducive to use in the methods of the disclosure. In some embodiments, primary human parenchymal cells are isolated and/or pre-cultured under conditions optimized to ensure that the parenchymal cells of choice (e.g., hepatocytes) initially have the desired morphology, phenotype and cellular function and, thus, are poised to maintain said morphology, phenotype and/or function in the constructs, and in vivo upon implantation to create the engineered tissue construct described herein.

Cells useful in the constructs and methods of the disclosure are available from a number of sources including commercial sources. For example, in some embodiments, hepatocytes are isolated by conventional methods (Berry and Friend, 1969, J. Cell Biol. 43:506-520) and adapted for human liver biopsy or autopsy material. Methods for obtaining cells, including perfusion methods or other methods are known in the art, such as those described in U.S. Pat. Pub. No. 20060270032.

In some embodiments, the cell types seeded in the tissue construct includes, but is not limited to, hepatocytes, liver progenitor cells, pancreatic cells (alpha, beta, gamma, delta), enterocytes, renal epithelial cells, astrocytes, muscle cells, brain cells, neurons, glia cells, astrocytes, respiratory epithelial cells, lymphocytes, erythrocytes, blood-brain barrier cells, kidney cells, and other parenchymal cell types known in the art, cancer cells, normal or transformed fibroblasts, oval cells, adipocytes, osteoblasts, osteoclasts, myoblasts, beta-pancreatic islets cells, stem cells (e.g., embryonic stem cells, hematopoietic stem cells, mesenchymal stem cells, endothelial stem cells, etc.), cells described in U.S. patent application Ser. No. 10/547,057 paragraphs 0066-0075, myocytes, keratinocytes, any other non-parenchymal cell types known in the art, and indeed any cell type that adheres to a substrate.

In some embodiments, the cells are mammalian cells. In some embodiments, the mammalian cells are derived from two different species (e.g., humans, mice, rats, primates, pigs, and the like). In some embodiments, the cells are primary cells, or they are derived from an established cell-line. In some embodiments, cells are from multiple donor types, progenitor cells (e.g., liver progenitor cells), or tumor cells, and the like. In some embodiments, the cells are freshly isolated cells (for example, encapsulated within 24 hours of isolation), e.g., freshly isolated hepatocytes from cadaveric donor livers. Although any combination of cell types that promotes maintenance of differentiated function of the parenchymal cells can be used (e.g., parenchymal and one or more populations of non-parenchymal cells, e.g., stromal cells), an exemplary combination of cells for producing the constructs include, without limitation: fibroblasts and endothelial cells. In some embodiments, the combination of cells is fibroblasts and endothelial cells. In some embodiments, combinations include, without limitation, (a) human hepatocytes (e.g., primary hepatocytes) and fibroblasts (e.g., normal or transformed fibroblasts, including, for example, non-human transformed fibroblasts); (b) hepatocytes and at least one other cell type, particularly liver cells, such as Kupffer cells, Ito cells, endothelial cells, and biliary ductal cells; and (c) stem cells (e.g., liver progenitor cells, oval cells, hematopoietic stem cells, embryonic stem cells, and the like) and a non-parenchymal cell population, for example, stromal cells (e.g., fibroblasts). In some embodiments it desirable to include immune cells in the constructs, e.g., Kupffer cells, macrophages, B-cells, dendritic cells, etc. In some embodiments, the combination of cells includes human hepatocytes, fibroblast, and endothelial cells.

In some embodiments, hepatocytes are derived from any source known in the art, e.g., primary hepatocytes, progenitor-derived, ES-derived, induced pluripotent stem cells (iPS-derived), etc. In some embodiments, hepatocytes useful in the constructs and methods described herein are produced by the methods described in Takashi Aoi et al., Science 321 (5889): 699-702; U.S. Pat. Nos. 5,030,105; 4,914,032; 6,017,760; 5,112,757; 6,506,574; 7,186,553; 5,521,076; 5,942,436; 5,580,776; 6,458,589; 5,532,156; 5,869,243; 5,529,920; 6,136,600; 5,665,589; 5,759,765; 6,004,810; U.S. Pat. application Ser. Nos. 11/663,091; 11/334,392; 11/732,797; 10/810,311; and PCT application PCT/JP2006/306783, all of which are incorporated herein by reference in their entirety.

In some embodiments, the tissue construct comprises endothelial cells. In some embodiments, the endothelial cells are adult vein endothelial cells, adult artery endothelial cells, embryonic stem cell-derived endothelial cells, iPS-derived endothelial cells, umbilical vein endothelial cells, umbilical artery endothelial cells, endothelial progenitors cells derived from bone marrow, endothelial progenitors cells derived from cord blood, endothelial progenitors cells derived from peripheral blood, endothelial progenitors cells derived from adipose tissues, endothelial cells derived from adult skin, or a combination thereof. In some embodiments, the umbilical vein endothelial cells are human umbilical vein endothelial cells (HUVEC).

In some embodiments, the cells of the tissue construct are fibroblasts and/or fibroblast-like cells. In some embodiments, the fibroblasts are human foreskin fibroblasts, human embryonic fibroblasts, mouse embryonic fibroblasts, skin fibroblasts cells, vascular fibroblast cells, myofibroblasts, smooth muscle cells, mesenchymal stem cells (MSCs)-derived fibroblast cells, or a combination thereof. In some embodiments the fibroblasts are normal human dermal fibroblasts (NHDFs). In some embodiments, the fibroblasts are growth-arrested human dermal fibroblasts (HDFs).

In some embodiments, the cells of the tissue construct are derived from endothelial cells and fibroblasts. In some embodiments, the cells of the tissue construct are derived from fibroblasts and hepatocytes. In some embodiments, the cells of the tissue construct are derived from endothelial cells and hepatocytes. In some embodiments, the cells of the tissue construct are derived from hepatocytes, endothelial cells and fibroblasts. In some embodiments, the cells of the tissue construct are derived from HUVECs and HDFs. In some embodiments, the cells of the tissue construct are derived from HDFs and primary human hepatocytes. In some embodiments, the cells of the tissue construct are derived from HUVECs and primary human hepatocytes. In some embodiments, the cells of the tissue construct are derived from HUVECs, HDFs, and primary human hepatocytes.

### Methods for Engineering Cells to Express Genetic Constructs

A variety of different methods known in the art can be used to introduce any of the nucleic acids or polynucleotides disclosed herein into any cell described herein. Non-limiting examples of methods for introducing a nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce nucleic acids into cells and/or to introduce other genetic modifications.

Methods for transduction of inducible genes into the cells described herein are known by those of skill in the art. For example, individual plasmids of interest are co-transfected into HEK-293T cells with pVSVG, pRSV-REV, and pMDLg/pRRE using the calcium phosphate transfection method. Supernatants containing the assembled viruses are collected precipitated. The cells of interest (for example, HDFs and HUVECs) are transduced in growth media overnight with the appropriate lentiviral titers.

In some embodiments, a cell is transduced with lentivirus encoding a polynucleotide of interest. In some embodiments, a cell is transduced with a bidirectional expression cassette encoding iCasp9 and GFP genes (#15567 pMSCV-F-del Casp9.IRES.GFP; cloned to a lentivirus plasmid backbone with an SFFV promoter).

In some embodiments, gene-editing is used to delete a gene or genetic locus of interest. Non-limiting examples of methods for genetic deletion include: CRISPR/Cas9 gene editing technology, zinc finger nuclease technology, and transcription activator-like effector nucleases (TALE'S).

### Biocompatible Scaffolds and Substrates

In some embodiments, a tissue construct described herein comprises a bioscaffold (i.e., a biocompatible scaffold). Bioscaffolds are natural or artificially derived three-dimensional structures utilized in tissue engineering to promote cell and tissue growth. In some embodiments, the tissue construct described herein is seeded in a biocompatible scaffold. In some embodiments, the biocompatible scaffold is a hydrogel. A hydrogel is characterized by a high permeability for exchange of nutrients necessary for cell proliferation. The physical properties of hydrogels are similar to native tissue. In some embodiments, hydrogels are used to encapsulate cells in the hydrogel matrix formed upon gelation.

Generally, a hydrogel is formed by using at least one, or one or more types of hydrogel precursor, and setting or solidifying the one or more types of hydrogel precursor in an aqueous solution to form a three-dimensional network, wherein formation of the three-dimensional network may cause the one or more types of hydrogel precursor to gel.

In some embodiments, the hydrogel precursor comprises a natural polymer. The natural polymer may form a three-dimensional network in an aqueous medium to form a hydrogel. A “natural polymer” refers to a polymeric material that is found in nature. In some embodiments, the natural polymer includes, but is not limited to, polysaccharide, glycosaminoglycan, protein, peptide and polypeptide.

Polysaccharides are carbohydrates which are capable of hydrolyzing to two or more monosaccharide molecules. They may contain a backbone of repeating carbohydrate i.e. sugar unit. Examples of polysaccharides include, but are not limited to, alginate, agarose, chitosan, dextran, starch, and gellan gum. In some embodiments the natural polymer is any of alginate, agarose, chitosan, dextran, starch, and gellan gum.

Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine. In some embodiments, the natural polymer is any of hyaluronic acid, chondroitin sulfate, dermatin sulfate, keratin sulfate, dextran sulfate, heparin sulfate, heparin, glucuronic acid, iduronic acid, galactose, galactosamine, and glucosamine.

In some embodiments, a hydrogel precursor includes a hydrophilic monomer. As used herein, a hydrophilic monomer refers to any monomer which, when polymerized, yields a hydrophilic polymer capable of forming a hydrogel when contacted with an aqueous medium such as water. In some embodiments, a hydrophilic monomer contains a functional group in the polymer backbone or as lateral chains.

Examples of hydrophilic monomers include, but are not limited to, hydroxyl-containing monomers such as 2-hydroxyethyl methacrylate, 2-hydroxyethyl acrylate, 2-hydroxyethyl methacrylamide, 2-hydroxyethyl acrylamide, N-2-hydroxyethyl vinyl carbamate, 2-hydroxyethyl vinyl carbonate, 2-hydroxypropyl methacrylate, hydroxyhexyl methacrylate and hydroxyoctyl methacrylate; carboxyl-containing monomers such as acrylic acid, methacrylic acid, itaconic acid, fumaric acid, crotonic acid, maleic acid and salts thereof, esters containing free carboxyl groups of unsaturated polycarboxylic acids, such as monomethyl maleate ester, monoethyl maleate ester, monomethyl fumarate ester, monoethyl fumarate ester and salts thereof; amide containing monomers such as (meth)acrylamide, crotonic amide, cinnamic amide, maleic diamide and fumaric diamide; thiol-containing monomers such as methanethiole, ethanethiol, 1-propanethiol, butanethiol, tert-butyl mercaptan, and pentanethiols; sulfonic acid-containing monomers such as p-styrenesulfonic acid, vinylsulfonic acid, p-a-methylstyrene sulfonic acid, isoprene sulfonide and salts thereof.

In some embodiments, a hydrogel precursor includes a hydrophilic polymer. In some embodiments, the hydrophilic polymer is a polymer that is made up of any one of the above-mentioned hydrophilic monomer, and which is formed from any reaction such as, but not limited to, free radical polymerization, condensation polymerization, anionic or cationic polymerization, or step growth polymerization. For example, a hydrophilic monomer such as ethylene glycol may undergo anionic or cationic polymerization, depending on the type of catalyst used, to form poly(ethylene glycol) which is a hydrophilic polymer. In some embodiments, the hydrophilic polymer is obtained by chemical modification of an existing polymer. For example, a functional group is added or altered on polymeric chains such that the resultant polymer is made hydrophilic.

In some embodiments, a hydrogel precursor includes a hydrophilic copolymer. In some embodiments, the hydrophilic copolymer is formed from a hydrophilic polymer and a monomer of which is any of hydrophilic, hydrophobic or amphiphilic. In some embodiments, a hydrophilic copolymer is formed from a hydrophilic monomer and a polymer of which is any of hydrophilic, hydrophobic or amphiphilic. For example, a hydrophobic monomer reacts with a functional group present on a hydrophilic polymer to form a hydrophilic copolymer.

The one of more types of hydrogel precursors may set or solidify in an aqueous medium to form a three-dimensional network, wherein formation of the three-dimensional network can cause the one or more types of hydrogel precursor to gel. For example, a hydrogel is formed by physical bonding such as self-assembly, or chemical bonding such as cross-linking, of one or more types of hydrogel precursors in an aqueous medium.

In some embodiments, a hydrogel is formed by self-assembly of one or more types of hydrogel precursors in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains may aggregate or coagulate in an aqueous medium, which may form a three-dimensional network, thereby entrapping molecules of water to form a hydrogel.

In some embodiments, a hydrogel is formed by chemical bonding between one or more types of hydrogel precursors in an aqueous medium. For example, when the hydrogel precursor is a hydrophilic polymer, the polymeric chains may be cross-linked using a suitable cross-linking agent to form a three-dimensional network, which entraps water molecules to form a hydrogel. Methods for chemical cross-linking are carried out by reactions, such as any-one of free radical polymerization, condensation polymerization, anionic or cationic polymerization, or step growth polymerization.

The term “cross-linking agent” refers to an agent which induces cross-linking. In some embodiments, the cross-linking agent is any agent that is capable of inducing a chemical bond between adjacent polymeric chains. For example, in some embodiments, the cross-linking agent is a chemical compound. Examples of chemical compounds that act as cross-linking agent include, but are not limited to, dextran dialdehyde, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisacrylamide, N,N′-methylenebis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, or mixtures thereof.

In some embodiments, the cross-linking agent comprises or consists of dextran dialdehyde. When dextran dialdehyde is added to a hydrogel precursor such as gelatin, for example, covalent bonds are formed between the aldehyde group of the dextran dialdehyde and the amino group of gelatin, thereby cross-linking the gelatin to form dextran dialdehyde-crosslinked gelatin hydrogel via chemical bonding.

In some embodiments, the hydrogel precursors are used as cross-linking agents, and do not require addition or use of a separate cross-linking agent.

In some embodiments, the cross-linking agent is in the form of an electromagnetic wave. Therefore, in some embodiments, cross-linking is carried out using an electromagnetic wave, such as gamma or ultraviolet radiation, which may cause the polymeric chains to cross-link and form a three-dimensional matrix, thereby entrapping water molecules to form a hydrogel. Therefore, choice of cross-linking agent is dependent on the type of polymeric chain and functional group present, and a person skilled in the art would be able to choose the appropriate type of cross-linking agent accordingly.

Cross-linking induced by cross-linking agents is used to vary the degradation time of the hydrogel used for the composite of the present invention. The final degradation rate of hydrogel is measured using techniques known in the art. Most commonly, the degradation rate of a hydrogel is determined by measuring dry weight loss of the hydrogel over time.

In some embodiments, the biocompatible scaffold comprises or consists of one or more synthetic or natural hydrophilic polymers. For example, in some embodiments, the hydrogel is made of one or more materials selected from the group consisting of polysaccharides, proteins, polyethylene glycol, polylactic acid, polycaprolactone, polyglycolide, and combinations thereof. In some embodiments, the hydrogel comprises or consists of one or more compounds selected from the group consisting of dextran, chitosan, hyaluronic acid, gelatin, dextran dialdehyde-crosslinked gelatin, collagen, aminated hyaluronic acid, hyaluronic acid-g-poly(N-isopropylacrylamide), chitosan-hyaluronic acid, laminin, elastin, alginate, fibronectin, polyethylene glycol-fibrinogen, and derivatives thereof.

In some embodiments, either one of or both the hydrogel and the plurality of fibers comprise or consist of a biodegradable polymer. The term “biodegradable” refers to a substance which is broken down by microorganisms, or which spontaneously breaks down over a relatively short time (within 2-15 months) when exposed to environmental conditions commonly found in nature. For example, gelatin may be degraded by enzymes which are present in the body. In some embodiments, the scaffold is degradable upon exposure to environmental conditions. For example, in some embodiments, the scaffold is degraded by the presence of hydrolytic enzymes, presence of proteasomal enzymes, pH lower than 5 and reducing conditions.

Examples of biodegradable polymers include, but are not limited to, polymers and oligomers of glycolide, lactide, polylactic acid, polyesters of a-hydroxy acids, including lactic acid and glycolic acid, such as the poly(a-hydroxy) acids including polyglycolic acid, poly-DL-lactic, poly-L-lactic acid, and terpolymers of DL-lactide and glycolide; e-caprolactone and e-caprolactone copolymerized with polyesters; polylactones and polycaprolactones including poly(e-caprolactone), poly(8-valerolactone) and poly (gamma-butyrolactone); polyanhydrides; polyorthoesters; other hydroxy acids; polydioxanone; and other biologically degradable polymers that are non-toxic or are present as metabolites in the body. Examples of polyaminoacids include, but are not limited to, polylysine (PLL), poly L-aspartic acid, poly L-glutamic acid, and styrene-maleic acid anhydride copolymer. Examples of derivatives of polyethylene glycol includes, but are not limited to, poly(ethylene glycol)-di-(ethylphosphatidyl(ethylene glycol)) (PEDGA), poly(ethylene glycol)-co-anhydride, poly(ethylene glycol)co-lactide, poly(ethylene glycol)-co-glycolide and poly (ethylene glycol)-co-orthoester. Examples of acrylamide polymers include, but are not limited to, polyisopropylacrylamide, and polyacrylamide. Examples of acrylate polymers include, but are not limited to, diacrylates such as polyethylene glycol diacrylate (PEGDA), oligoacrylates, methacrylates, dimethacrylates, oligomethoacrylates and PEG-oligoglycolylacrylates. Examples of carboxy alkyl cellulose include, but are not limited to, carboxymethyl cellulose and partially oxidized cellulose.

In some embodiments, degradation of the fiber-reinforced hydrogel composite takes place over a time period ranging from a few seconds to a few days or months. The time period required for the fiber-reinforced hydrogel composite to degrade is dependent on a few parameters, for example, constituent of the fiber-reinforced hydrogel composite, such as type of hydrogel and/or fibers used and water content of the hydrogel, degree of cross-linking, temperature, pH, amount of aqueous medium present, and pressure during gelation. This period may be extended by varying the degree of cross-linking as described above.

In some embodiments, the hydrogel comprises or consists of dextran dialdehyde-crosslinked gelatin, in which gelatin is used as the hydrogel precursor and dextran dialdehyde is used as the cross-linking agent. The term “gelatin” as used herein refers to protein substances derived from collagen. In the context of the present invention, “gelatin” also refers to equivalent substances such as synthetic analogues of gelatin. Generally, gelatin is classified as alkaline gelatin, acidic gelatin, or enzymatic gelatin. Alkaline gelatin may be obtained from the treatment of collagen with a base such as sodium chloride. Acidic gelatin may be obtained from the treatment of collagen with an acid such as hydrochloric acid. Enzymatic gelatin may be obtained from the treatment of collagen with an enzyme such as hydrolase. As gelatin may be a form of hydrogel, factors that affect degradation behavior of hydrogels as mentioned herein may apply to gelatin as well.

In some embodiments, the material of the hydrogel and the material of the fibers are the same. In such embodiments, the material of the hydrogel and the material of the fibers are selected such that the physical properties of the respective material are different. For example, the hydrogel material and the fiber material comprised in the fibrous component may have different elasticity and toughness.

In some embodiments, the material of the hydrogel and the material of the fibers are different. For example, in one specific embodiment, the composite comprises or consists of a hydrogel of dextran dialdehyde-crosslinked gelatin and fibers of bovine serum albumin.

In some embodiments, the composite comprises a plurality of layers of the hydrogel and/or a plurality of layers of the fibrous component. In other words, the composite may comprise a plurality of layers of the hydrogel and a layer of the fibrous component, or vice versa. Each of the plurality of layers of the hydrogel and/or the plurality of layers of the fibrous component may be the same or different, in terms of the thickness of the layers or the content of the layers. For example, the composite may comprise more than one layer of the hydrogel, or more than one layer of the fibrous component, or more than one layer of each of the hydrogel and fibrous component. In such embodiments, the composite may consist of alternating layers of the hydrogel and the fibrous component.

In some embodiments, a cell population is placed on a substrate. Various culture substrates can be used in the constructs of the disclosure. Such substrates include, but are not limited to, glass, polystyrene, polypropylene, stainless steel, silicon and the like. In some embodiments, the substrate is poly(methyl methacrylate). In some embodiments, the substrate is a polycarbonate, acrylic copolymer, polyurethane, aluminum, carbon or Teflon (polytetrafluoroethylene). The cell culture surface can be chosen from any number of rigid or elastic supports. For example, cell culture material can comprise glass or polymer microscope slides. In some embodiments, the substrate may be selected based upon a tissue's propensity to bind to the substrate. In some embodiments, the substrate may be selected based on the potential effect of the substrate on the tissue explant (e.g., electrical stimulation/resistivity, mechanical stimulation/stress).

The cell culture surface/substrate can be made of any material suitable for culturing mammalian cells. For example, the substrate can be a material that can be easily sterilized such as plastic or other artificial polymer material, so long as the material is biocompatible. In some embodiments, the substrate is any material that allows cells and/or tissue to adhere (or can be modified to allow cells and/or tissue to adhere or not adhere at select locations). Any number of materials can be used to form the substrate/surface, including but not limited to, polyamides; polyesters; polystyrene; polypropylene; polylacrylates; polyvinyl compounds (e.g., polyvinylchloride); polycarbonate; polytetrafluoroethylene (PTFE); nitrocellulose; cotton; polyglyolic acid (PGA); cellulose; dextran; gelatin; glass; fluoropolymers; fluorinated ethylene propylene; polyvinylidene; polydimethylsiloxane; and silicon substrates (such as fused silica, polysilicon, or single silicon crystals), and the like. Also, metals (e.g., gold, silver, titanium films) can be used.

In some embodiments, the substrate may be modified to promote cellular adhesion (e.g., coated with an adherence material). For example, a glass substrate may be treated with a protein (i.e., a peptide of at least two amino acids) such as collagen or fibronectin to assist cells of the tissue in adhering to the substrate. In some embodiments, a single protein is adhered to the substrate. In some embodiments, two or more proteins are adhered to the substrate. Proteins suitable for use in modifying the substrate to facilitate adhesion include proteins to which specific cell types adhere under cell culture conditions.

The type of adherence material(s) (e.g., ECM materials, sugars, proteoglycans, etc.) deposited on the substrate will be determined, in part, by the cell type or types in the tissue construct.

### 1. Bioactive Agent

In some embodiments, the biocompatible scaffold or substrate described herein comprises at least one bioactive agent. In some embodiments, the bioactive agent is a growth factor. In some embodiments, the bioactive agent is an extracellular matrix protein.

In some embodiments, the biocompatible scaffold or substrate comprises a vascular endothelial growth factor (VEGF). VEGF is a key protein in physiological angiogenesis (or neo-vascularization), or formation of new blood vessels. N. Ferrara et al., The biology of VEGF and its receptors, 9 Nat. Med. 669-676 (2003).

In some embodiments, the biocompatible scaffold or substrate comprises a fibroblast growth factor (FGF).

In some embodiments, the biocompatible scaffold or substrate comprises a bioactive agent selected from angiopoietins, extracellular matrix proteins (e.g., fibronectin, vitronectin, collagen), adhesion proteins, BMPs, TGFβ, SDFs, interleukins, interferons, CXCLs, and lipoproteins. In some embodiments, the bioactive agent is any protein having a positive charge.

In some embodiments, the bioactive agent improves the function of the biocompatible scaffold. For example, in some embodiments, a hydrogel comprising a bioactive agent enhances multicellular sprouting compared to a hydrogel lacking a bioactive agent.

In some embodiments, the scaffolding or substrate contains one or more bioactive substances. Examples of bioactive substance(s) include, but are not limited to, hormones, neurotransmitters, growth factors, hormone, neurotransmitter or growth factor receptors, interferons, interleukins, chemokines, cytokines, colony stimulating factors, chemotactic factors, extracellular matrix components, and adhesion molecules, ligands and peptides; such as growth hormone, parathyroid hormone (PTH), bone morphogenetic protein (BMP), transforming growth factor-α. (TGF α), TGFβ1, TGFβ2, fibroblast growth factor (FGF), granulocyte/macrophage colony stimulating factor (GMCSF), epidermal growth factor (EGF), platelet derived growth factor (PDGF), insulin-like growth factor (IGF), scatter factor/hepatocyte growth factor (HGF), fibrin, collagen, fibronectin, vitronectin, hyaluronic acid, an RGD-containing peptide or polypeptide, an angiopoietin and vascular endothelial cell growth factor (VEGF). In some embodiments, the tissue construct comprises a biologically effective amount of VEGF. In some embodiments, the tissue construct comprises at least one cell of one cell type and at least one bioactive agent. In some embodiments, the tissue construct is free from exogenous bioactive substances.

In some embodiments, the naturally-derived or synthetic scaffolding used to form the tissue construct can release bioactive substances compared to the scaffold. For example, naturally-derived or synthetic scaffolding used to form the pre-templated vessels and/or cell clusters or islands can release pro-angiogenic factors.

### 2. Adherence Material

In some embodiments, the biocompatible scaffold or substrate comprises an adherence material. The term “adherence material” is a material incorporated into a hydrogel or onto a substrate disclosed herein to which a cell or microorganism has some affinity, such as a binding agent. The material is incorporated, for example, into a hydrogel or onto a substrate prior to seeding with parenchymal and/or non-parenchymal cells. The material and a cell or microorganism interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, sugars, proteoglycans, or cellular receptors.

The type of adherence material(s) (e.g., ECM materials, sugars, proteoglycans etc.) will be determined, in part, by the cell type or types cultured. ECM molecules found in the parenchymal cell's native microenvironment are useful in maintaining the function of both primary cells, and precursor cells and/or cell lines. For example, hepatocytes are known to bind to collagen. Therefore, collagen is well suited to facilitate binding of hepatocytes. The liver has heterogeneous staining for collagen I, collagen III, collagen IV, laminin, and fibronectin. Hepatocytes also display integrins β1, β2, α1, α2, α5, and the nonintegrin fibronectin receptor Agp110 in vivo. Cultured rat hepatocytes display integrins α1, α3, α5, β1, and α6μ1, and their expression is modulated by the culture conditions.

### Exemplary Inducible Tissue Constructs

In some embodiments, the disclosure provides an engineered tissue construct comprising a population of hepatocytes and a population of stromal cells, wherein the population of stromal cells is engineered to express an inducible cell death-inducing polypeptide. In some embodiments, the disclosure provides an engineered tissue construct comprising a population of hepatocytes and a population of stromal cells, wherein the population of stromal cells is engineered to express an inducible caspase9 polypeptide.

In some embodiments, the disclosure provides an engineered tissue construct comprising a population of hepatocytes and a population of fibroblasts, wherein the population of fibroblasts is engineered to express an inducible cell death-inducing polypeptide. In some embodiments, the disclosure provides an engineered tissue construct comprising a population of hepatocytes and a population of fibroblasts, wherein the population of fibroblasts is engineered to express an inducible caspase9 polypeptide.

In some embodiments, the disclosure provides an engineered tissue construct comprising a population of hepatocytes, a population of stromal cells and a population of vascular cells, wherein the population of stromal cells is engineered to express an inducible cell death-inducing polypeptide. In some embodiments, the disclosure provides an engineered tissue construct comprising a population of hepatocytes, a population of stromal cells, and a population of vascular cells, wherein the population of stromal cells is engineered to express an inducible caspase9 polypeptide.

In some embodiments, the disclosure provides an engineered tissue construct comprising a population of hepatocytes, a population of fibroblasts and a population of endothelial cells, wherein the population of fibroblasts is engineered to express an inducible cell death-inducing polypeptide. In some embodiments, the disclosure provides an engineered tissue construct comprising a population of hepatocytes, a population of fibroblasts and a population of endothelial cells, wherein the population of fibroblasts is engineered to express an inducible caspase9 polypeptide.

In some embodiments, the disclosure provides an engineered tissue construct comprising a population of hepatocytes, a population of fibroblasts and a population of endothelial cells, wherein the population of fibroblasts is engineered to express a caspase9 unimer operably linked to a dimerization domain.

In some embodiments, the disclosure provides an engineered tissue construct comprising:

(i) a biocompatible scaffold; and

(ii) a population of cells comprising a population of hepatocytes, a population of fibroblasts and a population of endothelial cells, wherein the population of fibroblasts is engineered to express a caspase9 unimer operably linked to a dimerization domain, wherein the population of cells are encapsulated in the biocompatible scaffold.

In some embodiments, the disclosure provides an engineered tissue construct comprising:

(i) a cell aggregate comprising a population of hepatocytes and a population of fibroblasts engineered to express a caspase9 unimer operably linked to a dimerization domain, and

(ii) a pre-templated vessel comprising a population of endothelial cells.

In some embodiments, the disclosure provides an engineered tissue construct comprising:

(i) a cell aggregate comprising a population of hepatocytes and a population of fibroblasts engineered to express a caspase9 unimer operably linked to a dimerization domain,

(ii) a pre-templated vessel comprising a population of endothelial cells; and

(iii) a biocompatible scaffold,

wherein the cell aggregate and the pre-templated vessels are cultured in the biocompatible scaffold.

In some embodiments, the disclosure provides an engineered tissue construct comprising a population of hepatocyte precursor cells and a population of stromal cells, wherein the population of hepatocyte precursor cells is engineered to express an inducible differentiation factor, and wherein the population of stromal cells is engineered to express an inducible cell death-inducing polypeptide.

In some embodiments, the disclosure provides an engineered tissue construct comprising a population of hepatocytes and a population of stromal cells, wherein at least one of the cell populations is engineered to express a polypeptide associated with tissue expansion or organogenesis as described herein.

### Methods of Assembling Tissue Constructs

In some embodiments, an engineered tissue construct is generated by seeding at least one population of cells on a substrate. In some embodiments, an engineered tissue construct is generated by seeding at least one population of cells on a template.

In some embodiments, an engineered tissue construct is generated by first forming cell clusters or cell aggregates of at least one cell population. In some embodiments, a cell cluster or cell aggregate comprises at least one cell population comprising a genetic construct described herein. In some embodiments, a cell cluster or cell aggregate comprises more than one cell population. In some embodiments, a cell cluster or cell aggregate comprises a population of parenchymal cells and a population of non-parenchymal cells. For example, in some embodiments, a cell cluster or cell aggregate comprises a population of hepatocytes and a population of stromal cells (e.g., fibroblasts). In some embodiments, a cell cluster or cell aggregate comprises a population of parenchymal cells and a population of non-parenchymal cells comprising a genetic construct described herein. In some embodiments, a cell cluster or cell aggregate comprises a population of hepatocytes and a population of stromal cells comprising a genetic construct comprising a polynucleotide encoding a cell death-inducing polypeptide. In some embodiments, a cell cluster or cell aggregate is formed and then culture on a substrate or scaffold to form an engineered tissue construct.

In some embodiments, an engineered tissue construct comprises pre-templated vessels or self-assembled vessels. Such structures promote rapid formation of vessels that are spatially delineated, providing novel approaches to vascularizing engineered tissues, treating ischemic diseases, and promoting tissue healing and integration. Implantation of pre-templated vessels into a subject can lead to engraftment, remodeling of the local microenvironment, anastomosis, and formation of stable capillaries within an implanted scaffold that directs blood vessels and blood flow. By employing pre-templated vessels generated in vitro, the subsequent formation of blood vessels in vivo is able to be spatially controlled.

The pre-organization of cells into patterned networks (i.e., cords or cylinders) provides a means to support rapid invasion and integration of host vasculature into the device to generate perfused, functional blood vessels by providing a pre-specified architecture as a template in which the new blood vessels mirror the diameter and architecture of the pre-templated vessels. The architecture of the networks of cells engineered in vitro during the assembly of the patterned biomaterial defines the in vivo architecture (vessel diameters and network topology) of the blood vessel network that forms after implantation. Because these patterned networks act as “blood vessel highways” for the invading host tissue, their organization (patterned orientation, size, density, connectivity) can be engineered to rationally impact the rate and extent of host cell integration, and thus be used as a means to direct revascularization from a well perfused site to reach into and support ischemic tissues. In certain embodiments, the cells and matrix originally in the patterned biomaterial can be partially or entirely replaced by host cells and tissue, with the architecture of the patterned biomaterial being templated and preserved by the new host tissue.

In some embodiments, an engineered tissue construct is generated by seeding at least one population of cells in a three-dimensional (3D) template. In some embodiments, organizing cells and material into spatial arrangements, such as pre-templated vessels and/or cell clusters or islands, is accomplished by physically constraining the placement of cells/material by the use of wells or grooves, or injecting cells into microfluidic channels or oriented void spaces/pores. In some embodiments, pre-templated vascular cells, when incorporated into a tissue construct described herein, provide an architecture for vascular expansion and development in the construct by providing a template for capillary formation. In some embodiments, vascular cells (e.g., endothelial cells) are used to form vessel-like structures. In some embodiments, pre-templated vessels are generated by using pre-patterned biomaterials such as channels in a polydimethylsiloxane (PDMS) substrate and encapsulated in a biocompatible hydrogel scaffold.

In some embodiments, the cells are organized by physically positioning cells with electric fields, magnetic tweezers, optical tweezers, ultrasound waves, pressure waves, or micromanipulators. In some embodiments, cells are organized by patterning the attachment of cells into specific arrangements by seeding them onto fibers. In some embodiments, cells are organized by de novo fabrication such as by layer-by-layer or 3D printing.

In some embodiments, the 3D template is generated by molding, templating, photolithography, printing, deposition, sacrificial molding, stereolithography, or a combination thereof.

In some embodiments, the 3D template is generated using naturally-derived and/or synthetic scaffolding.

In some embodiments, the naturally-derived and/or synthetic scaffolding, is selected from, but is not limited to, fibrin, fibrinogen, fibronectin, collagen, polyorthoester, polyvinyl alcohol, polyamide, polycarbonate, carbohydrates, agarose, alginate, poly(ethylene) glycol, polylactic acid, polyglycolic acid, polycaprolactone, polyvinyl pyrrolidone, a marine adhesive protein, cyanoacrylate, polymeric hydrogel, analogs, or a combination thereof.

In some embodiments, the engineered tissue construct is formed by adding cells directly into or onto an extracellular matrix scaffold, in the absence of collagen. For example, in some embodiments, pre-templated vessels are formed by seeding cells without collagen into pre-existing hollow channels of a 3D template and encapsulating the cells into a biocompatible scaffold.

In some embodiments, the tissue construct does not contain the naturally-derived and/or synthetic scaffolding material. In some embodiments, the tissue construct of the present disclosure is formed in the absence of biocompatible scaffolding.

In some embodiments, the tissue construct contains two or more cell types. In some embodiments, the two or more cell types are co-introduced or sequentially introduced in the engineered tissue construct. For example, in some embodiments, the two or more cell types are introduced in the same spatial position, similar spatial positions, or different spatial positions, relative to each other. In some embodiments, the two or more cell types are introduced into or onto different areas of the engineered tissue construct. For example, pre-templated vessels and/or cell clusters are embedded in a naturally-derived and/or synthetic scaffolding, e.g., collagen, which is further encapsulated in a biocompatible scaffold that is seeded with a distinct cell type.

In some embodiments, the 3D template is naturally-derived and/or synthetic material. For example, in some embodiments, the template is composed of silicone or PDMS. In some embodiments, the scaffold includes a physical solid support such as silicone rubber, plastics, glass, hydroxyapatite, poly-lactic acid, poly-glycolic acid, or other materials. In some embodiments, the template contains one or more channels. In some embodiments, the template contains at least 1, 2, 3, 4, 5, 6, 7, 8, 9, 10, 15, 20, 30, or 40 channels. In some embodiments, the one or more channels are arranged in parallel formation. In some embodiments, the one or more channels are arranged in a non-parallel formation. In some embodiments, the one or more channels are organized with specific branch patterns such as rectilinear grids, bifurcated trees, in 2D or 3D organizations. In some embodiments, the channels are spaced apart by less than about 1 μm, greater than about 1 μm, 2, 4, 5, 8, 10, 15, 20, 25, 30, 40, 50, 80, 100, 150, 200, 250, 300, 500, 700, or 900 μm. In some embodiments, the width of each line, groove and/or structure is less than about 1 μm, greater than about 1 μm, 2, 4, 5, 8, 10, 15, 20, 25, 30, 40, 50, 80, 100, 150, 200, 250, 300, 500, 700, 900 μm, 1 mm, 2 mm, 5 mm, 10 mm, or 20 mm.

In some embodiments, the template contains one or more wells and/or grooves to form one or more cell clusters or islands. In some embodiments, the template contains at least 1, 2, 3, 4, 5, 6, 7, 8, 9, 10, 15, 20, 30, or 40 wells. In some embodiments, the one or more wells are organized with certain spacings of less than about 1 μm, greater than about 1 μm, 2, 4, 5, 8, 10, 15, 20, 25, 30, 40, 50, 80, 100, 150, 200, 250, 300, 500, 700, 900 μm, 1 mm, 2 mm, 5 mm, 10 mm, or 20 mm.

In some embodiments, the biocompatible scaffold functions as the 3D template. In some embodiments, the tissue construct is formed by at least partially encasing the 3D template in a biocompatible scaffold. The 3D template is then removed to create channels, wells and/or grooves in the scaffold. Cells can then be added to the newly created channels, wells and/or grooves of the scaffold to form vessel-like structures and/or clusters or islands of cells. In some embodiments, the 3D template is a carbohydrate lattice that dissolves following incubation in cell media to form empty channels, wells, and/or grooves in the scaffold.

In some embodiments, the tissue construct of the present disclosure is fabricated through the use of a custom 3D printer technology to extrude lattices of carbohydrate glass filaments with predefined diameters, spacings and orientations. In some embodiments, soluble (clinical-grade, sterile) fibrinogen and thrombin are then combined and poured over the lattice. After the solution has polymerized into insoluble fibrin, the carbohydrate filaments are dissolved, leaving behind channels within the fibrin. The channels can then be filled with a suspension of cells, such as endothelial and perivascular cells, in a naturally-derived or synthetic scaffolding (e.g., soluble type I collagen) that subsequently is polymerized to trap the cells within the channels to form pre-templated vessels.

In certain embodiments, cells are encapsulated at a concentration or density of about 0.1×10⁶/mL to about 100×10⁶/mL, or about 0.1×10⁶/mL to about 20×10⁶/mL preferably about 0.5×10⁶/mL, 1, 2, 5, 10 or 15×10⁶/mL. In certain embodiments, non-parenchymal cells of a non-parenchymal cell population cell type are encapsulated at a ratio (as compared to parenchymal cells) of about 0.1:1, 0.5:1, 1:1, 1.5:1, 2:1, 3:1, 5:1 or 10:1. In some embodiments, the above values or ranges are at the time of encapsulation. In some embodiments, the above values or ranges are at a time following encapsulation or implantation, e.g., at about 1, 2, 5, 12, 24, 36, 48, 72, 96 or more hours after encapsulation or implantation, i.e., the cells, e.g., the parenchymal cells and/or one or more non-parenchymal cell populations are encapsulated at a lower concentration or density and proliferate to achieve the indicated concentration or density after a certain time in culture or in vivo.

In some embodiments, cells present in a heterotypic cell suspension are seeded in a ratio of about 50:1, 20:1, 10:1, 5:1, 2:1, or 1:1, but these ratios can vary depending on the type of cells involved. One of ordinary skill in the art, with the benefit of this disclosure, will be able to determine the appropriate ratio of cell types in a heterotypic suspension to achieve the objectives of the present disclosure.

In some embodiments, the ratio of support cells to other cell types present in the cell suspension is from about 1:1000, about 1:100, about 50:1, about 30:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:30, about 1:50, about 1:100 or about 1:1000. In some embodiments, the ratio of endothelial cells to other cell types is from about 50:1 to about 1:3.

In some embodiments, the support cells are present in the cell suspension at a volume percentage from about 1%, 5%, 10%, 15%, 20%, 25%, 30%, 33%, 40%, 50%, 66%, 75%, 80%, 90%, 95%, 99%, or 100%. In some embodiments, the endothelial cells are present in the cell suspension at a volume percentage from about 30%.

In some embodiments, the width and/or diameter of the one or more cell clusters of the present disclosure is less than about 1 μm, greater than about 1 μm, 2 μm, 4 μm, 5 μm, 8 μm, 10 μm, 15 μm, 20 μm, 25 μm, 30 μm, 40 μm, 50 μm, 80 μm, 100 μm, 150 μm, 200 μm, 250 μm, 300 μm, 500 μm, 700 μm, 900 μm, 1 mm, 2 mm, 5 mm, 10 mm, or 20 mm or a combination thereof.

In some embodiments, the spacing between adjacent cell clusters is less than about 1 μm, greater than about 1 μm, 2 μm, 4 μm, 5 μm, 8 μm, 10 μm, 15 μm, μm, 25 μm, 30 μm, 40 μm, 50 μm, 80 μm, 100 μm, 150 μm, 200 μm, 250 μm, 300 μm, 500 μm, 700 μm, 900 μm, 1 mm, 2 mm, 5 mm, 10 mm, or 20 mm or a combination thereof.

In some embodiments, the number of cell clusters and/or islands contained within the tissue construct vary. In some embodiments, the engineered tissue construct includes at least 1, 2, 3, 4, 5, 10, 15, 20, 25, 30, 35, 50, 100, 150, 200, 300, 400, 500, 1000, 10,000, 100,000, or 1,000,000 organized cell clusters and/or islands.

In some embodiments, dielectrophoresis (DEP) is used for patterning of cells in relatively homogeneous slabs of hydrogel or in conjunction with the photopolymerization method. The methods allow for the formation of 3D scaffolds from hundreds of microns to tens of centimeters in length and width, and tens of microns to hundreds of microns in height. A resolution of up to 100 microns in the photopolymerization method and possible single cell resolution (10 micron) in the DEP method is achievable. Photopolymerization apparatus, DEP apparatus, and other methods to produce the 3-dimensional co-cultures of the invention are described in U.S. patent application Ser. No. 11/035,394.

Methods to assemble 3D devices and seeding of cells are known to those of skill in the art. For example, one method includes constructing molds for a 2-channel microfluidic device using stereolithography (Proto Labs).

### Methods for Eliminating Cells in a Tissue Construct

In some embodiments, the present disclosure provides methods for eliminating at least one cell population from a tissue construct.

In some embodiments, a method for eliminating a cell population from a tissue construct comprises:

(a) introducing a genetic construct encoding an inducible cell death-inducing polypeptide into a cell population;

(b) seeding the cell population of (a) onto a substrate or scaffold to form a tissue construct;

(c) contacting the tissue construct with a stimulus to activate the inducible cell death-inducing polypeptide, such that the cell population is eliminated from the tissue construct.

In some embodiments, a method for eliminating a cell population from a tissue construct comprises:

(a) introducing a genetic construct encoding an inducible caspase9 unimer operably linked to a dimerization domain into a cell population;

(b) seeding the cell population of (a) onto a substrate or scaffold to form a tissue construct;

(c) contacting the tissue construct with CID to activate caspase9, such that apoptosis is induced and the cell population is eliminated from the tissue construct.

In some embodiments, a method for eliminating at least one cell population from a tissue construct comprises:

(a) introducing a genetic construct encoding an inducible cell death-inducing polypeptide into a first cell population;

(b) culturing the cell population of (a) with a second cell population under conditions sufficient to form a tissue construct;

(c) contacting the tissue construct with a stimulus to activate the inducible cell death-inducing polypeptide, such that the first cell population is eliminated from the tissue construct.

In some embodiments, elimination of the cell population occurs in vitro. In some embodiments, the tissue construct is contacted with the stimulus in vitro. In some embodiments, elimination of the cell population occurs in vivo. In some embodiments, the tissue construct is implanted in a subject and the after implantation the subject is administered the stimulus.

In some embodiments, at least one population of cells within a tissue construct comprises an inactive constitutively expressed cell death-inducing polypeptide (e.g., a caspase9 unimer), wherein the cell death-inducing polypeptide is activated upon dimerization. In some embodiments, dimerization is induced by a chemical inducer of dimerization (CID) as described herein. In some embodiments, the engineered tissue construct is contacted with 5 nM, 50 nM, or 500 nM of any CID described herein. In some embodiments, wherein the tissue construct is implanted in vivo, 0.5-10 mg/kg of CID is administered to induce cell-death. In some embodiments, the engineered tissue construct is contacted for 15 minutes, 30 minutes, 1 hour, 2 hours, 3 hours, 4 hours, 5 hours, 6 hours, 24 hours, 2 days, 3 days, 4 days, 5 days, 6 days, 7 days, 8 days, 9 days, 10 days, 11 days. 12 days, 13 days, 14 days, 15 days, 16 days, 17 days, 18 days, 19 days, 20 days, 21 days, 22 days, 23 days, 24 days, or 25 days with any CID described herein.

In some embodiments, at least one population of cells is engineered to comprise an inactive constitutively expressed caspase domain. In some embodiments, homodimerization of caspase domains in the presence of CID results in caspase activation, which is 2, 5, 10, 50, 100, 1,000 or 10,000-fold higher than the caspase activity which occurs in the absence of CID.

In some embodiments, a method for eliminating at least one cell population from a tissue construct comprises:

(a) introducing a genetic construct encoding an inducible caspase9 unimer operably linked to a dimerization domain into a first cell population;

(b) culturing the cell population of (a) with a second cell population under conditions sufficient to form a tissue construct;

(c) contacting the tissue construct with CID to activate caspase9, such that apoptosis is induced and the first cell population is eliminated from the tissue construct.

In some embodiments, a method for eliminating stromal cells from a tissue construct comprises:

(a) introducing a genetic construct encoding an inducible cell death-inducing polypeptide into a population of stromal cells;

(b) culturing the cells of (a) with at least one additional cell population under conditions sufficient to form a tissue construct;

(c) contacting the tissue construct with a stimulus to activate the inducible cell death-inducing polypeptide, such that the stromal cells are eliminated from the tissue construct.

In some embodiments, a method for eliminating stromal cells from a tissue construct comprises:

(a) introducing a genetic construct encoding an inducible caspase9 unimer operably linked to a dimerization domain into a population of stromal cells;

(b) culturing the cell population of (a) with at least one additional cell population under conditions sufficient to form a tissue construct;

(c) contacting the tissue construct with CID to activate caspase9, such that apoptosis is induced and the stromal cells are eliminated from the tissue construct.

In some embodiments, a method for eliminating stromal cells from a liver tissue construct comprises:

(a) introducing a genetic construct encoding an inducible cell death-inducing polypeptide into a population of stromal cells;

(b) culturing the cell population of (a) with a population of hepatocytes under conditions sufficient to form a liver tissue construct;

(c) contacting the liver tissue construct with a stimulus to activate the inducible cell death-inducing polypeptide, such that the stromal cells are eliminated from the tissue construct.

In some embodiments, a method for eliminating stromal cells from a liver tissue construct comprises:

(a) introducing a genetic construct encoding an inducible caspase9 unimer operably linked to a dimerization domain into a population of stromal cells;

(b) culturing the cell population of (a) with a population of hepatocytes under conditions sufficient to form a liver tissue construct;

(c) contacting the liver tissue construct with CID to activate caspase9, such that apoptosis is induced and the stromal cells are eliminated from the tissue construct.

In some embodiments, a method for eliminating stromal cells from a liver tissue construct comprises:

(a) introducing a genetic construct encoding an inducible cell death-inducing polypeptide into a population of stromal cells;

(b) culturing the cell population of (a) with a population of hepatocytes and a population of endothelial cells under conditions sufficient to form a liver tissue construct;

(c) contacting the liver tissue construct with a stimulus to activate the inducible cell death-inducing polypeptide, such that the stromal cells are eliminated from the tissue construct.

In some embodiments, a method for eliminating stromal cells from a liver tissue construct comprises:

(a) introducing a genetic construct encoding an inducible caspase9 unimer operably linked to a dimerization domain into a population of stromal cells;

(b) culturing the cell population of (a) with a population of hepatocytes and a population of endothelial cells under conditions sufficient to form a liver tissue construct;

(c) contacting the liver tissue construct with CID to activate caspase9, such that apoptosis is induced and the stromal cells are eliminated from the tissue construct.

In some embodiments, a method for eliminating endothelial cells from a tissue construct comprises:

(a) introducing a genetic construct encoding an inducible cell death-inducing polypeptide into a population of endothelial cells;

(b) culturing the cells of (a) with at least one additional cell population under conditions sufficient to form a tissue construct;

(c) contacting the tissue construct with a stimulus to activate the inducible cell death-inducing polypeptide, such that the endothelial cells are eliminated from the tissue construct.

In some embodiments, a method for eliminating endothelial cells from a tissue construct comprises:

(a) introducing a genetic construct encoding an inducible caspase9 unimer operably linked to a dimerization domain into a population of endothelial cells;

(b) culturing the cell population of (a) with at least one additional cell population under conditions sufficient to form a tissue construct;

(c) contacting the tissue construct with CID to activate caspase9, such that apoptosis is induced and the endothelial cells are eliminated from the tissue construct.

In some embodiments, a method for eliminating endothelial cells from a liver tissue construct comprises:

(a) introducing a genetic construct encoding an inducible cell death-inducing polypeptide into a population of endothelial cells;

(b) culturing the cell population of (a) with a population of hepatocytes under conditions sufficient to form a liver tissue construct;

(c) contacting the liver tissue construct with a stimulus to activate the inducible cell death-inducing polypeptide, such that the endothelial cells are eliminated from the tissue construct.

In some embodiments, a method for eliminating endothelial cells from a liver tissue construct comprises:

(a) introducing a genetic construct encoding an inducible caspase9 unimer operably linked to a dimerization domain into a population of endothelial cells;

(b) culturing the cell population of (a) with a population of hepatocytes under conditions sufficient to form a liver tissue construct;

(c) contacting the liver tissue construct with CID to activate caspase9, such that apoptosis is induced and the endothelial cells are eliminated from the tissue construct.

In some embodiments, a method for eliminating endothelial cells from a liver tissue construct comprises:

(a) introducing a genetic construct encoding an inducible cell death-inducing polypeptide into a population of endothelial cells;

(b) culturing the cell population of (a) with a population of hepatocytes and a population of stromal cells under conditions sufficient to form a liver tissue construct;

(c) contacting the liver tissue construct with a stimulus to activate the inducible cell death-inducing polypeptide, such that the endothelial cells are eliminated from the tissue construct.

In some embodiments, a method for eliminating endothelial cells from a liver tissue construct comprises:

(a) introducing a genetic construct encoding an inducible caspase9 unimer operably linked to a dimerization domain into a population of endothelial cells;

(b) culturing the cell population of (a) with a population of hepatocytes and a population of stromal cells under conditions sufficient to form a liver tissue construct;

(c) contacting the liver tissue construct with CID to activate caspase9, such that apoptosis is induced and the endothelial cells are eliminated from the tissue construct.

In some embodiments, a method for eliminating stromal and endothelial cells from a tissue construct comprises:

(a) introducing a genetic construct encoding an inducible cell death-inducing polypeptide into a population of endothelial cells and a population of stromal cells;

(b) culturing the cell populations of (a) with at least one additional cell population under conditions sufficient to form a tissue construct;

(c) contacting the tissue construct with a stimulus to activate the inducible cell death-inducing polypeptide, such that the endothelial and stromal cells are eliminated from the tissue construct.

In some embodiments, a method for eliminating endothelial and stromal cells from a tissue construct comprises:

(a) introducing a genetic construct encoding an inducible caspase9 unimer operably linked to a dimerization domain into a population of endothelial cells and a population of stromal cells;

(b) culturing the cell populations of (a) with at least one additional cell population under conditions sufficient to form a tissue construct;

(c) contacting the tissue construct with CID to activate caspase9, such that apoptosis is induced and the endothelial and stromal cells are eliminated from the tissue construct.

In some embodiments, a method for eliminating endothelial and stromal cells from a liver tissue construct comprises:

(a) introducing a genetic construct encoding an inducible cell death-inducing polypeptide into a population of endothelial cells and a population of stromal cells;

(b) culturing the cell population of (a) with a population of hepatocytes under conditions sufficient to form a liver tissue construct;

(c) contacting the liver tissue construct with a stimulus to activate the inducible cell death-inducing polypeptide, such that the endothelial and stromal cells are eliminated from the tissue construct.

In some embodiments, a method for eliminating endothelial and stromal cells from a liver tissue construct comprises:

(a) introducing a genetic construct encoding an inducible caspase9 unimer operably linked to a dimerization domain into a population of endothelial cells and a population of stromal cells;

(b) culturing the cell population of (a) with a population of hepatocytes under conditions sufficient to form a liver tissue construct;

(c) contacting the liver tissue construct with CID to activate caspase9, such that apoptosis is induced and the endothelial and stromal cells are eliminated from the tissue construct.

### Methods for Tissue Expansion and Organogenesis

In some embodiments, the present disclosure provides methods for inducing tissue organogenesis of an engineered tissue construct. In some embodiments, the present disclosure provides methods for inducing expansion of an engineered tissue construct.

In some embodiments, a method for inducing expansion of an engineered tissue construct comprises:

(a) introducing a genetic construct into at least one cell population, wherein the genetic construct comprises a polynucleotide encoding a polypeptide of interest comprising an inducible element, wherein the polypeptide is activated upon interaction of the inducible element with a biological molecule or small molecule;

(b) culturing the cell population of (a) onto a substrate to form a tissue construct;

(c) contacting the tissue construct with the biological molecule or small molecule, such that expression of the polypeptide induces expansion of the engineered tissue construct.

In some embodiments, a method for inducing expansion of an engineered tissue construct comprises:

(a) introducing a genetic construct into at least one cell population, wherein the genetic construct comprises a polynucleotide encoding a polypeptide of interest comprising an inducible element, wherein the polypeptide is activated upon interaction of the inducible element with a biological molecule or small molecule;

(b) culturing the cell population of (a) with at least one other cell population onto a substrate to form a tissue construct;

(c) contacting the tissue construct with the biological molecule or small molecule, such that expression of the polypeptide induces expansion of the engineered tissue construct.

In some embodiments, a method for inducing expansion of an engineered tissue construct comprises:

(a) introducing a genetic construct into at least one population of cells, wherein the genetic construct comprises a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;

(b) culturing the cell population of (a) onto a substrate to form a tissue construct;

(c) contacting the tissue construct with a stimulus of the inducible promoter, such that expression of the polypeptide induces expansion of the engineered tissue construct.

In some embodiments, a method for inducing expansion of an engineered tissue construct comprises:

(a) introducing a genetic construct into at least one population of cells, wherein the genetic construct comprises a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;

(b) culturing the cell population of (a) with at least one other cell population onto a substrate to form a tissue construct;

(c) contacting the tissue construct with a stimulus of the inducible promoter, such that expression of the polypeptide induces expansion of the engineered tissue construct.

In some embodiments, a method for inducing tissue organogenesis of an engineered tissue construct comprises:

(a) introducing a genetic construct into at least one cell population, wherein the genetic construct comprises a polynucleotide encoding a differentiation factor comprising an inducible element, wherein the differentiation factor is activated upon interaction of the inducible element with a biological molecule or small molecule;

(b) culturing the cell population of (a) onto a substrate to form a tissue construct;

(c) contacting the tissue construct with the biological molecule or small molecule, such that expression of the differentiation factor induces tissue organogenesis of the engineered tissue construct.

In some embodiments, a method for inducing tissue organogenesis of an engineered tissue construct comprises:

(a) introducing a genetic construct into at least one cell population, wherein the genetic construct comprises a polynucleotide encoding a differentiation factor comprising an inducible element, wherein the differentiation factor is activated upon interaction of the inducible element with a biological molecule or small molecule;

(b) culturing the cell population of (a) with at least one other cell population onto a substrate to form a tissue construct;

(c) contacting the tissue construct with the biological molecule or small molecule, such that expression of the differentiation factor induces tissue organogenesis of the engineered tissue construct.

In some embodiments, a method for inducing tissue organogenesis of an engineered tissue construct comprises:

(a) introducing a genetic construct into at least one population of cells, wherein the genetic construct comprises a polynucleotide comprising an inducible promoter operably linked to a nucleotide sequence encoding a differentiation factor, wherein expression of differentiation factor is controlled by the inducible promoter;

(b) culturing the cell population of (a) onto a substrate to form a tissue construct;

(c) contacting the tissue construct with a stimulus of the inducible promoter, such that expression of the differentiation factor induces tissue organogenesis of the engineered tissue construct.

In some embodiments, a method for inducing expansion of an engineered tissue construct comprises:

(a) introducing a genetic construct into at least one population of cells, wherein the genetic construct comprises a polynucleotide comprising an inducible promoter operably linked to a nucleotide sequence encoding a differentiation factor, wherein expression of the polypeptide or nucleic acid molecule is controlled by the inducible promoter;

(b) culturing the cell population of (a) with at least one other cell population onto a substrate to form a tissue construct;

(c) contacting the tissue construct with a stimulus of the inducible promoter, such that expression of the differentiation factor induces tissue organogenesis of the engineered tissue construct.

In some embodiments, a method for inducing cell proliferation in an engineered tissue construct comprises:

(a) introducing a genetic construct into at least one cell population, wherein the genetic construct comprises a polynucleotide encoding a polypeptide of interest comprising an inducible element, wherein the polypeptide is activated upon interaction of the inducible element with a biological molecule or small molecule;

(b) culturing the cell population of (a) onto a substrate to form a tissue construct;

(c) contacting the tissue construct with the biological molecule or small molecule, such that expression of the polypeptide induces cell proliferation within the engineered tissue construct.

In some embodiments, a method for inducing cell proliferation in an engineered tissue construct comprises:

(a) introducing a genetic construct into at least one cell population, wherein the genetic construct comprises a polynucleotide encoding a polypeptide of interest comprising an inducible element, wherein the polypeptide is activated upon interaction of the inducible element with a biological molecule or small molecule;

(b) culturing the cell population of (a) with at least one other cell population onto a substrate to form a tissue construct;

(c) contacting the tissue construct with the biological molecule or small molecule, such that expression of the polypeptide induces cell proliferation within the engineered tissue construct.

In some embodiments, a method for inducing cell proliferation of an engineered tissue construct comprises:

(a) introducing a genetic construct into at least one population of cells, wherein the genetic construct comprises a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;

(b) culturing the cell population of (a) onto a substrate to form a tissue construct;

(c) contacting the tissue construct with a stimulus of the inducible promoter, such that expression of the polypeptide or nucleic acid molecule induces cell proliferation within the engineered tissue construct.

In some embodiments, a method for inducing expansion of an engineered tissue construct comprises:

(a) introducing a genetic construct into at least one population of cells, wherein the genetic construct comprises a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;

(b) culturing the cell population of (a) with at least one other cell population onto a substrate to form a tissue construct;

(c) contacting the tissue construct with a stimulus of the inducible promoter, such that expression of the polypeptide or nucleic acid molecule induces cell proliferation within the engineered tissue construct.

In some embodiments, the polypeptide of interest the induces cell proliferation and/or expansion of the engineered tissue construct is Wnt2, EGF, HGF, FGF, VEGF, IL-8, Ang-RSPO, GATA4, IL-6, DLL4, ID-1, PGE2 or CSF1.

In some embodiments, cell proliferation, tissue organogenesis and/or tissue expansion of the engineered tissue construct is induced in vitro. In some embodiments, cell proliferation, tissue organogenesis and/or tissue expansion is induced in vivo after the engineered tissue construct has been implanted in a subject.

### Applications Using the Tissue Constructs

In some embodiments, the engineered tissue construct of the present disclosure is implanted in a subject. Non-limiting examples of non-human subjects include non-human primates, dogs, cats, mice, rats, guinea pigs, rabbits, fowl, pigs, horses, cows, goats, sheep, etc. In some embodiments, the subject is any animal. In some embodiments, the subject is any mammal. In some embodiments, the subject is a human. In some embodiments, the engineered tissue construct is implanted in a subject by placing the engineered tissue construct onto fat pads in the lower abdomen. In some embodiments, an engineered tissue construct is placed on a fat pat located in, but not limited to, parametrial, mesenteric, or ornental spaces. In some embodiments, an engineered tissue construct is placed in the subcutaneous space or intramuscular space. In some embodiments, constructs are implanted by suturing, use of surgical glue, or in situ polymerization.

In some embodiments, cell death of a non-parenchymal cell population in an engineered tissue construct is induced after implantation of the engineered tissue construct into a subject. In some embodiments, cell death of a non-parenchymal cell population in an engineered tissue construct is induced prior to implantation of the engineered tissue construct into a subject. In some embodiments, apoptosis of a non-parenchymal cell population in an engineered tissue construct is induced after implantation of the engineered tissue construct into a subject. In some embodiments, apoptosis of a non-parenchymal cell population in an engineered tissue construct is induced prior to implantation of the engineered tissue construct.

In some embodiments, cell death of a parenchymal cell population in an engineered tissue construct is induced after implantation of the engineered tissue construct into a subject. In some embodiments, cell death of a parenchymal cell population in an engineered tissue construct is induced prior to implantation of the engineered tissue construct into a subject. In some embodiments, apoptosis of a parenchymal cell population in an engineered tissue construct is induced after implantation of the engineered tissue construct into a subject. In some embodiments, apoptosis of a parenchymal cell population in an engineered tissue construct is induced prior to implantation of the engineered tissue construct.

In some embodiments, expansion of an engineered tissue construct is induced after implantation of the engineered tissue construct into a subject. In some embodiments, expansion of an engineered tissue construct is induced prior to implantation of the engineered tissue construct into a subject. In some embodiments, cell proliferation of at least one cell population in an engineered tissue construct is induced after implantation of the engineered tissue construct into a subject. In some embodiments, cell proliferation of at least one cell population in an engineered tissue construct is induced prior to implantation of the engineered tissue construct into a subject. In some embodiments, cell differentiation of at least one cell population in an engineered tissue construct is induced after implantation of the engineered tissue construct into a subject. In some embodiments, cell differentiation of at least one cell population in an engineered tissue construct is induced prior to implantation of the engineered tissue construct into a subject.

In some embodiments, the engineered tissue construct is used to enhance vascularization in ischemic settings, such as, by acting as a conduit to increase blood flow to regions of tissues that are not receiving sufficient blood supply. In some embodiments, the engineered tissue construct is implanted in a region of a subject that requires an increase in blood flow. For example, the engineered tissue construct is implanted in and/or near an ischemic tissue. In some embodiments, the engineered tissue construct is implanted to treat cardiac ischemia. In some embodiments, the engineered tissue construct is implanted to revascularize from healthy coronary circulation or neighboring non-coronary vasculature.

In some embodiments, the engineered tissue construct is used as a “directional microbypass” for revascularizing ischemic myocardium not amenable to traditional therapies. In many patients that suffer from acute myocardial ischemia and in another even larger cohort of patients with untreatable coronary disease, there remain areas of viable heart that are not revascularized. In some embodiments, the engineered tissue construct can potentially revascularize those inaccessible ischemic zones in these patients. The pre-templated vessels of the engineered tissue construct can stimulate and spatially direct revascularization and thus can form a “vascular bridge” from nearby unobstructed coronary vasculature to around and beyond a coronary obstruction leading to micro-perfused distal myocardium to protect cardiomyocytes viability and function.

In some embodiments, the engineered tissue construct of the present disclosure enhances neovascularization as well as influence vascular architecture through two potential mechanisms. In some embodiments, the embedded pre-templated vessels themselves are incorporated into new capillary networks. Furthermore, in some embodiments, embedded pre-templated vessels deposit additional matrix and secrete growth factors into the scaffold thereby providing a microenvironment that more closely mimics that of native tissue. In some embodiments, the engineered tissue construct is capable of enhancing neovascularization by spatially guiding the invading sprouts of an angiogenic capillary network upon implantation, without incorporation into the nascent vessels. In some embodiments, the engineered tissue construct of the present disclosure is used in conjunction with various types of engineered tissue constructs to aid in the vascularization of the engineered tissue construct.

In some embodiments, the engineered tissue constructs of the present disclosure are used in applications in which it would be beneficial to have an engineered material to aid in spatially guiding the direction of host cell and tissue invasion. Such applications include, but are not limited to, nerve regeneration. In some embodiments, the components of the heterotypic cell suspension used to fabricate the pre-templated vessels described herein are modified for a specific application. For example, for nerve regeneration applications, the cell suspension can include neurons, neuronal stem cells, or cells that are associated with supporting neuronal function, or a combination thereof. In some embodiments, the engineered tissue construct is implanted at a site of tissue damage, e.g., neuronal tissue damage.

In some embodiments, the components of the biocompatible scaffold used to fabricate the engineered tissue construct of the present disclosure are modified for a specific application. For example, in some embodiments, for nerve regeneration applications, the biocompatible scaffold includes neurons, and the engineered tissue construct is used at a site of tissue damage, e.g., neuronal tissue damage.

In some embodiments, the engineered tissue construct of the present disclosure allows for maintenance of the viability and proper function of an engineered tissue. For example, in some embodiments, the engineered tissue construct allows for maintenance of the viability and proper function of an engineered liver tissue construct. In some embodiments, the engineered tissue construct of the present disclosure is used to enhance the survival and function of hepatocytes within large engineered liver constructs upon implantation. Effective mass transport between the blood stream and the liver for metabolic needs relies on a precisely-defined microenvironment delineated by the paracrine and juxtacrine signaling between hepatocytes and endothelial cells. As such, the liver serves as an ideal model to study the interaction between organized endothelial networks and cellular function. In addition, the material can also be used to support function of many other engineered tissues including, but not limited to, bone, fat, muscle, heart, and pancreas.

In some embodiments, the engineered tissue constructs of the present disclosure enhance wound healing. In some embodiments, the engineered tissue constructs are used in treatment of chronic wounds, for example, diabetic foot ulcers. In some embodiments, the engineered tissue construct is implanted in a subject to treat peripheral vascular disease, diabetic wounds, and clinical ischemia.

In some embodiments, the engineered tissue construct of the present disclosure enhances repair of various tissues. In some embodiments, the engineered tissue is used to treat conditions or disorders related to, but not limited to, skeletal muscle tissue, skin, fat tissue, bone, cardiac tissue, pancreatic tissue, liver tissue, lung tissue, kidney tissue, intestinal tissue, esophageal tissue, stomach tissue, nerve tissue, spinal tissue, and brain tissue.

In some embodiments, a method of vascularizing a tissue of a subject includes providing a engineered tissue construct comprising organized endothelial-based pre-templated vessels and implanting the engineered tissue construct into a tissue of the subject, wherein the biomaterial promotes increased vascularity and perfusion in the subject.

In some embodiments, an engineered tissue construct of the present disclosure is used to replace aberrant gene expression. In some embodiments, the engineered tissue construct is used to return gene expression levels to normal physiological levels. In some embodiments, the engineered tissue construct increases gene expression by at least 20%, at least 30%, at least 40%, at least 50%, at least 60%, at least 70%, at least 80%, at least 90%, or at least 99% compared to endogenous gene expression in the subject.

Treatment of Metabolic Disorders

In some embodiments, the disclosure provides methods of treating a metabolic disorder with any engineered tissue construct described herein. In some embodiments, an engineered tissue construct described herein is used to treat a metabolic disorder. Metabolic disorders are caused by genetic deficiencies or result from external factors such as food consumption. Metabolic disorders can take form in the liver, pancreas, endocrine glands, or other organs involved in metabolism. Missing or dysfunctional enzymes, nutritional deficiencies, and abnormal chemical reactions are examples of processes which may lead to a metabolic disorder.

In some embodiments, the disclosure provides methods of treating any one or more of urea cycle disorders, clotting disorders, storage disorders, lipid disorders, non-urea metabolic disorders, congenital diseases (e.g. cystic fibrosis, alpha-1 antitrypsin deficiency, primary hyperoxaluria type 1, or non-monogenic diseases (acute-on chronic liver failure) with any tissue construct described herein. In some embodiments, a tissue construct described herein is used to treat any one or more of urea cycle disorders, clotting disorders, storage disorders, lipid disorders, non-urea metabolic disorders, congenital diseases (e.g. cystic fibrosis, alpha-1 antitrypsin deficiency, primary hyperoxaluria type 1, or non-monogenic diseases (acute-on chronic liver failure).

In some embodiments, the disclosure provides methods of treating a metabolic disorder listed in Table 1 with any engineered tissue construct described herein. In some embodiments, the disclosure provides methods of treating a metabolic disorder caused by any mutation listed in Table 1 with any engineered tissue construct described herein. In some embodiments, an engineered tissue construct described herein is used to treat any metabolic disorder listed in Table 1. In some embodiments, an engineered tissue construct described herein is used to treat a metabolic disorder caused by any mutation listed in Table 1.

TABLE 1
Metabolic disorders and their associated genetic mutations

| Category | Disease | Mutation |
|---|---|---|
| Urea cycle disorders | Ornithine transcarbamylase | OTC |
| | deficiency | |
| | Carbamoyl phosphate | CPS1 |
| | synthetase I | |
| | Argininosuccinic aciduria | ASL |
| | Citrullinemia type I | ASS1 |
| | Arginase deficiency | ARG1 |
| Clotting disorders | Hemophilia A | F8 |
| | Hemophilia B | F9 |
| | Factor VII deficiency | F7 |
| | Factor X deficiency | F10 |
| Storage disorders | Hereditary hemochromatosis | TFR2 |
| | Hereditary hemochromatosis | HAMP |
| | Hereditary hemochromatosis | HJV |
| | Hereditary hemochromatosis | HFE |
| | Glycogen storage disease 1a | G6PC |
| | Glycogen storage disease 1b | SLC37A4 |
| | Wilson's disease | ATP7B |
| Lipid disorders | Familial hypercholesterolemia | LDLR |
| | Familial hypercholesterolemia | LDLRAP1 |
| | Familial hypercholesterolemia | PCSK9 |
| | Familial hypercholesterolemia | APOB |
| Non-urea | Hereditary tyrosinemia | FAH |
| metabolic disorders | Crigler-Najjar syndrome | UGT1A1 |
| | Phenylketonuria | PAH |
| Other congenital diseases | Cystic fibrosis | CFTR |
| | alpha-1 antitrypsin deficiency | SERPINA1 |
| | Primary hyperoxaluria type 1 | AGXT |
| Other, non-monogenic | Acute-on-chronic liver failure | N/A |

In some embodiments, any engineered tissue construct described herein comprises at least one cell population engineered to express a gene associated with a metabolic disorder. In some embodiments, any engineered tissue construct described herein comprises at least one cell population engineered to express any one of the genes described in Table 1, and any combination thereof. In some embodiments, a hepatocyte or population of hepatocytes is engineered to express at least one gene associated with a metabolic disorder prior to incorporation into an engineered tissue construct described herein.

In some embodiments, an engineered tissue construct described herein is used for treating acute-on-chronic liver failure, which is brought upon by non-genetic causes such as viral (e.g., hepatitis) or bacterial infection, or insult with toxins (e.g., alcohol).

In some embodiments, the disclosure provides methods for treating urea cycle disorders. In some embodiments, an engineered tissue construct described herein is used to treat a urea cycle disorder. Urea cycle disorders are inherited diseases. When the body breaks down excess amino acids, they are processed into ammonia. Ammonia is then cycled into urea to be released through urine. Urea cycle disorders are caused when an enzyme involved in this process is missing or dysregulated. A block in this process causes a harmful ammonia buildup in the body leading to various disease symptoms.

In some embodiments, the urea cycle disorder is any one of, but not limited to, Ornithine transcarbamylase deficiency, Carbamoyl phosphate synthetase I, Argininosuccinic aciduria, Citrullinemia type I, or Citrullinemia type I. In some embodiments, the urea cycle disorder is Ornithine transcarbamylase deficiency. In some embodiments, the urea cycle disorder is Carbamoyl phosphate synthetase I. In some embodiments, the urea cycle disorder is Argininosuccinic aciduria. In some embodiments, the urea cycle disorder is Citrullinemia type I. In some embodiments, the urea cycle disorder is Arginase deficiency.

In some embodiments, the disclosure provides methods for treating urea cycle disorders associated with any one or more of the genes selected from OTC, CPS1, ASL, ASS1, and ARG1. In some embodiments, the engineered tissue construct comprises a cell population comprising a genetic construct comprising a functional OTC gene. In some embodiments, the engineered tissue construct comprises a cell population comprising a genetic construct comprising a functional CPS1 gene. In some embodiments, the engineered tissue construct comprises a cell population comprising a genetic construct comprising a functional ASL gene. In some embodiments, the engineered tissue construct comprises a cell population comprising a genetic construct comprising a functional ASS1 gene. In some embodiments, the engineered tissue construct comprises a cell population comprising a genetic construct comprising a functional ARG1 gene.

In some embodiments, the disclosure provides methods for reducing ammonia levels in a subject using an engineered tissue construct described herein. In some embodiments, a subject is treated with an engineered tissue construct expressing at least one of the genes selected from OTC, CPS1, ASL, ASS1, and ARG1 to reduce ammonia levels. In some embodiments, the disclosure provides methods for reducing ammonia levels in a subject to normal physiological levels using an engineered tissue construct. In some embodiments, the disclosure provides methods for reducing ammonia levels in a subject to normal physiological levels using an engineered tissue construct expressing at least one of the genes selected from OTC, CPS1, ASL, ASS1, and ARG1. In some embodiments, disease symptoms associated with urea cycle disorders are reduced after treatment with an engineered tissue construct.

In some embodiments, the engineered tissue construct comprises a population of hepatocytes that expresses any one or more of the genes selected from OTC, CPS1, ASL, ASS1, and ARG1.

In some embodiments, the engineered tissue construct reduces the symptoms associated with metabolic disease. In some embodiments, where a metabolic disease is caused by loss of a gene, the engineered tissue construct increases said gene expression by at least 20%, at least 30%, at least 40%, at least 50%, at least 60%, at least 70%, at least 80%, at least 90%, or at least 99%.

### Definitions

Terms used in the claims and specification are defined as set forth below unless otherwise specified.

As used herein and in the appended claims, the singular forms “a,” “and,” and “the” include plural referents unless the context clearly dictates otherwise. Thus, for example, reference to “a cellular aggregate” includes a plurality of such cellular aggregates and reference to “the cell” includes reference to one or more cells known to those skilled in the art, and so forth.

The term “apoptosis” refers to the process of programmed cell death. The intracellular machinery responsible for apoptosis depends on caspases. Once activated, caspases cleave other caspases and key proteins in the cell that leads to blebbing, cell shrinkage, nuclear fragmentation, chromatin condensation, chromosomal DNA fragmentation, and global mRNA decay.

The term “biocompatible scaffold” as used herein refers to a support which may have an affinity to cells and be made of a material having a “cell adhesive surface.” In some embodiments, cells are attached to a biocompatible scaffold. In some embodiments, cells are encapsulated or entrapped in a biocompatible scaffold. In some embodiments, cells are associated with a biocompatible scaffold.

The term “biological molecule” refers to a substance produced by a cell or a living organism. There are for major classes of biological macromolecules: carbohydrates, lipids, proteins, and nucleic acids.

The term “caspase” refers to a protein within a family of proteases that have a cysteine at their active site and cleave their target proteins as specific aspartic acids. Caspases are synthesized in the cell as inactive precursors, also called procaspases, which are usually activated by cleavage at aspartic acids by other caspases. Initiator procaspases are brought together by adaptor proteins to form a complex that causes the caspases to cleave each other and trigger mutual activation.

The term “cell death” refers to the event of irreversible degeneration of vital cellular functions culminating in the loss of cellular integrity (e.g., permanent plasma membrane permeabilization). Many types of cell death exist including, but not limited to, necroptosis, pyroptosis and anoikis.

The term “cell death-inducing polypeptide” refers to a polypeptide or active fragment thereof capable of inducing cell death. In some embodiments, the cell death-inducing polypeptide is constitutively inactive and requires a stimulus (endogenous or exogenous) to induce cell death. In some embodiments, the cell death-inducing polypeptide is constitutively active.

As used herein, the term “co-culture” refers to a collection of cells cultured in a manner such that more than one population of cells are in association with each other. Co-cultures can be made such that cells exhibit heterotypic interactions (i.e., interaction between cells of populations of different cell types), homotypic interactions (i.e., interaction between cells of the same cell types) or co-cultured to exhibit a specific and/or controlled combination of heterotypic and homotypic interactions between cells.

As used herein, “contacting” refers to either placing a cell or population of cells on a substrate, or placing a molecule of interest (e.g., genetic construct, cell death-inducing polypeptide) in a cell or population of cells, or an engineered tissue construct.

As used herein, the terms “cross-linked” and “linked” are used interchangeably and refer to an attachment of two chains of polymer molecules by bridges, composed of either an element, a group, or a compound, that join certain atoms of the chains by chemical bonds. Cross-linking can be effected naturally and artificially. Internal cross-linking between two sites on a single polymer molecular is also possible.

The terms “cross-linker” or “cross-linking agent”, as used herein, refers to the element, group, or compound that effects cross-linking between polymer chains.

As used herein, the term “ectopic” means occurring in an abnormal position or place. Accordingly, “implantation at an ectopic site” means implantation at an abnormal site or at a site displaced from the normal site. Exemplary ectopic sites of implantation include, but are not limited to the intraperitoneal space and ventral subcutaneous space. Ectopic sites of implantation can also be within an organ, i.e., an organ different than that of the source cells of the construct being implanted (e.g., implanting a human liver construct into the spleen of an animal). Ectopic sites of implantation can also include other body cavities capable of housing a construct described herein. In some embodiments, ectopic sites include, for example, lymph nodes. At least one unexpected feature of the constructs described herein is that constructs implanted at ectopic sites in animals survive, expand, and maintain differentiated function for significant periods of time. This is in contrast to the art-recognized belief that implantation at an orthotopic site (i.e., occurring in a normal position or place) is required to provide trophic factors necessary to support viability (e.g., trophic factors from the gut necessary to support viability in transplanted hepatocyte systems). The term “ectopic” and “heterotopic” can be used interchangeably herein.

As used herein, the term “encapsulation” refers to the confinement of a cell or population of cells within a material, in particular, within a biocompatible scaffold or hydrogel. The term “co-encapsulation” refers to encapsulation of more than one cell or cell type or population or populations of cells within the material, e.g., the polymeric scaffold or hydrogel.

As used herein, “encodes” or “encoding” refers to a DNA sequence which can be processed to generate an RNA and/or polypeptide.

The term “expand” as used herein, refers to an increase in number of cells or size, volume or area of a tissue construct. In certain embodiments, an engineered tissue construct expands, as determined by volume, weight, and area. In some embodiments, expansion of an engineered tissue construct is measured by an increase in overall cell number and/or entry into the cell cycle.

As used herein, “genetic construct” refers to an isolated polynucleotide which is introduced into a host cell. This construct may comprise any combination of deoxyribonucleotides, ribonucleotides, and/or modified nucleotides. In some embodiments, the construct is transcribed to form an RNA, wherein the RNA is translated to form a protein. This construct may be expressed in the cell, or isolated or synthetically produced. In some embodiments, the construct comprises a promoter, or other sequences which facilitate manipulation or expression of the construct.

As used herein, the term “growth factor” refers to a molecule that elicits a biological response to improve tissue regeneration, tissue growth or morphogenesis and organ function.

As used herein, the term “hepatocellular function” refers to a function or activity of a hepatic cell (e.g., a hepatocyte) characteristic of, or specific to, the function of liver parenchymal cells, e.g., liver-specific function. Hepatocellular functions include, but are not limited to albumin secretion, urea production, liver-specific transcription factor activity, metabolism, e.g., drug metabolism. In certain embodiments, the hepatocellular function is drug metabolism, for example, the enzymatic activity of human Phase I detoxification enzymes (e.g., cytochrome P450 activity), human Phase II conjugating enzymes, human Phase III transporters, and the like. For example coumarin 7-hydroxylation is a human-specific process mediated by human Phase I metabolic enzymes, e.g., CYP2A6 or CYP2A2, in response to known substrates and/or inducers. Hepatocellular function is also determined by measuring a “hepatocyte blood factor.” In certain embodiments, the hepatocyte blood factor is albumin, transferrin, alpha-1-antitrypsin, or fibronectin.

Maintenance of hepatocellular function can result from maintaining the desired morphology, cell-cell contact, environmental biochemical cues, adhesion, and the like, and within engineered tissue constructs described herein, can further result from promoting sufficient vascularization and oxygen and nutrient transport to the implanted construct.

The term “hydrogel” as used herein refers to a broad class of polymeric materials, that are natural or synthetic, which have an affinity for an aqueous medium, and is able to absorb large amounts of the aqueous medium, but which do not normally dissolve in the aqueous medium. Hydrogel is a type of biocompatible scaffold. Hydrogels can include, for example, at least 70% v/v water, at least 80% v/v water, at least 90% v/v water, at least 95%, 96%, 97%, 98% and even 99% or greater v/v water (or other aqueous solution). Hydrogels can comprise natural or synthetic polymers, the polymeric network often featuring a high degree of crosslinking. Hydrogels also possess a degree of flexibility very similar to natural tissue, due to their significant water content. Hydrogel are particularly useful in tissue engineering applications of the invention as scaffolds for culturing cells.

The term “hydrogel precursor” refers to any chemical compound that may be used to form a hydrogel. Examples of hydrogel precursors include, but are not limited to, a natural polymer, a hydrophilic monomer, a hydrophilic polymer, a hydrophilic copolymer formed from a monomer and a polymer.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic ligand conjugate).

As used herein, “in vitro” refers to processes performed or taking place outside of a living organism. In some embodiments, the processes are performed or take place in a culture dish.

As used herein, “in vivo” refers to processes that occur in a living organism.

As used herein, an “inducible element” includes an element that confers regulation on activity of a polypeptide and/or transcription of a downstream expressed region under inducing conditions. It may be obtained from enhancer regions that are also inducible. In some embodiments, the inducible element is an inducible promoter. In some embodiments, the inducible element is a multimerization domain.

The term “introducing” encompasses a variety of methods of introducing a genetic construct (e.g., DNA) into a cell, either in vitro or in vivo, such methods including transformation, transduction, transfection, and infection. Vectors are useful and preferred agents for introducing DNA encoding the interfering RNA molecules into cells. Possible vectors include plasmid vectors and viral vectors. Viral vectors include retroviral vectors, lentiviral vectors, or other vectors such as adenoviral vectors or adeno-associated vectors.

As used herein, the terms “linked,” “operably linked,” “fused”, or “fusion”, are used interchangeably. These terms refer to the joining together of two more elements or components or domains, by an appropriate means including chemical conjugation or recombinant DNA technology. Methods of chemical conjugation (e.g., using heterobifunctional crosslinking agents) are known in the art as are methods of recombinant DNA technology.

As used herein, the term “liver regeneration” refers to the expansion, growth, and increase in volume of the liver. Liver regeneration can occur with replacement of tissue loss with phenotypic fidelity of cell types (i.e., each cell type of the liver enters into proliferation to replace its own cellular compartment). Liver regeneration can also occur by replacement of tissue by activation of transdifferentiation pathways originating from stem cells. In certain embodiments, liver regeneration is deemed to have occurred by an increase in hepatocyte cell number, an increase in cell size, an increase in volume of the liver, and/or an increase in size of the liver and/or by an increase in production of a liver derived factor (e.g., HGF). See e.g., Michalopoulos (Comprehensive Physiology (2013), Vol. 3: 485-513), herein incorporated by reference.

The term “metabolic disorder” refers to a condition associated with aberrant glucose, lipid and/or protein metabolism and pathological consequences arising therefrom.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “non-parenchymal cells” as used herein, refers to the cells of or derived from the tissue surrounding or supporting parenchymal tissue in an organ or gland, for example, in a mammalian (e.g., human) organ or gland, or the connective tissue of such an organ or gland. Exemplary non-parenchymal cells include, but are not limited to, stromal cells (e.g., fibroblasts), endothelial cells, stellate cells, cholangiocytes (bile duct cells), Kupffer cells, pit cells, and the like. The choice of non-parenchymal cells used in the constructs described herein will depend upon the parenchymal cell types used. For example, a variety of both liver and non-liver derived non-parenchymal cells have been reported to induce hepatic function in co-culture.

As used herein, the terms “organogenesis” and “tissue organogenesis” refers to the design and development of engineered tissue or regenerated tissue. In some embodiments, organogenesis involves cell division, cell expansion, cell and tissue type differentiation and overall patterning of the tissue or organ.

As used herein, the term “parenchymal cells” refers to cells of, or derived from, the parenchyma of an organ or gland, e.g., a mammalian organ or gland. The parenchyma of an organ or gland is the functional tissue of the organ or gland, as distinguished from surrounding or supporting or connective tissue. As such, parenchymal cells are attributed with carrying out the particular function, or functions, of the organ or gland, often referred to in the art as “tissue-specific” function. Parenchymal cells include, but are not limited to, hepatocytes, pancreatic cells (alpha, beta, gamma, delta), myocytes, e.g., smooth muscle cells, cardiac myocytes, and the like, enterocytes, renal epithelial cells and other kidney cells, brain cell (neurons, astrocytes, glia cells), respiratory epithelial cells, stem cells, and blood cells (e.g., erythrocytes and lymphocytes), adult and embryonic stem cells, blood-brain barrier cells, adipocytes, splenocytes, osteoblasts, osteoclasts, and other parenchymal cell types known in the art.

Because parenchymal cells are responsible for tissue-specific function, parenchymal cells express or secrete certain tissue specific markers. In the liver, for example, liver tissue specific proteins include, but are not limited to, albumin, fibrinogen, transferrin, and cytokeratin 18 and cytokeratin 19. The functional activity of a particular parenchymal cell can vary with the type of non-parenchymal cell included within constructs described herein. For example, the quantity and rate of expression of albumin by hepatocytes in co-culture can vary between the type of fibroblast cell line used in a construct described herein.

Certain precursor cells can also be included as “parenchymal cells”, in particular, if they are committed to becoming the more differentiated cells described above, for example, liver progenitor cells, oval cells, adipocytes, osteoblasts, osteoclasts, myoblasts, stem cells (e.g., embryonic stem cells, hematopoietic stem cells, mesenchymal stem cells, endothelial stem cells, and the like). In some embodiments stem cells can be encapsulated and/or implanted under specified conditions such that they are induced to differentiate into a desired parenchymal cell type, for example, in the engineered tissue construct. It is also contemplated that parenchymal cells derived from cell lines can be used in the methodologies of the disclosure.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, “promoter” includes reference to a region of DNA that is involved in recognition and binding of an RNA polymerase and other proteins to initiate transcription. In one embodiment, the promoter is a Pol II promoter. Any Pol II promoter may be used in accordance with the present invention. In one embodiment, the Pol II promoter is a heat shock promoter. In another embodiment, the heat shock promoter is a minimal heat shock promoter. In a further embodiment, the minimal heat shock promoter is the Drosophila hsp70 minimal heat shock promoter.

As used herein, a “small molecule” is a molecule with a molecular weight below about 500 Daltons.

The term “stromal cell” refers to a type of cell in supporting tissue (e.g., connective tissue) that surrounds other tissues and organs. In some embodiments, stromal cells are non-hematopoietic, multipotent, self-renewable cells capable of trilineage differentiation. In some embodiments, stromal cells are fibroblasts.

The term “subject” as used herein refers to any living animal, a mammal, or a human.

As used herein, “substrate” refers to a surface or layer that underlies something, for example, a cell, cell culture, cell culture material, etc., or on which processes occur. In some embodiments, a substrate is a surface or material on which an organism lives, grows, and/or optionally obtains nourishment. The term “substrate” also refers to a surface or layer, e.g., a base surface or layer, on which another material is deposited. Exemplary substrates include, but are not limited to, glass, silicon, polymeric material, plastic (e.g., tissue culture plastic), etc. Substrates can be slides, chips, wells and the like.

The term “tissue” refers to a structure formed by related cells, with or without extracellular matrix, joined together, wherein the cells work together to accomplish specific functions.

As used herein, the term “vasculature” refers to an arrangement of blood vessels.

### Other Embodiments

The disclosure relates to the following embodiments. Throughout this section, the term embodiment is abbreviated as ‘E’ followed by an ordinal. For example, E1 is equivalent to Embodiment 1.
E1. A method for producing a multicellular tissue construct comprising the steps of:
   a. providing a biocompatible scaffold configured to support the construct;
   b. contacting the scaffold with feeder cells under conditions sufficient to populate the scaffold with the feeder cells;
   c. contacting the populated scaffold with tissue cells;
   d. co-culturing the tissue cells and feeder cells under conditions sufficient to allow the tissue cells to become established;
   e. selective removing the feeder cells; and
   f. culturing the cells remaining after step (e) under conditions sufficient to form the construct.
E2. The method of embodiment 1, wherein the scaffold comprises a hydrogel.
E3. The method of embodiment 1, wherein the feeder cells comprise fibroblasts.
E4. The method of embodiment 1, wherein step (c) further comprises contacting the scaffold with endothelial cells.
E5. The method of embodiment 1, wherein the feeder cells have been transduced with an apoptosis gene.
E6. The method of embodiment 5, wherein step (e) is performed by contacting the scaffold with an agent capable of inducing expression of the apoptosis gene, thereby causing apoptosis of the feeder cells.
E7. The method of embodiment 5, wherein the apoptosis gene comprises a caspase, thymidine kinase or cytosine deaminase.
E8. The method of embodiment 7, wherein the apoptosis gene comprises caspase9.
E9. The method of embodiment 6, wherein the agent is a small molecule.
E10. The method of embodiment 1, wherein the tissue construct is a mammalian organ.
E11. The method of embodiment 10, wherein the organ is a liver, heart, kidney or pancreas.
E12. A multicellular tissue construct produced by the method of any of the preceding embodiment.

### EQUIVALENTS AND SCOPE

Those skilled in the art will recognize, or be able to ascertain using no more than routine experimentation, many equivalents to the specific embodiments, in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments, in which exactly one member of the group is present in, employed in, or otherwise relevant to a given product or process. The disclosure includes embodiments, in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

### Example 1: Fibroblasts Expressing Inducible Caspase 9 are Activated by CID and Uniformly Eliminated by Apoptosis

Three-dimensional (3D) tissue engineered models have evolved to encompass a range of applications spanning therapeutic cell-based therapies to in vitro organoid models. Recapitulation of physiologic functions and native tissue behavior is key to studying and harnessing complex, tissue-specific phenomena in normal and pathophysiological states. A body of work has established that heterotypic and homotypic cell-cell interactions are of particular importance in engineered tissue. To study the temporal role that fibroblasts play in maintaining phenotype and function of an engineered tissue a cell line that could undergo quick, complete removal using a non-invasive trigger was developed.

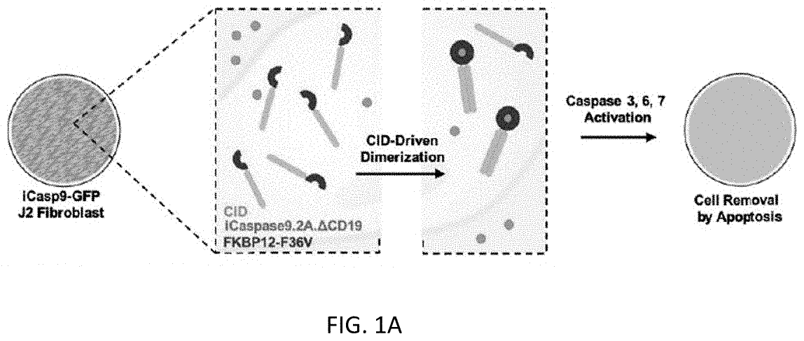

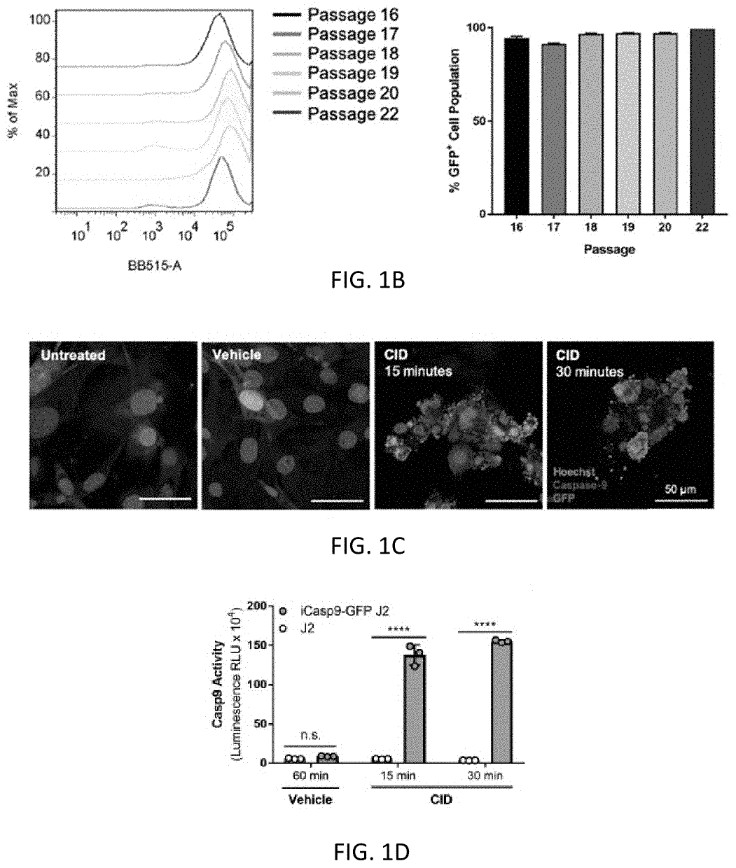

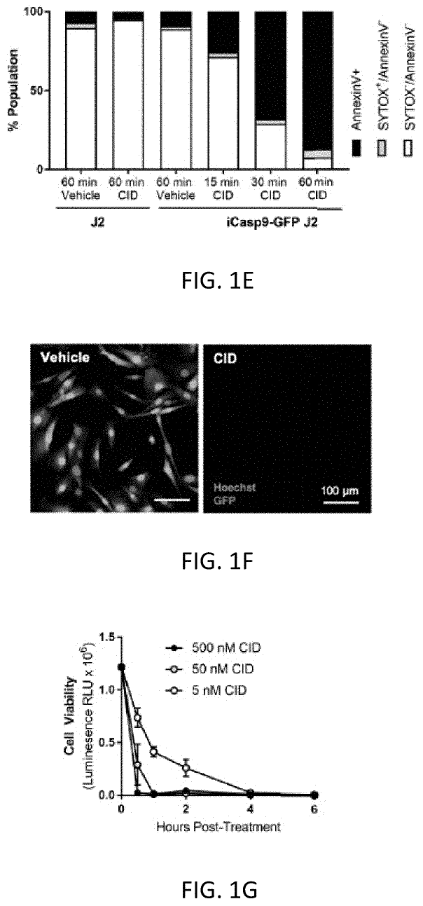

Inducible caspase-9 (iCasp9), which is activated by treatment with a small molecule chemical inducer of dimerization (CID; also known as rapalog, an analog of rapamycin) and leads to subsequent cell death through the intrinsic apoptosis pathway was utilized (FIG. 1A). The safety and efficacy of the iCasp9 transgene and CID have previously been shown in vitro and in vivo in animals and humans (A. Di Stasi, et al. (2011) dx.doi.org/10.1056/NEJMoa1106152; T. Gargett and M. P. Brown (2014) Front. Pharmacol. 5; K. C. Straathof, et al. (2005) Blood 105, 4247, each of which is herein incorporated by this reference). To generate inducible cells, 3T3-J2 fibroblasts (‘J2’) were transduced with lentivirus with a bidirectional expression cassette encoding iCasp9 and GFP genes (#15567 prvISCV-F-del Casp9.1RES.GFP; cloned to a lentivirus plasmid backbone with an SFFV promoter). FACS was used to enrich the infected population for the 15% highest-expression GFP+ cells. Flow cytometry analysis confirmed that the iCasp9-GFP J2s appeared homogeneous for at least 7 passages, and the population remained >97% GFP+ even at passage 20 (FIG. 1B). More specifically, J2s were lentivirally transduced using the 3rd generation lentiviral system with an iCasp9-IRES-GFP plasmid (Addgene; #15567 pMSCV-F-del 377 Casp9.IRES.GFP). Plasmids were co-transfected into HEK-293T cells with 378 pVSVG, pRSV-REV, and pMDLg/pRRE using a calcium phosphate transfection method. Assembled viruses were collected in the culture supernatant after 48 hours and precipitated using PEG-IT (SBI), resuspended in PBS, and stored at −80° C. To transfect J2s, virus was added to growth media and cultured overnight. iCasp9-GFP J2 fibroblasts at passage 22 were grown at confluence for 2 weeks to mimic experimental culture conditions, without a decrease in the percentage of the cell population with positive GFP expression. iCasp9-GFP J2s were plated in monolayer and dosed with ethanol vehicle or CID (B/B homodimerizer, AP20187; rapalog; Takara/ClonTech), and then assayed for cell viability using the CellTiter-Glo® Luminescent Cell Viability Assay (Promega), for caspase-9 activation using the CaspGLOW™ Fluorescein Active Caspase-9 Staining Kit (Thermo Fisher Scientific) and Caspase-Glo®9 Assay Systems (Promega), or stained with the Pacific Blue Annexin V/SYTOX™ AADvanced™ Apoptosis Kit (Thermo Fisher) to identify apoptotic cells by flow cytometry (>100,000 cells analyzed per condition).

After iCasp9-GFP J2 fibroblasts were exposed to CID, iCasp9 dimers were detected by staining with a caspase-9 antibody (FIG. 1C). Compared to wild-type J2s, iCasp9-GFP J2s underwent significantly increased caspase-9 cleavage at 15 (16-fold) and 30 (18-fold) minutes after CID dosing (FIG. 1D). To confirm that CID-triggered caspase-9 activation led to apoptosis, unfixed cells were stained with Annexin V, which binds to an early indicator of apoptosis, and SYTOX, a general marker of cell death, and analyzed by flow cytometry. The proportion of iCasp9-GFP J2s undergoing apoptosis increased in a time-dependent manner within the first hour after CID treatment (FIG. 1E). Lastly, CID-treated iCasp9-GFP J2s demonstrated efficient removal from culture (FIG. 1F) by 1 hour after exposure (FIG. 1G). Taken together, these results demonstrate that an iCasp9-bearing population of J2s could be treated with CID to quickly and efficiently eliminate them from culture by activating the apoptotic pathway.

### Example 2: Two-Dimensional Micropatterned Co-Cultures Depend on the Sustained Presence of Stromal Cells

Recapitulation of cues from the native hepatic microenvironment, including from cells, extracellular matrix, and soluble factors, has been found to lead to phenotypic rescue of primary hepatocytes as well as prolongation of longevity and function (P. Godoy et al. (2013) Arch. Toxicol. 87, 1315; A. A. Chen. Et a1. (2011) Proc. Natl. Acad. Sci. 108, 11842; G. H. Underhill, et al. (2016) Biomaterials 28, 256). In this system, the incorporation of J2 fibroblasts enhanced phenotypic stability of hepatocytes (S. R. Khetani and S. N. Bhatia (2018) Nat. Biotechnol. 26, 120; C. Y. Li et al. (2014) Tissue Eng. Part A. 20, 2200; E. E. Hui and S. N. Bhatia (2007) Proc. Natl. Acad. Sci. 104, 5722). To study this phenomenon, a previously engineered actuatable 2D platform to enable the manipulation of established co-cultures was employed. Using this platform, the dependency of hepatocytes on fibroblasts was interrogated in 2D; it was found that despite an initial priming phase of direct cell-cell contact with fibroblasts, primary human hepatocytes did not maintain phenotypic stability if fibroblast juxtacrine and paracrine support were both removed (E. E. Hui and S. N. Bhatia (2007) JoVE J. e268). To investigate if controlled apoptosis in multicellular tissues for engineered organogenesis (CAMEO)-driven removal of fibroblast support would be disruptive to hepatocyte culture in 2D, primary human hepatocytes and J2 fibroblasts were cultured in a micropatterned co-culture (MPCC).

Specifically, MPCC were fabricated as described previously (S. R. Khetani and S. N. Bhatia (2005) Nat. Biotechnol 26, 120; S. March et al. (2015) Nat. Protoc. 10, 2007). Briefly, collagen was adsorbed in each well of a 96 well plate (glass bottom), and then patterned using an elastomeric polydimethysiloxane mold and oxygen plasma gas ablation. Human hepatocytes were thawed and seeded (70 k/well) on the collagen islands (500 μm with 1,200 μm center-to-center spacing). Adhered hepatocytes (˜10 k/well) were allowed to spread overnight before fibroblasts were seeded for co-culture (7 k/well). Supernatant was collected from cultures every other day and stored at −20° C. Human albumin was quantified using an enzyme-linked immunosorbent assay using a sheep anti-rat albumin antibody (ELISA) (Bethyl Laboratories) and 3,3′,5,5′-tetramethylbenzidine (TMB, Thermo Fisher). Urea concentration was measured using a colorimetric (diacetylmonoxime) assay with acid and heat (Stanbio Labs). CYP3A4 activity was assessed with the luminogenic P450-Glo™ CYP450 assay kit (Promega) for nonlytic assays using cultured cells. Cultures were pre-treated with 25 μM rifampin or 1:1000 DMSO vehicle control prepared in hepatocyte maintenance media for 72 hours (daily replenishment) where indicated.

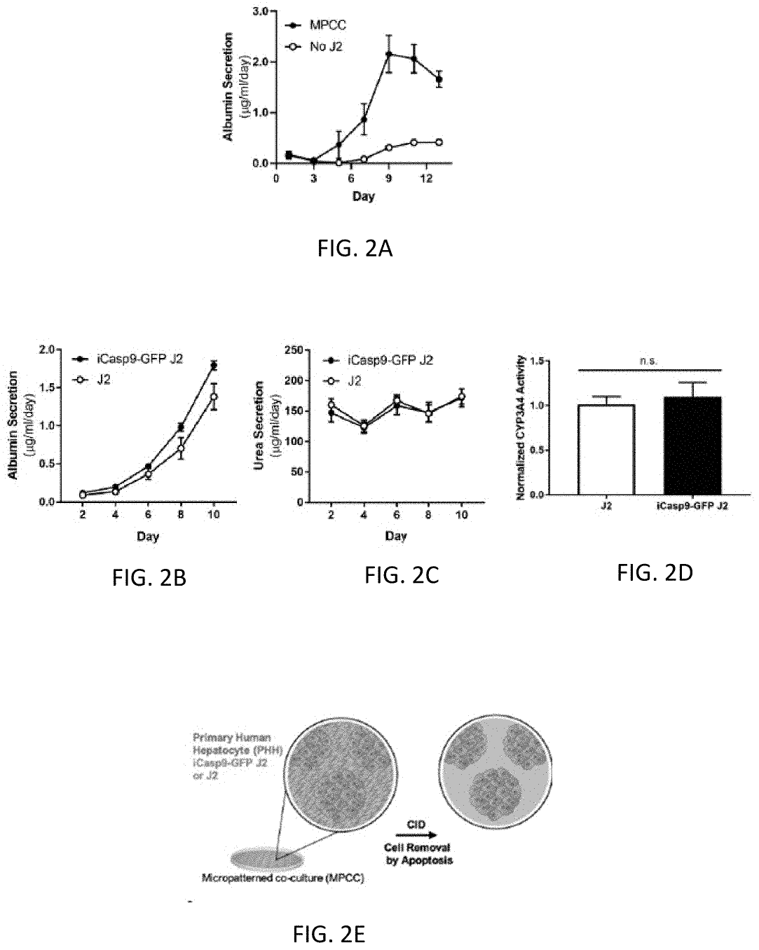

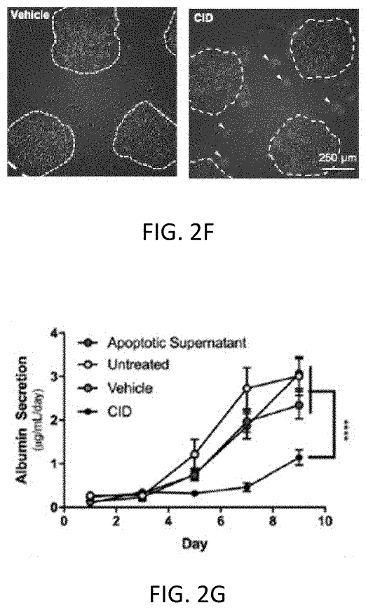

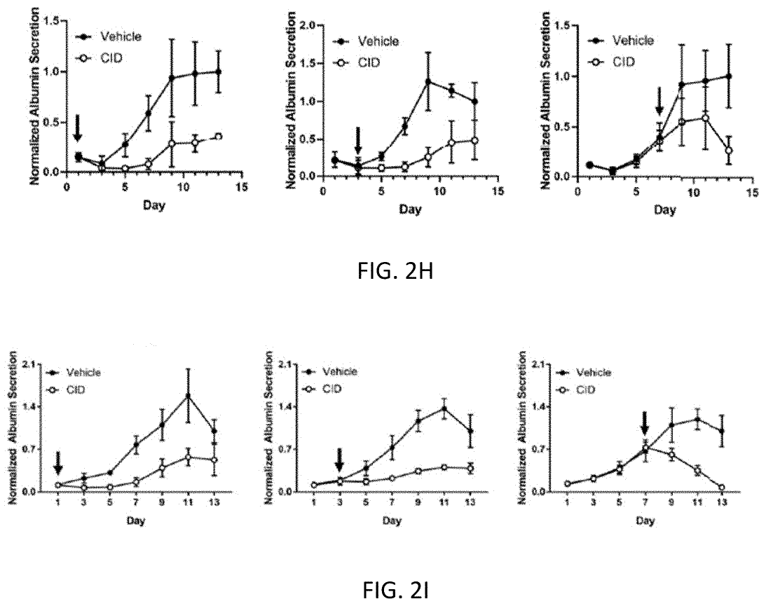

The results confirmed that hepatocyte phenotypic stability is enhanced by J2 co-culture (FIG. 2A). iCasp9-GFP J2 and J2 fibroblasts both provided support of multiple axes of liver function, including synthesis of albumin protein (FIG. 2B), production of urea as a byproduct of nitrogen metabolism (FIG. 2C), and expression of drug metabolism-related enzymes (FIG. 2D), suggesting that genetic modification of J2s did not abrogate their capability to support hepatocytes.

To eliminate inducible apoptosis gene-bearing cells in a multicellular culture, iCasp9-GFP J2-bearing MPCCs were treated with 50 nM CID (FIG. 2E). CID-dosed MPCCs displayed selective removal of the iCasp9-GFP J2 population (FIG. 2F), whereas unmodified J2s plated in MPCCs were unaffected by CID exposure (data not shown). Furthermore, hepatocyte albumin production was not abrogated if MPCCs were cultured with conditioned ‘apoptotic’ medium (media collected from cells cultured with 50 nM CID for 2 hours), suggesting that at least one axis of liver-specific function was not affected by exposure to neighboring apoptotic cells (FIG. 2G). Altogether, these data suggest that CID-driven removal of stromal cells by apoptosis is a compatible system for probing phenotypic stability of hepatocytes in MPCCs.

To query the dependence of hepatocytes on fibroblasts, fibroblasts were deleted from MPCCs by CID treatment at various time points and albumin production was assessed as a surrogate marker of phenotypic stability. Deletion of stromal cells resulted in loss of hepatocyte phenotypic stability at early (day 1), intermediate (day 3), and late (day 7) time points (FIGS. 2H and 2I). Taken together, these data suggest that the function of primary human hepatocytes is heavily reliant on stromal support in this 2D MPCC configuration, which is consistent with past 2D studies (E. E. Hui and S. N. Bhatia (2007) Proc. Natl. Acad. Sci. 104, 5722; S. R. Khetani and S. N. Bhatia (2008) Nat. Biotechnol. 26, 120).

### Example 3: Fibroblasts Expressing Inducible Caspase 9 can be Eliminated from Three-Dimensional Multicellular Spheroid-Laden Hydrogels

Previous work identified that phenotypic stability and longevity of primary hepatocytes cultured as 3D microtissues were transiently supported by pre-aggregation to increase homotypic cell-cell interactions, and were further enhanced upon inclusion of J2 fibroblasts (C. Y. Li et al. (2014) Tissue Eng. Part A 20, 2200). Thus, iCasp9-GFP J2 fibroblasts were incorporated into these 3D hepatic ensembles, which were fabricated by plating primary human hepatocytes and fibroblasts in microwells in order to facilitate physical cell-cell contacts, as previously described (K. Stevens et al. (2013) Nat. Commun. 4, 1847; K. R. Stevens et al. (2017) Sci. Transl. Med. 9, eaaah5505). Specifically, cryopreserved 365 human hepatocytes were thawed and immediately plated with fibroblasts in AggreWells (400 μm pyramidal microwells) and incubated overnight. Hepatic spheroids (about 150 hepatocytes per spheroid, ˜100 μm diameter) were imaged and analyzed to quantify the extent of spheroid compaction. Individual spheroids were isolated manually using Fiji, (J. Schindelin et al. (2012) Nat. Methods 9, 676). Fiji was used to uniformly adjust brightness/contrast, pseudocolor, and merge images. Spheroid-laden hydrogels were imaged on a Zeiss confocal microscope using a water immersion 40× objective or the Leica SP8 spectral confocal microscope using the 10× air or 25× water immersion objective. Live imaging was captured using a Nikon Spinning-disk Confocal Microscope with TIRF module. Greyscale erosion was applied to threshold for hepatocytes (˜7 μm). Resulting morphologies were traced and measured for circularity. Resulting spheroids were embedded in fibrin (10 mg/mL bovine fibrinogen, 1.25 U/mL human thrombin; Sigma-Aldrich) using 96 microwell plates as molds. Spheroid-laden hydrogels were cultured in hepatocyte media supplemented with 10 μg/mL aprotinin, a serine protease inhibitor, to prevent hydrogel degradation.

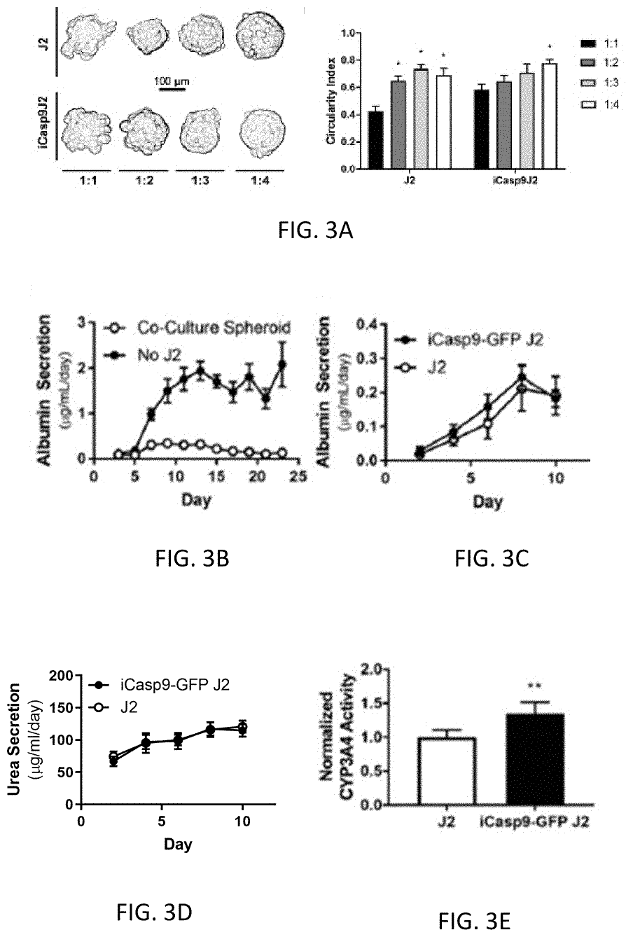

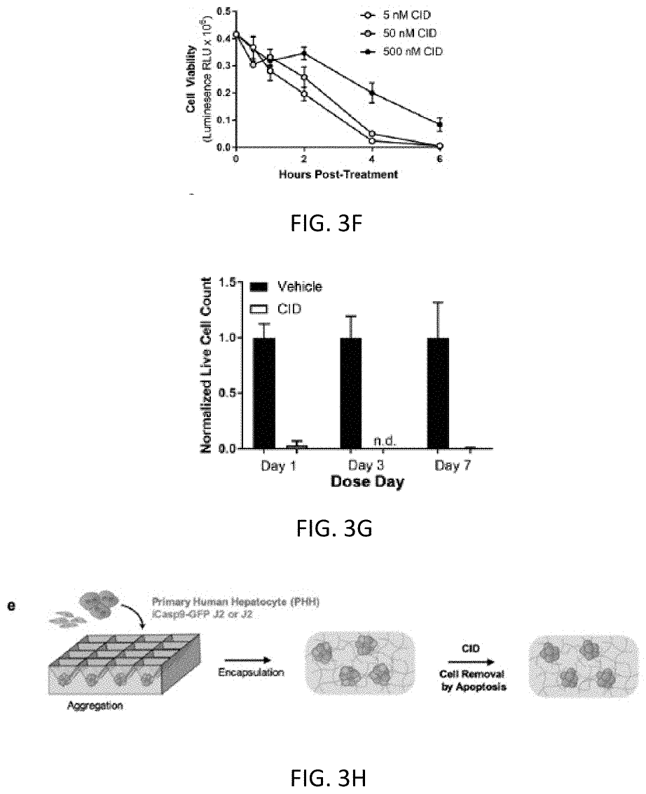

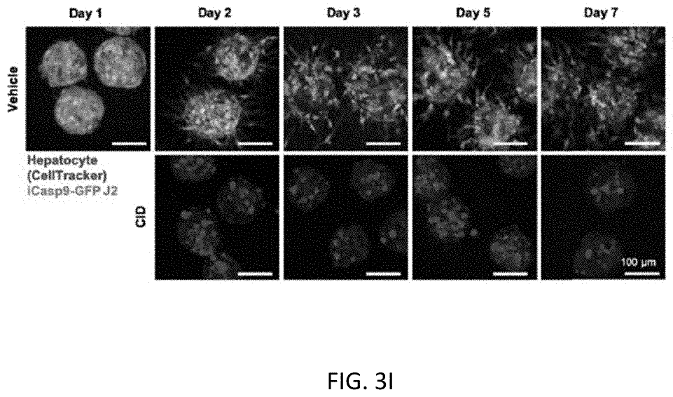

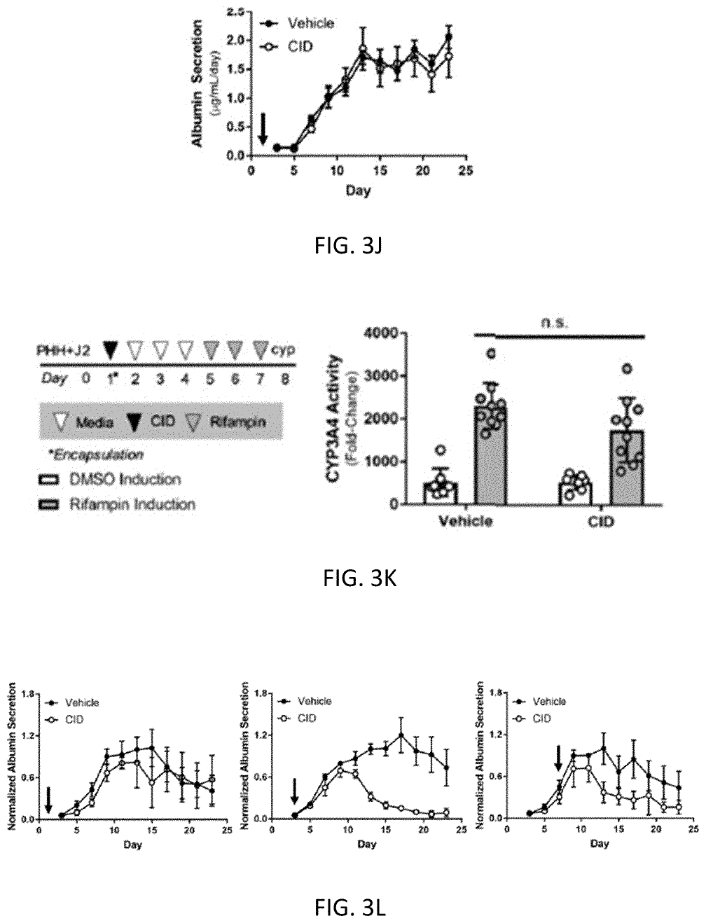

Optimal overnight aggregation into stable spheroids was achieved by increasing the amount of fibroblasts co-seeded in the microwells (FIG. 3A). Resulting spheroids were encapsulated in a 10 mg/mL fibrin hydrogel (crosslinked with 1.25 U/mL thrombin). Fibroblast co-culture, which provided supportive cell-cell interactions and increased aggregation stability, significantly improved the rate of primary human hepatocyte albumin secretion from the ensembles (FIG. 3B). Spheroid-laden hydrogels containing either J2s or iCasp9-GFP J2s cultured at a 1:4 (hepatocyte:fibroblast) ratio both exhibited enhanced synthetic (albumin production; FIG. 3C), metabolic (nitrogen metabolism; FIG. 3D), and detoxification (CYP3A4 activity; FIG. 3E) functions of hepatocytes.

To confirm the effectiveness of CID treatment for elimination of iCasp9-GFP J2 fibroblasts embedded in a hydrogel encapsulated iCasp9-GFP fibroblasts were treated with CID. Fibroblast viability was undetectable after 6 hours (FIG. 3F). Furthermore, fibroblasts did not regrow over the course of 21 days (FIG. 3G). Next, to assess the specificity of CAMEO in 3D, hydrogel-encapsulated, multicellular spheroids (in which iCasp9-bearing fibroblasts were placed in close proximity to hepatocytes) were cultured and the ensembles were treated with CID in an attempt to specifically eliminate iCasp9-GFP fibroblasts (FIG. 3H). In these spheroid-laden hydrogel cultures, CID was able to access iCasp9-GFP J2s, leading to their robust and specific deletion throughout the hydrogel, without any apparent toxicity to co-cultured hepatocytes (data not shown).

To probe the dependence on trapped factors from the fibroblast population, co-cultures were dosed with CID prior to encapsulation. Apoptotic debris and conditioned supernatant were removed to account for remaining fibroblast factors. No difference in hepatocyte function was observed when CID was administered pre- or post-encapsulation in fibrin demonstrating that the observed hepatocyte stability was not due to retained fibroblast-derived factors (data not shown). While the retention of fibroblast-derived factors was not necessary to maintain cultures, the cell-cell and cell-matrix interactions were probed for their importance in promoting hepatocyte phenotypic stability. To perturb the cell-cell and cell-matrix interactions, function-blocking antibodies against human B1 integrin or human E-cadherin were added to the culture before culturing with fibroblasts (pre-compaction), or after co-culture and encapsulation in fibrin (post-compaction). Functional blockage pre-compaction reduced albumin secretion in hepatocytes whereas no difference was observed in treatment post-compaction demonstrating cell-cell and cell-matrix adhesion was essential for hepatocyte cultures (data not shown).

These results suggest that CAMEO can be employed by dosing embedded co-cultures with CID to trigger the removal of inducible apoptosis gene-bearing cells in 3D multicellular ensembles.

### Example 4: Fibroblasts are not Required to Maintain Hepatocyte Function in Three-Dimensional Spheroid-Laden Cultures

To probe the dependence of hepatocyte phenotypic stability on fibroblast co-culture in 3D, fibroblasts were deleted from spheroid-laden hydrogel cultures using CAMEO after 1 day of hepatocyte-fibroblast co-culture. Fibroblasts were robustly removed from 3D co-cultures as described in the above Examples, and detected by immunofluorescence imaging (FIG. 3I). While 2D MPCC cultures were found to be dependent on fibroblast interactions for the extent of the experiment (1.5 weeks, FIG. 2H), fibroblast-depleted 3D cultures exhibited stable phenotype, as detected by albumin secretion rate, for up to 3 weeks (FIG. 3J). Furthermore, fibroblast-depleted and fibroblast-intact cultures underwent similar induction of CYP3A4 activity in response to a 72-hour rifampin treatment (2504) starting on day 5 (FIG. 3K). Notably, when 50 nM CID-triggered iCasp9-GFP J2 deletion was delayed until later time points (after 3 or 7 days of hepatocyte-fibroblast co-culture), hepatocyte function was negatively impacted, suggesting that primary hepatocytes cultured with J2s and embedded in fibrin are sensitive to deletion kinetics (FIG. 3L). Taken together, these findings, enabled by CAMEO, demonstrate that there is a window of opportunity for fibroblast deletion in this particular tissue engineered context.

### Example 5: Fibroblasts Enhance Self-Assembly of a Perfused Vasculature in Microfluidic Devices

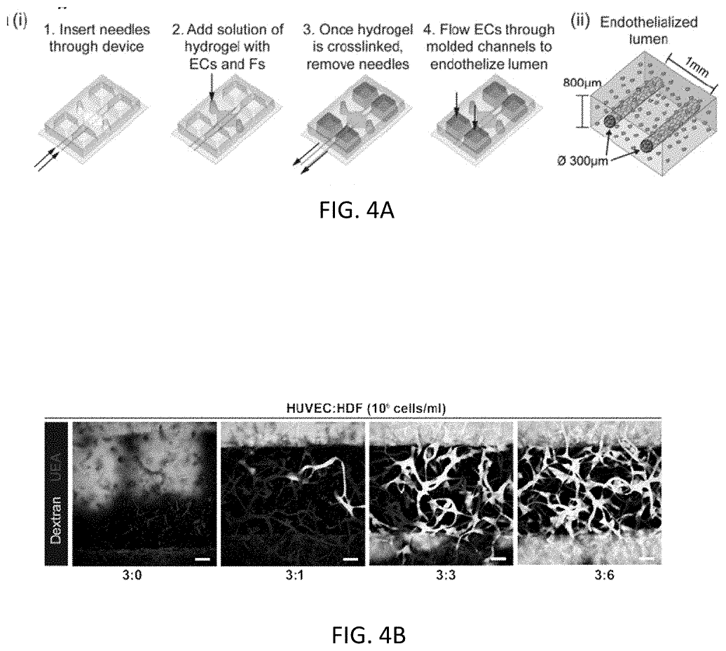

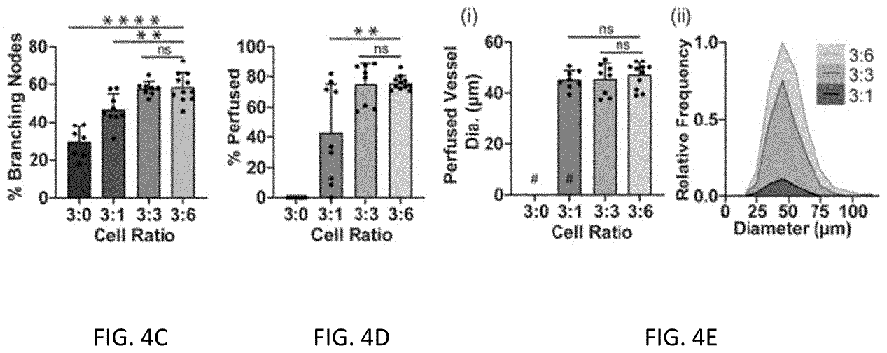

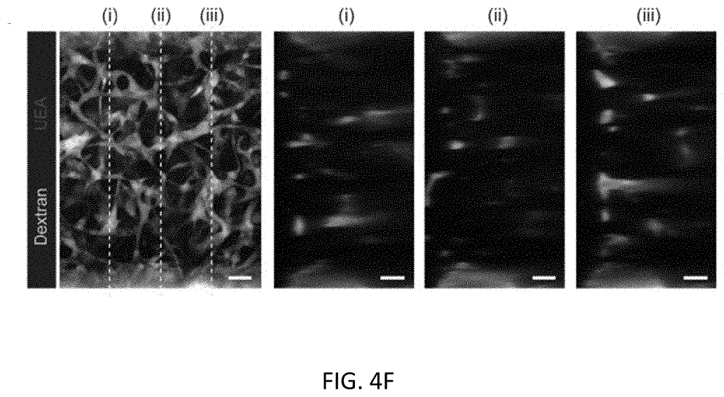

To further investigate the role of fibroblasts, a system that enables the 3D morphogenesis of an endothelial cell and fibroblast co-culture to result in a self-assembled, perfusable vascular network connected to microfluidic channels was established. Building on a previously reported device that was originally designed to study angiogenesis in vitro, (A. Di Stasi. et al. (2011) http://dx.doi.org/10.1056/NEJMoa1106152; T. Gargett and M. P. Brown, (2014) Front. Pharmacol, 5), a new iteration that supports vasculogenesis was developed (D. H. T. Nguyen et al. (2013) Proc. Natl. Acad. Sci. U.S.A. 110, 6712; B. Trappmann et al. (2017) Nat. Commun. 8, 371). This microfluidic device consists of a polydimethylsiloxane (PDMS) silicone-based mold bonded to a glass coverslip that features a central tissue chamber (cells plus extracellular matrix) through which two endothelial cell-lined vessels are perfused. To achieve this arrangement, briefly, two guides on the side of the PDMS device were designed for the insertion of two parallel needles (300 μm in diameter and 1 mm apart) that traverse the central chamber (FIG. 4A).

More specifically, the molds for the 2-channel microfluidic devices were constructed using stereolithography (Proto Labs). Polydimethylsiloxane (PDMS) was cured at a standard mixing ratio overnight at 60° C. in the mold, and individual devices were cut and plasma-bonded to glass slides. To enhance ECM bonding to PDMS, the surface inside the tissue chamber of the devices was functionalized with 0.01% poly-L-lysine and 1% glutaraldehyde following plasma-activation and washed overnight in DI water. On the day of seeding, devices were soaked in 70% ethanol (EtOH) and dried. Acupuncture needles (300 μm diameter) (Hwato) were blocked in 0.1% (w/v) bovine serum albumin (BSA) (Sigma) in phosphate buffer saline (PBS) for 45 minutes and inserted through the two needle guides. Devices with needles were UV-sterilized for 15 minutes. Once the needles were inserted, a fibrinogen solution containing human umbilical vein endothelial cells (HUVECs) and growth-arrested human dermal fibroblasts (HDFs) were added into the tissue chamber with thrombin. To prepare the cells for seeding, HDFs or iCasp-9 HDFs were growth-arrested with 10 μg/mL mitomycin in FGM-2 for 2.5 hours and thoroughly washed 5 times in FBM. Both HUVECs and HDFs were lifted from culture plates using TrypLE Express (Gibco), centrifuged at 200 g for 4 minutes, and resuspended to a concentration of 20 million cells/mL in EGM-2. A solution of HUVECs (3 million cells/mL), HDFs or iCasp-9 HDFs (0, 1 million, 3 million, or 6 million cells/mL), fibrinogen (2.5 mg/mL), thrombin (1 U/mL) in EGM-2 was prepared for the bulk hydrogel region of each device. After the addition of thrombin, the solution was quickly injected into the tissue chamber, and the devices were repeatedly rotated while the solution crosslinked. Appropriate media was added to each well of the device, and the devices were placed in the incubator (37° C., 5% CO₂). After 15 minutes, the needles were carefully removed from the devices to create 300 μm hollow channels between the wells. Each channel of the device was seeded with additional HUVECs at 2 million cells/ml for at least 5 minutes on each side (top and bottom) in the incubator. Each device received 200 μl of appropriate media daily and cultured on the rocker inside the incubator. Once the fibrinogen polymerized into fibrin, the needles were removed to create two hollow, microfluidic channels. These channels were then seeded with additional HUVECs to line the walls in a monolayer. This procedure resulted in the formation of two parallel, endothelialized vessels that could be instantly perfused with media and provide nutrients to the cells in the surrounding ECM.

To understand how different densities of fibroblasts would affect the vascular morphogenesis of a given number of endothelial cells, HUVECs (3 million cells/ml) were co-cultured with various concentrations of HDFs (0, 1, 3, and 6 million cells/ml) for 7 days in the fibrin surrounding the needle-molded endothelial channels (FIG. 4B). Max projection images s were assembled and processed using Imaris 9.2.1 (Bitplane). Intensity of stack images was depth compensated by utilizing a built-in autocorrelation correction Matlab plug-in (MathWorks). The dextran channels were smoothed using the Gaussian Filter that used a 3.89 μm filter width in order to make perfused channels more clearly visible in figures. Thresholding and gamma correction were applied to all images. False color was applied to both the UEA lectin and TRITC—Dextran were false-colored to red and cyan, respectively. Only unprocessed images were used in the image analysis, explained above. For images containing hepatocytes, false coloring was applied to UEA lectin, FITC Dextran and Argl as red, cyan, and green, respectively. Uniform volumetric masks were applied that were thresholded to denoise images due to antibody aggregation and fluorophore bleed through. These corrections were applied to the Dextran and Argl channels in all hepatocyte images.

HUVECs alone were able to make branching networks suggestive of functional vasculature (FIG. 4C), but when perfused with a fluorophore-conjugated dextran solution (500 kDa) introduced to one of the microfluidic channels, these structures were unable to transport dextran. Only when HDFs were added to the HUVEC culture were highly branched networks that were able to transport the dextran solution through the self-assembled vessels observed. Furthermore, co-cultures with higher densities of HDFs (3:3 and 3:6 HUVEC:HDF) yielded the highest average percentage of perfused vessels (FIG. 4D). The average diameters of perfused self-assembled vessels did not change with different densities of HDFs (45 μm) (FIG. 4E), but increasing the density of fibroblasts resulted in a higher density of perfusable vasculature as well as a wider distribution of perfused vessel diameters (FIG. 4E). At the highest concentration of HDFs (3:6 ratio), the engineered tissue was well vascularized all throughout its thickness after the 7-day co-culture (FIG. 4F), which prompted the use of this ratio for the rest of the study.

Vessel networks were analyzed by fixing all devices with 4% PFA for 30 minutes on a rocker and washing with PBS overnight at 4° C. The devices were then blocked in 3% BSA overnight at 4° C. Lectin (UEA DyLight 649, Vector Labs) was diluted in the blocking solution at 1:100 dilution and added to the devices for another 4° C. overnight incubation. Devices were then washed overnight with PBS at 4° C. and kept in PBS with 0.02% (w/v) sodium azide at 4° C. until imaging. Before imaging, a solution of 500 kDa fluorescein isothiocyanate (FITC)- or tetramethylrhodamine isothiocyanate (TRITC)-conjugated dextran (0.15 mg/ml) was added to one of the microfluidic channels to generate gravity-driven hydrostatic pressure between the two channels of the device. All device images were then captured by a Leica SP8 confocal microscope (Leica, Wetzlar, Germany) using either a Leica 10×/0.30NA W U-V-I WD-3.60 Water or 25×/0.95NA W VISIR WD-2.50 Water objective and Leica LAS X imaging software. Within experimental runs, the same laser intensities and settings were applied to all samples.

A custom MATLAB script was used to study properties of the vascular network. The script imports a .tif image stack with a surface marker in one channel, in this case lectin, and the fluorescent dye in another, FITC- or TRITC-dextran. The aspect ratio of each image is adjusted so that each voxel is equally spaced in all directions. Since the image resolution is higher in-plane (x- and y-axes) than along the depth (z-axis), this step typically results in a reduction in the number of pixels in the plane and an increase in the number of stack layers. The 3D volume of the cell surface marker channel is then smoothed using a spatial-domain Gaussian kernel to reduce noise in the Lectin channel. The script relies on user inputs to generate binary images of the complete vascular network from the Lectin signal, and the perfused network according to the FITC-Dextran. First, the user selects a 2D region of interest between the two needle-molded channels on the device, and performs contrast-limited adaptive histogram equalization (CLAHE) on both signals for each z slice. This step is performed to overcome a substantial loss in signal with depth of the volume. The user then specifies a luminance threshold for each channel and is able to move between different images in the z stack to best capture the vessel structure and perfusion of FITC-Dextran. For the fluorescent dye, the signal is converted using only this specified uniform threshold to capture the regions of perfusion. For the Lectin channel, which exhibits poorer contrast, the user first selects a liberal threshold to segment the 3D volume. The script refines this initial segmentation for each slice using distance regularized level set evolution (DRLSE) with a double well potential (see full code for setup of the iterative segmentation refinement). The DRLSE-segmented slices are merged to form the final volume of the full vessel network. Any holes in the Lectin volume are filled and islands smaller than ˜(50 μm)³ are removed. From the identified Lectin network, a 3D medial axis skeleton is generated using homotopic thinning. This is further converted into a graph with links representing the vessel structures, and nodes to identify branching and terminal points of the vessels. Links are further characterized as perfused and non-perfused vessels based on the overlap of the binarized FITC-Dextran with the vessel network. Links with <25% perfusion are classified as non-perfused links. The average length of all links from the Lectin skeleton is reported. The vessel radii are calculated based on the distance between the membrane skeleton and medial skeleton of the graph links, and the mean diameters for all vessels, as well as only perfused vessels, are reported. The percentages reported for branching nodes represent the fraction of branching nodes out of all nodes. The user can toggle through the z-stack of the binary channels overlaid on the original images, as well as overlaid on each other to confirm accuracy.

To visualize the process of vasculogenesis in the microfluidic devices, GFP-labeled HUVECs were seeded in the needle-molded microfluidic channels to distinguish them from the non-labeled HUVECs in the bulk fibrin gel. Fixing samples at different times and staining with Ulex Europaeus Agglutinin (UEA) lectin (which labels all HUVECs) allowed monitoring of the process of vascular network formation.

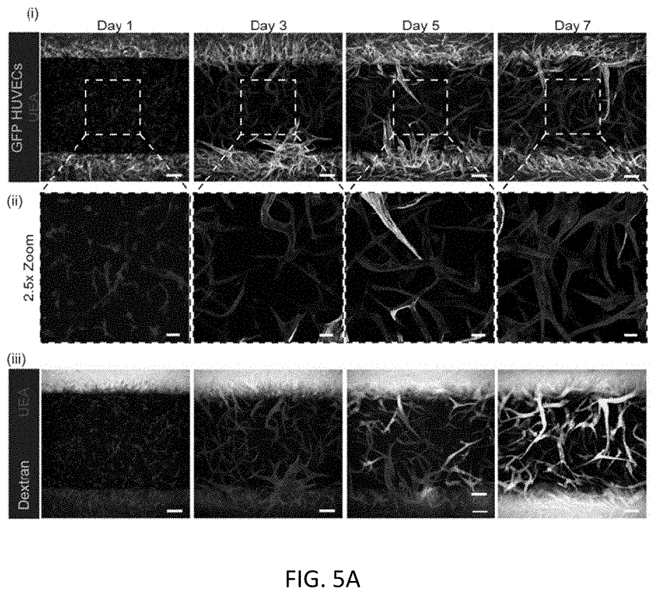

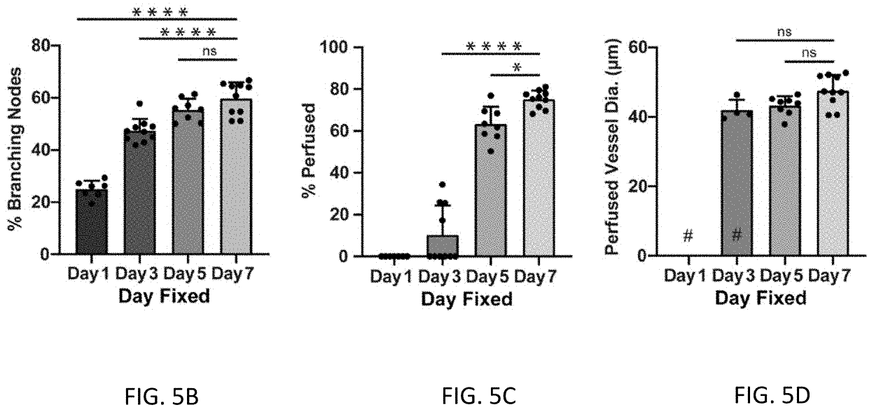

As early as day 1, GFP-HUVECs with individual protrusions that start to form intercellular connections were observed (FIG. 5A). After 3 days in culture, some HUVECs from the microfluidic channels (GFP-labeled) had migrated into the bulk gel and formed chimeric networks with the (unlabeled) HUVECs in the bulk ECM (FIG. 5B). Some of these self-assembled structures became perfusable by day 3, and the percentage of perfused vessels continued to increase over time, achieving ˜80% by day 7 (FIG. 5C). Interestingly, the average diameters of the perfused vessels in the network remained steady after day 3 (FIG. 5D). Together, these data suggest that the vascularization process inside the devices involves both the self-assembly of the bulk HUVECs, and invasion of HUVECs from the microfluidic channels to form a fully interconnected perfusable vasculature having both the originally templated, larger channels and the self-assembled vasculogenic network.

### Example 6: CAMEO Allows for a Selective Removal of Fibroblasts from Co-Culture

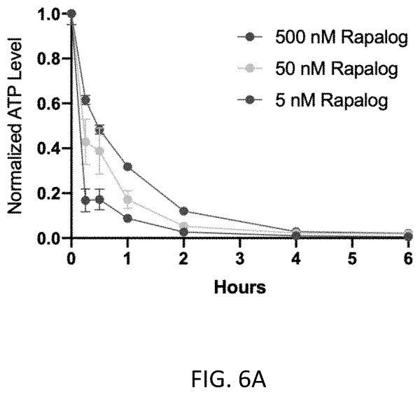

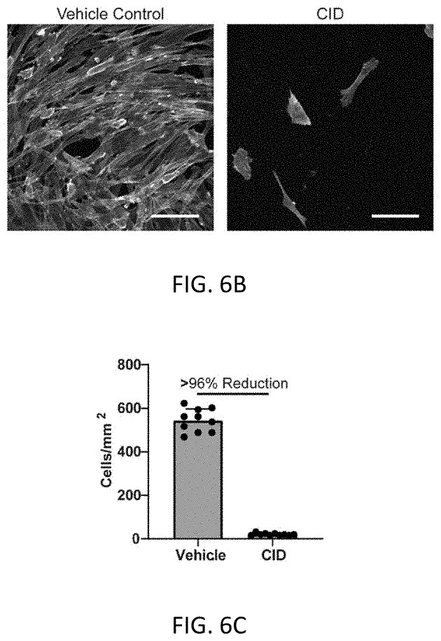

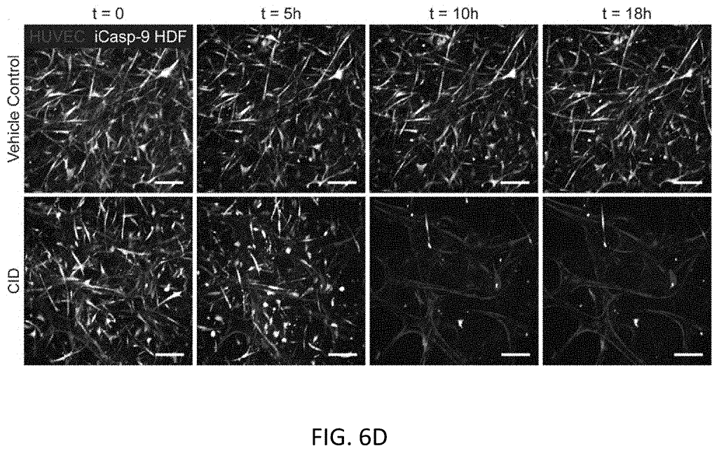

To be able to remove fibroblasts on demand from the developing co-culture, the synthetically engineered, inducibly activated caspase-9 (iCasp9) outlined in the examples above was used. Using this inducible transgene, iCasp9-HDFs were established and characterized for effectiveness. iCasp9-HDFs were seeded on tissue culture plastic, and allowed to form a confluent monolayer before adding different concentrations of CID. Even at the lowest concentration of CID used (5 nM), the levels of cellular ATP dropped precipitously within 2-4 hours, suggesting the onset of apoptosis (FIG. 6A). In addition, the rapidity of ATP loss increased with increasing CID concentration. Overnight treatment of 10 nM CID resulted in an average of ˜96% reduction of iCasp9-HDFs in culture compared to the vehicle control (0.002% ethanol) (FIGS. 6B and 6C).

To examine the temporal and selective control of the iCasp9-HDFs in a 3D system, mRuby-LifeAct-HUVECs with iCasp9-HDFs (GFP) were co-cultured in the microfluidic platform. After 7 days of co-culture, the cells formed a robust vasculature network as previously observed above, and then 10 nM of CID (or the vehicle control) was added to the media. Live imagining was used to monitor the response to CID. Specifically, microfluidic devices with LifeAct-Ruby HUVECs and iCasp9-HDFs (GFP) were made and cultured for 7 days as described above. Then, the devices were transferred to a custom-made Petri dish that limits media evaporation. Media was changed to EGM-2 containing OxyFluor (Oxyrase) at 1:100 dilution. Right before the start of imaging, either CID (10 nM) or vehicle was added to the devices and the petri dish was transferred into the microscope environmental chamber preconditioned to 37° C., 5% CO₂, and 100% humidity. A 150 μm stack was imaged in each device by a Yokogawa CSU-10/Zeiss Axiovert 200M inverted spinning-disk microscope using a Zeiss 10×/0.45NA Air objective every 30 minutes for 18 hours. The capture was automated using Metamorph 7.8.9.0 (Molecular Devices).

CID treatment induced rapid rounding iCasp9-HDFs by 5 hours, and most of the fibroblast population was removed by 10 hours (FIG. 6D). Interestingly, the rounding event was preceded by a pulse of contraction of the fibroblasts and the surrounding tissue (data not shown), consistent with reported caspase-induced ROCK1 activation and ROCK1-mediated actin-myosin contraction (K. C. Streethof, et al. (2005) Blood 105, 4247; S. R. Khetani and S. N. Bhatia (2008) Nat. Biotechnol. 26, 120). Acute effects of this massive elimination of HDFs was not observed despite being in some cases in direct contact with the dying cells, or any noticeable deterioration of the vascular structure.

### Example 7: Transient Co-Culture with Fibroblasts is Sufficient to Support Vasculogenesis

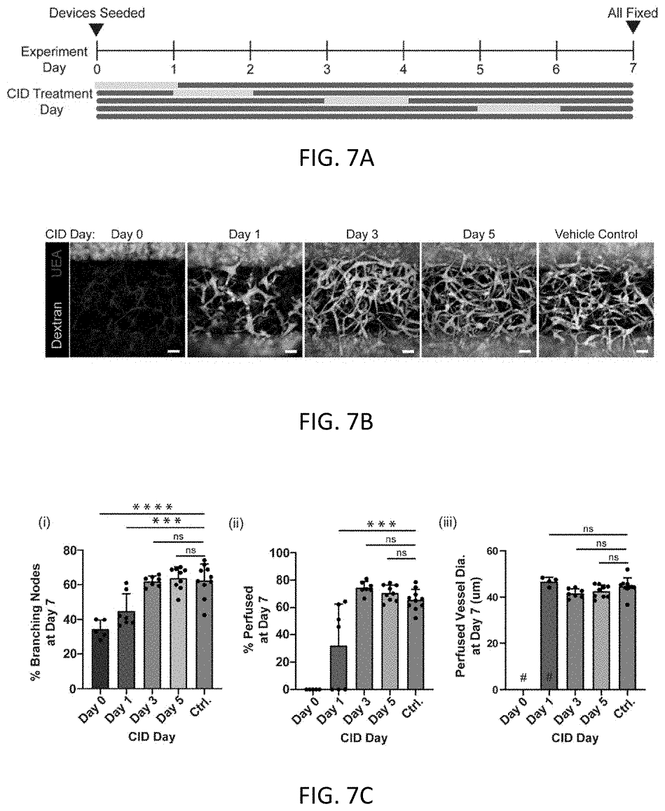

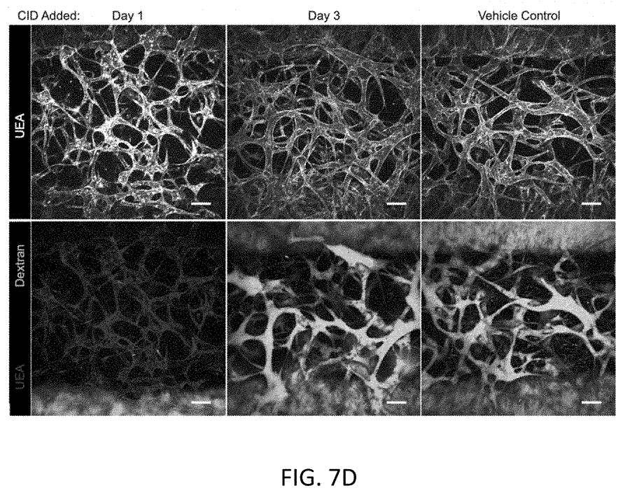

To understand the dependence of endothelial cells on fibroblasts for vasculogenesis, HUVECs were co-cultured with iCasp9-HDFs in microfluidic devices and HDFs were removed with 50 nM CID at different time points (day 0, 1, 3, and 5) during the 7-day culture period (FIG. 7A). When HDFs were removed within an hour after the completion of the device seeding (day 0), HUVECs were still able to form a network by day 7, but the resulting structure was minimally interconnected (FIGS. 7B and 7C), and none of these structures could transport dextran, indicating incomplete vascular morphogenesis similar to HUVEC-only cultures (FIG. 7C). Removing HDFs at day 1 resulted in a few perfusable structures by day 7, but most devices still showed little to no perfusion. Removing HDFs at day 3 or day 5 yielded highly interconnected and perfusable endothelial networks that were comparable to the control networks in which HDFs were never removed. The average diameter of the perfused vessels was comparable as well for all conditions except the day 0-deletion condition, though the distribution of the vessel diameters in the day 1-deletion condition was more dispersed. Similar observations were also made with iCasp9-transduced human lung fibroblasts (iCasp9-HLFs) where the HUVECs were able to self-assemble into perfusable vasculature with only 3 days of co-culture with iCasp9-HLFs (FIG. 7D), suggesting that this short-lived dependency on fibroblast support is not specific to HDFs. All together, these data suggested that the presence of fibroblasts during the first 1-3 days of co-culture is critical for the functional vasculogenesis of endothelial cells, but the fibroblasts can be removed thereafter without structurally affecting the resulting vasculature.

### Example 8: CAMEO Enhances Function of Vascularized, Engineered Hepatic Tissues

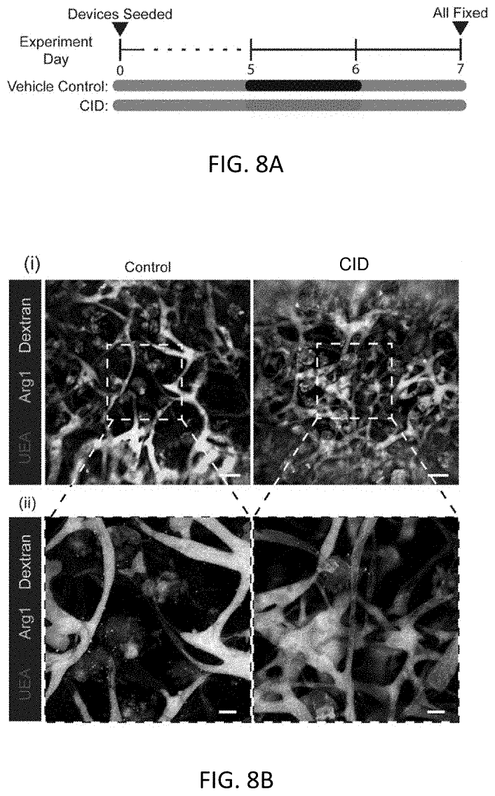

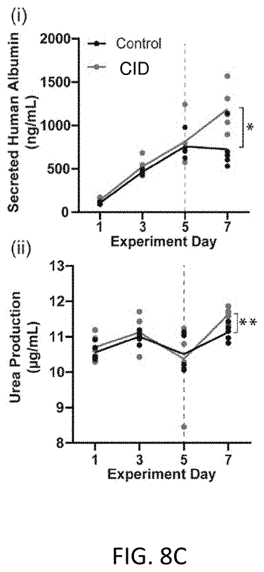

Given that fibroblasts are only transiently required for supporting the formation of a functional vasculature, it was investigated whether CAMEO could be used in the vascularization of tissue constructs with organ-specific parenchymal cells without negatively affecting the function of the parenchymal cells. To investigate this, a tri-culture system of HUVECs, iCasp9-HDFs, and primary human hepatocytes was employed in the microfluidic device. Using the methods described in Example 5, a solution of HUVECs (3 million cells/mL), iCasp-9 HDFs (total of 6 million cells/mL), hepatic aggregates (0.36 million aggregates/mL with about 150 hepatocytes per aggregate), fibrinogen (2.5 mg/mL), thrombin (1 U/mL) was made in a 1:1 mixed media of EGM-2 and hepatocyte maintenance media. After 5 days of tri-culture, the devices were treated with 50 nM CID to remove the iCasp9-HDFs, or with vehicle control. All samples were fixed at day 7 to evaluate vascular perfusion (FIG. 8A). In both groups, HUVECs formed perfusable vasculature permeating the hepatocyte-laden construct (FIG. 8B). To evaluate whether the hepatocytes remained functional after CID treatment, levels of albumin and urea, markers for hepatic protein synthesis function and nitrogen metabolism, respectively, were measured in the media. Removal of the iCasp-9 HDFs did not negatively affect the secretive functions of the hepatocytes in the devices (FIG. 8C). In fact, a statistically higher level of secreted albumin and urea was observed at day 7 in CAMEO devices versus the vehicle control, suggesting potential benefits of removing the fibroblast population in the engineered liver constructs.

### Example 9: CAMEO Enables In Vivo Transplantation of Tissue Constructs

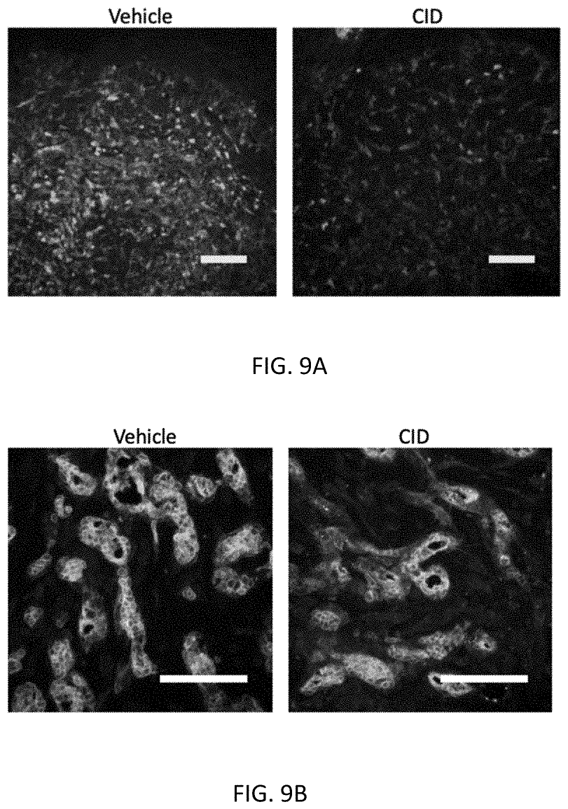

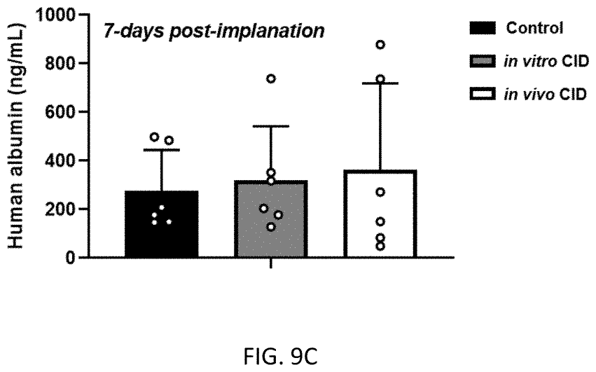

The ability to successfully transplant the described tissue constructs was assessed in mice. Specifically, engineered tissue constructs consisting of HUVECs and iCasp9-human lung fibroblasts (iCasp9-HLF, see Example 7) were cultured for 7 days in vitro. Constructs were then implanted into the intraperitoneal mesenteric parametrial fat pad of mice. Five days after implantation, mice were injected with 10 mg/kg CID or vehicle intraperitoneally. Mice were dosed again on days 7 and 9. Constructs were collected from mice on day 10 post-implantation. Explants were cryosectioned and imaged to visualize the presence of cells. CID treated animals showed a reduction in iCasp9-HLF compared to vehicle treated mice (FIG. 9A). Additionally, the implanted tissues demonstrated the ability to successfully integrate with host vessels. Specifically, immunofluorescence staining for red blood cells (anti-TER119) in vessel lumens suggests that implanted tissue constructs supported the maintenance of engineered vessel structures, which were perfusable and anastomosed to host vasculature. Both the vehicle control and fibroblast depleted (CID treated) tissue constructs demonstrated vessel integration (FIG. 9B).

Following the confirmation of vessel maintenance and anastomosis and ability to induce death of implanted fibroblasts in vivo, hepatocyte function was evaluated in implanted hepatocyte tissue constructs. Specifically, mice were implanted with hepatocyte/iCasp9-HDF/HUVEC tissue constructs after in vitro culture for 7 days. Constructs were implanted in mouse fat pads as described above, and treated with 10 mg/kg CID or vehicle on days 5 and 6 post-implantation. At day 7, blood was collected and centrifuged to isolate plasma. When analyzed via ELISA, in vivo CID treated constructs demonstrated similar human albumin secretion compared to vehicle control and in vitro CID treated tissue constructs (FIG. 9C). These experiments demonstrate that CAMEO enables in vivo tissue implantation with maintained hepatocyte function and maintenance of perfusable vascular structures.

1. An engineered tissue construct comprising one or more mammalian cell populations, wherein at least one cell population comprises a genetic construct comprising:
(i) a polynucleotide encoding a polypeptide of interest comprising an inducible element, wherein the polypeptide is activated upon interaction of the inducible element with a biological molecule or small molecule; or
(ii) a polynucleotide comprising an inducible promoter operably linked to a nucleotide sequence encoding a polypeptide or a nucleic acid molecule of interest, wherein expression of the polypeptide or nucleic acid molecule is controlled by the inducible promoter, wherein at least one cell population comprises parenchymal or non-parenchymal cells.

2. The engineered tissue construct of claim 1, wherein the polypeptide of interest of (i) or (ii) is a cell death-inducing polypeptide.

3.-9. (canceled)

10. The engineered tissue construct of claim 1, wherein the polypeptide of interest of (i) or (ii) induces cell proliferation in at least one cell population.

11.-12. (canceled)

13. The engineered tissue construct of claim 10, wherein the polypeptide of interest is selected from the group consisting of Wnt2, epidermal growth factor (EGF), hepatocyte growth factor (HGF), fibroblast growth factor (FGF), vascular endothelial growth factor (VEGF), interleukin 8 (IL-8), angiotensin 2 (Ang-2), R-spondin-3 precursor (RSPO3), GATA-binding protein 4 (GATA4), interleukin 6 (IL-6), delta-like 4 (DLL4), inhibitor of DNA binding 1 (ID-1), prostaglandin E synthase 2 (PGE2) and colony stimulating factor 1 (CSF1).

14. The engineered tissue construct of claim 1, wherein the nucleic acid molecule of interest is an inhibitory nucleic acid molecule.

15.-25. (canceled)

26. The engineered tissue construct of claim 1, wherein the tissue construct comprises at least one population of parenchymal cells.

27. (canceled)

28. The engineered tissue construct of claim 26, wherein the parenchymal cells are hepatocytes or hepatocyte precursor cells.

29. The engineered tissue construct of claim 1, wherein the tissue construct comprises at least one population of non-parenchymal cells.

30. The engineered tissue construct of claim 29, wherein the non-parenchymal cells are stromal cells.

31. (canceled)

32. The engineered tissue construct of claim 29, wherein the non-parenchymal cells are endothelial cells.

33. The engineered tissue construct of claim 29, wherein the tissue construct comprises at least two populations of non-parenchymal cells.

34. (canceled)

35. The engineered tissue construct of claim 1, wherein the tissue construct comprises at least one population of parenchymal cells and at least one population of non-parenchymal cells.

36. The engineered tissue construct of claim 35, wherein the parenchymal cells are hepatocytes or hepatocyte precursor cells, and the non-parenchymal cells are stromal cells.

37. The engineered tissue construct of claim 35, wherein the tissue construct comprises two populations of non-parenchymal cells.

38. The engineered tissue construct of claim 37, wherein the parenchymal cells are hepatocytes, and wherein the two populations of non-parenchymal cells is a population of stromal cells and a population of endothelial cells.

39.-51. (canceled)

52. A method for eliminating a population of cells within an engineered tissue construct, comprising:
(a) introducing a genetic construct into a first cell population, wherein the genetic construct comprises (i) a polynucleotide encoding a cell death-inducing polypeptide operably linked to an inducible element, wherein the polypeptide is activated upon interaction of the inducible element with a biological molecule or small molecule, or (ii) a polynucleotide comprising an inducible promoter operably linked to a nucleotide sequence encoding a cell death-inducing polypeptide or nucleic acid molecule, wherein expression of the polypeptide or nucleic acid molecule is controlled by the inducible promoter;
(b) co-culturing the cells of (a) with a second cell population on a substrate to form the engineered tissue construct; and
(c) contacting the tissue construct with (i) the biological molecule or small molecule to activate the cell death-inducing polypeptide, or (ii) the inducible promoter to express the cell death-inducing polypeptide or nucleic acid molecule, such that the first cell population is eliminated from the tissue construct.

53.-60. (canceled)

61. A method for inducing expansion of an engineered tissue construct, comprising:
(a) introducing a genetic construct into at least one population of cells, wherein the genetic construct comprises (i) a polynucleotide encoding a polypeptide of interest comprising an inducible element, wherein the polypeptide is activated upon interaction of the inducible element with a biological molecule or small molecule, or (ii) a polynucleotide comprising an inducible promoter operably linked to a nucleotide sequence encoding a polypeptide or a nucleic acid molecule of interest, wherein expression of the polypeptide or nucleic acid molecule is controlled by the inducible promoter;
(b) culturing the cell population of (a), with or without another cell population, onto a substrate to form a tissue construct; and
(c) contacting the tissue construct with (i) the biological molecule or small molecule, or (ii) a stimulus of the inducible promoter, such that expression of the polypeptide induces expansion of the engineered tissue construct.

62.-69. (canceled)

70. The method of claim 61, wherein the polypeptide of interest is selected from the group consisting of Wnt2, epidermal growth factor (EGF), hepatocyte growth factor (HGF), fibroblast growth factor (FGF), vascular endothelial growth factor (VEGF), interleukin 8 (IL-8), angiotensin 2 (Ang-2), r-spondin-3 precursor (RSPO3), hemagglutinin (HA), GATA-binding protein 4 (GATA4), interleukin 6 (IL-6), delta-like 4 (DLL4), inhibitor of DNA binding 1 (ID-1), prostaglandin E synthase 2 (PTGES2) and colony stimulating factor 1 (CSF1).

71.-75. (canceled)

76. A method for treating a metabolic disorder in a subject in need thereof, the method comprising implanting the engineered tissue construct of claim 1 into the subject.

77.-79. (canceled)

80. A method of treating chronic liver failure in a subject, comprising implanting the engineered tissue construct of claim 1 into the subject.

81.-84. (canceled)
